(19) United States
(12) Patent Application Publication
(10) Pub. No.: US 2022/0226501 A1
(43) Pub. Date: Jul. 21, 2022

(54) Insulin gene therapy

(71) Applicant: Universitat Autònoma de Barcelona, Cerdanyola del Vallès (ES)

(72) Inventors: Fàtima Bosch Tubert, Cerdanyola del Vallès (ES); Ivet Elias Puigdomenech, Cerdanyola del Vallès (ES); Albert Ribera Sánchez, Santa Eulàlia de Ronçana (ES); Ignasi Grass Costa, Sabadell (ES)

(73) Assignee: Universitat Autònoma de Barcelona, Cerdanyola del Vallès (ES)

(21) Appl. No.: 17/614,390

(22) PCT Filed: May 29, 2020
(86) PCT No.: PCT/EP2020/065018

§ 371 Date: Nov. 26, 2021

(30) Foreign Application Priority Data
May 31, 2019 (EP) 19382447.1

(51) Int. Cl. A61K 48/00; A61K 38/28; C12N 15/113; A61P 25/28; C07K 14/62; C12N 15/86

(52) U.S. Cl. CPC A61K 48/005; A61K 38/28; C12N 15/1136; A61P 25/28; C07K 14/62; C12N 2320/32; A61K 48/0075; C12N 2310/141; C12N 2750/14143; C12N 2750/14171; C12N 15/86

(57) ABSTRACT

Described herein is a gene construct comprising a nucleotide sequence encoding insulin, for use in the treatment and/or prevention of neuroinflammation, neurodegeneration and/or cognitive decline, or a disease or condition associated therewith.

### FIELD

Aspects herein pertain to the medical field, comprising insulin gene therapy for use in the treatment of neuroinflammation, neurodegeneration and/or cognitive decline in mammals, particularly in human beings.

### BACKGROUND

Alzheimer disease (AD), diabetes and obesity are worldwide growing epidemics leading to reduced life expectancy and poor quality of life (IDF Atlas 2015, www.idf.org; Mayeux, R. et al. 2012, Cold Spring Harb. Perspect. Med. 2012, 2:a006239). Recent data has shown that inflammation and insulin resistance in the central nervous system (CNS) is a shared hallmark feature not only of diabetes and obesity but also of AD and other neuropathological processes underlying cognitive aging and dementia (De Felice, F. G., 2013, J. Clin. Invest. 123:531-539; Kullmann, S. et al. 2016, Physiol. Rev 96:1169-1209; Guillemot-Legris, O. et al., 2017, Trends Neurosci. 40:237-253; Dutheil S. et al. 2016, Neuropsychopharmacology. 41:1874-1887).

Some reports have shown that administration of recombinant insulin using the intranasal route to reach the central nervous system (CNS) improves memory function both in cognitively impaired individuals and normal adults (Craft, S. et al., 2012, Arch. Neurol. 69:29-38; Reger, M. A. et al., 2006, Neurobiology of aging, 27:451-458). Long-term intranasal insulin infusion in a rat model of AD also ameliorates cognition, reduces tau hyperphosphorylation, attenuates microglial activation and promotes neurogenesis (Guo, Z. et al., 2017, Sci. Rep. 7:1-12).

However, the pharmacokinetics of nasal human insulin spray are poor, and after intranasal insulin administration there is a peak of insulin in the cerebrospinal fluid (CSF) that is rapidly reduced after 60 minutes (Born, J., et al., 2002, Nat. Neurosci. 5(6):514-516). Therefore, this approach needs multiple administrations and there are several local side effects of long-term exposure of the nasal mucosa to insulin (Schmid, V. et al., 2018, Diabetes Obes Metab. 20:1563-1577).

Given the importance that neuroinflammation and neurogenesis play in cognitive decline, new therapeutic approaches to mitigate inflammation in the CNS and stimulate neurogenesis which do not have all the drawbacks of existing treatments may be of compelling importance.

### SUMMARY

In a first aspect, there is provided a gene construct comprising a nucleotide sequence encoding insulin, for use in the treatment and/or prevention of neuroinflammation, neurodegeneration and/or cognitive decline, or a disease or condition associated therewith.

In a preferred embodiment, the nucleotide sequence encoding insulin is operably linked to a ubiquitous promoter.

In another preferred embodiment, the ubiquitous promoter is selected from the group consisting of a CAG promoter and a CMV promoter, preferably the ubiquitous promoter is a CAG promoter.

In another preferred embodiment, the gene construct comprises at least one target sequence of a microRNA expressed in a tissue where the expression of insulin is wanted to be prevented, preferably wherein the at least one target sequence of a microRNA is selected from those target sequences that bind to microRNAs expressed in heart and/or liver of the mammal.

In another preferred embodiment, the gene construct comprises at least one target sequence of a microRNA expressed in the liver and at least one target sequence of a microRNA expressed in the heart, preferably a target sequence of a microRNA expressed in the heart is selected from SEQ ID NO's: 8 and 16-20 and a target sequence of a microRNA expressed in the liver is selected from SEQ ID NO's: 7 and 9-15, more preferably the gene construct comprises a target sequence of microRNA-122a (SEQ ID NO: 7) and a target sequence of microRNA-1 (SEQ ID NO: 8).

In another preferred embodiment, the nucleotide sequence encoding insulin is selected from the group consisting of:
(a) a nucleotide sequence encoding a polypeptide comprising an amino acid sequence that has at least 60% sequence identity with the amino acid sequence of SEQ ID NO: 1, 2 or 3;
(b) a nucleotide sequence that has at least 60% sequence identity with the nucleotide sequence of SEQ ID NO: 4, 5 or 6; and
(c) a nucleotide sequence the sequence of which differs from the sequence of a nucleotide sequence of (b) due to the degeneracy of the genetic code.

In a second aspect, there is provided an expression vector comprising a gene construct according to the first aspect, for use in the treatment and/or prevention of neuroinflammation, neurodegeneration and/or cognitive decline, or a disease or condition associated therewith.

In a preferred embodiment, the expression vector is a viral vector, preferably wherein the expression vector is a viral vector selected from the group consisting of adenoviral vectors, adeno-associated viral vectors, retroviral vectors, and lentiviral vectors, more preferably an adeno-associated viral vector.

In a preferred embodiment, the expression vector is an adeno-associated viral vector of serotype 1, 2, 3, 4, 5, 6, 7, 8, 9, rh10, rh8, Cb4, rh74, DJ, 2/5, 2/1, 1/2 or Anc80, preferably an adeno-associated viral vector of serotype 1, 2 or 9, more preferably an adeno-associated viral vector of serotype 1 or 9.

In a third aspect, there is provided a pharmaceutical composition comprising a gene construct according to the first aspect and/or an expression vector according to the second aspect, together with one or more pharmaceutically acceptable ingredients, for use in the treatment and/or prevention of neuroinflammation, neurodegeneration and/or cognitive decline, or a disease or condition associated therewith.

Also provided is a gene construct for use according to the first aspect and/or an expression vector for use according to the second aspect and/or a pharmaceutical composition for use according to the third aspect, wherein the disease or condition associated with neuroinflammation, neurodegeneration and/or cognitive disorder is selected from the group consisting of: a cognitive disorder, dementia, Alzheimer's disease, vascular dementia, Lewy body dementia, frontotemporal dementia (FTD), Parkinson's disease, Parkinson-like disease, Parkinsonism, Huntington's disease, traumatic brain injury, prion disease, dementia/neurocognitive issues due to HIV infection, dementia/neurocognitive issues due to aging, tauopathy, multiple sclerosis and other neuroinflammatory/neurodegenerative diseases, preferably Alzheimer's disease, Parkinson's disease and/or Parkinson-like disease, more preferably Alzheimer's disease or Parkinson's disease.

In some embodiments, the gene construct and/or expression vector and/or pharmaceutical composition is administered by intra-CSF administration.

In another aspect, there is provided a gene construct comprising a nucleotide sequence encoding insulin wherein the nucleotide sequence encoding insulin is operably linked to a ubiquitous promoter and wherein the gene construct comprises at least one target sequence of a microRNA expressed in a tissue where the expression of insulin is wanted to be prevented, preferably wherein the at least one target sequence of a microRNA is selected from those target sequences that bind to microRNAs expressed in heart and/or liver of the mammal.

In a preferred embodiment, the gene construct comprises at least one target sequence of a microRNA expressed in the liver and at least one target sequence of a microRNA expressed in the heart, preferably a microRNA expressed in the heart is selected from SEQ ID NO's: 8 and 16-20 and a target sequence of a microRNA expressed in the liver is selected from SEQ ID NO's: 7 and 9-15, more preferably the gene construct comprises a target sequence of microRNA-122a (SEQ ID NO: 7) and a target sequence of microRNA-1 (SEQ ID NO: 8).

In another aspect, there is provided an expression vector comprising a gene construct as defined in the previous aspect, preferably wherein the expression vector is a viral vector, more preferably wherein the expression vector is a viral vector selected from the group consisting of adenoviral vectors, adeno-associated viral vectors, retroviral vectors, and lentiviral vectors, most preferably wherein the expression vector is an adeno-associated viral vector.

### DESCRIPTION

The present inventors have developed an improved gene therapy strategy based on insulin gene therapy directed to the central nervous system (CNS) to counteract neuroinflammation, neurodegeneration and/or cognitive decline. The long-term and effective expression of insulin provided by a single intra-CSF administration of the vectors of the present invention represents a significant advantage over other therapies. Particularly, as elaborated in the experimental part, the present inventors have found the following unexpected advantages of brain-directed insulin gene therapy:
The gene constructs and vectors as described herein can obtain a robust and wide-spread overexpression in the brain, including hypothalamus, cortex, hippocampus, cerebellum and olfactory bulb (Examples 1, 2, 3, 5).
In a widely used mouse model of senescence with age-related brain pathologies such as neuroinflammation, expression of insulin in the brain using gene constructs and vectors according to the invention led to a clear reduction in neuroinflammation, increased neurogenesis and increased astrocyte numbers (Example 1) as well as amelioration of short-term memory, long-term memory and learning capacity (Example 5).
In a widely used mouse model of obesity and diabetes, associated with neuroinflammation and cognitive decline, expression of insulin in the brain using gene constructs and vectors according to the invention led to a clear reduction in neuroinflammation and increased astrocyte numbers (Example 2).

Accordingly, the aspects and embodiments of the present invention as described herein solve at least some of the problems and needs as discussed herein.

### Gene Construct

In a first aspect, there is provided a gene construct comprising a nucleotide sequence encoding insulin. Preferably, gene constructs as described herein are for use as a medicament. More preferably, gene constructs as described herein are for use in the treatment and/or prevention of neuroinflammation, neurodegeneration and/or cognitive decline, or a disease or condition associated therewith.

A “gene construct” as described herein has its customary and ordinary meaning as understood by one of skill in the art in view of this disclosure. A “gene construct” can also be called an “expression cassette” or “expression construct” and refers to a gene or a group of genes, including a gene that encodes a protein of interest, which is operatively linked to a promoter that controls its expression. The part of this application entitled “general information” comprises more detail as to a “gene construct”. “Operatively linked” as used herein is further described in the part of this application entitled “general information”.

In some embodiments, a gene construct as described herein is suitable for expression in a mammal. As used herein, “suitable for expression in a mammal” may mean that the gene construct includes one or more regulatory sequences, selected on the basis of the mammalian host cells to be used for expression, operatively linked to the nucleotide sequence to be expressed. Preferably, said mammalian host cells to be used for expression are human, murine or canine cells.

In some embodiments, the gene construct as described herein comprises a nucleotide sequence encoding an insulin to be expressed in the CNS, preferably in the brain, optionally in the CNS and/or brain of a mammal. In some embodiments, the gene construct as described herein is suitable for expression in the CNS, preferably in the brain. In some embodiments, expression of the gene construct in the brain may mean expression of the gene construct in the hypothalamus and/or the cortex and/or the hippocampus and/or the cerebellum and/or the olfactory bulb. Accordingly, expression of the gene construct in the brain may mean expression of the gene construct in at least one or at least two or at least three or all brain regions selected from the group consisting of the hypothalamus, the cortex, the hippocampus, the cerebellum and the olfactory bulb. Expression may be assessed using techniques such as qPCR, Western blot analysis or ELISA as described under the section entitled “general information”.

In the context of embodiments of the invention, an insulin to be expressed in the CNS and/or the brain; and a gene construct suitable for expression in the CNS and/or the brain, refer to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least two-fold higher, at least three-fold higher, at least four-fold higher, at least five-fold higher, at least six-fold higher, at least seven-fold higher, at least eight-fold higher, at least nine-fold higher, at least ten-fold higher, or more) expression of insulin in the CNS and/or the brain as compared to other organs or tissues. Other organs or tissues may be the liver, pancreas, adipose tissue, skeletal muscle, heart, kidney, colon, hematopoietic tissue, lung, ovary, spleen, stomach, testis and others. Preferably, other organs are the liver and/or the heart. Other organs may also be skeletal muscle. In an embodiment, expression is not detectable in the liver, pancreas, adipose tissue, skeletal muscle, heart, kidney, colon, hematopoietic tissue, lung, ovary, spleen, stomach and/or testis. In a preferred embodiment, expression is not detectable in the liver and/or the heart. In another preferred embodiment, expression is not detectable in the skeletal muscle. In some embodiments, expression is not detectable in at least one, at least two, at least three, at least four or all organs selected from the group consisting of the liver, pancreas, adipose tissue, skeletal muscle, heart, kidney, colon, hematopoietic tissue, lung, ovary, spleen, stomach and testis. Expression may be assessed using techniques such as qPCR, Western blot analysis or ELISA as described under the section entitled “general information”.

In some embodiments, the nucleotide sequence encoding insulin is operably linked to a ubiquitous promoter.

In some embodiments, a ubiquitous promoter as described herein is selected from the group consisting of a CAG promoter, a CMV promoter, a mini-CMV promoter, a β-actin promoter, a rous-sarcoma-virus (RSV) promoter, an elongation factor 1 alpha (EF1α) promoter, an early growth response factor-1 (Egr-1) promoter, an Eukaryotic Initiation Factor 4A (eIF4A) promoter, a ferritin heavy chain-encoding gene (FerH) promoter, a ferritin heavy light-encoding gene (FerL) promoter, a glyceraldehyde-3-phosphate dehydrogenase (GAPDH) promoter, a GRP78 promoter, a GRP94 promoter, a heat-shock protein 70 (hsp70) promoter, an ubiquitin B promoter, a SV40 promoter, a Beta-Kinesin promoter, a ROSA26 promoter and a PGK-1 promoter.

In a preferred embodiment, a ubiquitous promoter may be selected from the group consisting of a CAG promoter and a CMV promoter. In a preferred embodiment, the ubiquitous promoter is a CAG promoter. CAG promoters are demonstrated in the examples to be suitable for use in a gene construct according to the invention.

In some embodiments, a CAG promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 22. In some embodiments, identity may be assessed relative to a part of SEQ ID NO: 22, such as at least 50%, 60%, 70%, 80%, 90%, 95% or 100% of SEQ ID NO: 22.

Another preferred ubiquitous promoter is a cytomegalovirus (CMV) promoter. In some embodiments, a CMV promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 23. In some embodiments, identity may be assessed relative to a part of SEQ ID NO: 23, such as at least 50%, 60%, 70%, 80%, 90%, 95% or 100% of SEQ ID NO: 23.

Preferably said CMV promoter is used together with an intronic sequence. In some embodiments, an intronic sequence comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 21. In some embodiments, identity may be assessed relative to a part of SEQ ID NO: 21, such as at least 50%, 60%, 70%, 80%, 90%, 95% or 100% of SEQ ID NO: 21.

Another preferred ubiquitous promoter is a mini-CMV promoter. In some embodiments, a mini-CMV promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 25. In some embodiments, identity may be assessed relative to a part of SEQ ID NO: 25, such as at least 50%, 60%, 70%, 80%, 90%, 95% or 100% of SEQ ID NO: 25.

Another preferred ubiquitous promoter is an EF1α promoter. In some embodiments, an EF1α promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 26. In some embodiments, identity may be assessed relative to a part of SEQ ID NO: 26, such as at least 50%, 60%, 70%, 80%, 90%, 95% or 100% of SEQ ID NO: 26.

Another preferred ubiquitous promoter is an RSV promoter. In some embodiments, an RSV promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 27. In some embodiments, identity may be assessed relative to a part of SEQ ID NO: 27, such as at least 50%, 60%, 70%, 80%, 90%, 95% or 100% of SEQ ID NO: 27.

In some embodiments, the gene construct comprises at least one target sequence of a microRNA expressed in a tissue where the expression of insulin is wanted to be prevented. In some embodiments, the nucleotide sequence encoding insulin is operably linked to a ubiquitous promoter and the gene construct comprises at least one target sequence of a microRNA expressed in a tissue where the expression of insulin is wanted to be prevented.

A description of “ubiquitous promoter”, “operably linked” and “microRNA” has been provided under the section entitled “general information”. A “target sequence of a microRNA expressed in a tissue” or “target sequence binding to a microRNA expressed in a tissue” or “binding site of a microRNA expressed in a tissue” as used herein refers to a nucleotide sequence which is complementary or partially complementary to at least a portion of a microRNA expressed in said tissue, as described elsewhere herein. Expression may be assessed using techniques such as qPCR, Western blot analysis or ELISA as described under the section entitled “general information”.

In some embodiments, the at least one target sequence of a microRNA is selected from those target sequences that bind to microRNAs expressed in heart and/or liver of a mammal. Preferably, in some embodiments, the gene construct comprises at least one target sequence of a microRNA expressed in the liver and at least one target sequence of a microRNA expressed in the heart.

A “target sequence of a microRNA expressed in the liver” or “target sequence binding to a microRNA expressed in the liver” or “binding site of a microRNA expressed in the liver” as used herein refers to a nucleotide sequence which is complementary or partially complementary to at least a portion of a microRNA expressed in the liver. Similarly, a “target sequence of a microRNA expressed in the heart” or “target sequence binding to a microRNA expressed in the heart” or “binding site of a microRNA expressed in the heart” as used herein refers to a nucleotide sequence which is complementary or partially complementary to at least a portion of a microRNA expressed in the heart.

A portion of a microRNA expressed in the liver or a portion of a microRNA expressed in the heart, as described herein, means a nucleotide sequence of at least four, at least five, at least six or at least seven consecutive nucleotides of said microRNA. The binding site sequence can have perfect complementarity to at least a portion of an expressed microRNA, meaning that the sequences are a perfect match without any mismatch occurring. Alternatively, the binding site sequence can be partially complementary to at least a portion of an expressed microRNA, meaning that one mismatch in four, five, six or seven consecutive nucleotides may occur. Partially complementary binding sites preferably contain perfect or near perfect complementarity to the seed region of the microRNA, meaning that no mismatch (perfect complementarity) or one mismatch per four, five, six or seven consecutive nucleotides (near perfect complementarity) may occur between the seed region of the microRNA and its binding site. The seed region of the microRNA consists of the 5′ region of the microRNA from about nucleotide 2 to about nucleotide 8 of the microRNA. The portion as described herein is preferably the seed region of said microRNA. Degradation of the messenger RNA (mRNA) containing the target sequence for a microRNA expressed in the liver or a microRNA expressed in the heart may be through the RNA interference pathway or via direct translational control (inhibition) of the mRNA. This invention is in no way limited by the pathway ultimately utilized by the miRNA in inhibiting expression of the transgene or encoded protein.

In the context of the invention, a target sequence that binds to microRNAs expressed in the liver may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 7 or 9-15. In some embodiments, a target sequence that binds to microRNAs expressed in the liver may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with a contiguous stretch of 4, 5, 6, 7, 8, 9, 10, 11, 12, 13, 14, 15, 16, 17, 18, 19, 20, 21, 22, 23 or more nucleotides of SEQ ID NO: 7 or 9-15.

In a preferred embodiment, the target sequence of a microRNA expressed in the liver may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 7. In some embodiments, a target sequence that binds to microRNAs expressed in the liver may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with a contiguous stretch of 4, 5, 6, 7, 8, 9, 10, 11, 12, 13, 14, 15, 16, 17, 18, 19, 20, 21, 22, 23 or more nucleotides of SEQ ID NO: 7. In a further embodiment, at least one copy of a target sequence of a microRNA expressed in the liver as described herein, is present in the gene construct of the invention. In a further embodiment, two, three, four, five, six, seven or eight copies of a target sequence of a microRNA expressed in the liver as described herein, are present in the gene construct of the invention. In a preferred embodiment, one, two, three, four, five, six, seven or eight copies of the sequence miRT-122a (SEQ ID NO: 7) are present in the gene construct of the invention. A preferred number of copies of a target sequence of a microRNA expressed in the liver as described herein is four.

A target sequence of a microRNA expressed in the liver as used herein exerts at least a detectable level of activity of a target sequence of a microRNA expressed in the liver as known to a person of skill in the art. An activity of a target sequence of a microRNA expressed in the liver is to bind to its cognate microRNA expressed in the liver and, when operatively linked to a transgene, to mediate detargeting of transgene expression in the liver. This activity may be assessed by measuring the levels of transgene expression in the liver on the level of the mRNA or the protein by standard assays known to a person of skill in the art, such as qPCR, Western blot analysis or ELISA.

In the context of the invention, a target sequence of a microRNA expressed in the heart may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 8 or 16-20. In some embodiments, a target sequence of a microRNA expressed in the heart may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with a contiguous stretch of 4, 5, 6, 7, 8, 9, 10, 11, 12, 13, 14, 15, 16, 17, 18, 19, 20, 21, 22, 23 or more nucleotides of SEQ ID NO: 8 or 16-20.

In a preferred embodiment, the target sequence of a microRNA expressed in the heart may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 8. In some embodiments, a target sequence of a microRNA expressed in the heart may be replaced by a nucleotide sequence comprising a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with a contiguous stretch of 4, 5, 6, 7, 8, 9, 10, 11, 12, 13, 14, 15, 16, 17, 18, 19, 20, 21, 22, 23 or more nucleotides of SEQ ID NO: 8.

In a further embodiment, at least one copy of a target sequence of a microRNA expressed in the heart as described herein, is present in the gene construct of the invention. In a further embodiment, two, three, four, five, six, seven or eight copies of a target sequence of a microRNA expressed in the heart as described herein, are present in the gene construct of the invention. In a preferred embodiment, one, two, three, four, five, six, seven or eight copies of a nucleotide sequence encoding miRT-1 (SEQ ID NO: 8), are present in the gene construct of the invention. A preferred number of copies of a target sequence of a microRNA expressed in the heart as described herein is four.

A target sequence of a microRNA expressed in the heart as used herein exerts at least a detectable level of activity of a target sequence of a microRNA expressed in the heart as known to a person of skill in the art. An activity of a target sequence of a microRNA expressed in the heart is to bind to its cognate microRNA expressed in the heart and, when operatively linked to a transgene, to mediate detargeting of transgene expression in the heart. This activity may be assessed by measuring the levels of transgene expression in the heart on the level of the mRNA or the protein by standard assays known to a person of skill in the art, such as qPCR, Western blot analysis or ELISA.

In some embodiments, at least one copy of a target sequence of a microRNA expressed in the liver as described herein, and at least one copy of a target sequence of a microRNA expressed in the heart as described herein, are present in the gene construct of the invention. In a further embodiment, two, three, four, five, six, seven or eight copies of a target sequence of a microRNA expressed in the liver as described herein, and two, three, four, five, six, seven or eight copies of a target sequence of a microRNA expressed in the heart as described herein, are present in the gene construct of the invention. In a further embodiment one, two, three, four, five, six, seven or eight copies of a nucleotide sequence encoding miRT-122a (SEQ ID NO: 7) and one, two, three, four, five, six, seven or eight copies nucleotide sequence encoding miRT-1 (SEQ ID NO: 8) are combined in the gene construct of the invention. In a further embodiment, four copies of a nucleotide sequence encoding miRT-122a (SEQ ID NO: 7) and four copies of nucleotide sequence encoding miRT-1 (SEQ ID NO: 8) are combined in the gene construct of the invention.

In some embodiments there is provided a gene construct as described above, wherein the target sequence of a microRNA expressed in the liver and the target sequence of a microRNA expressed in the heart is selected from a group consisting of sequences SEQ ID NO: 7 to 20 and/or combinations thereof. In some embodiments there is provided a gene construct as described above, wherein the target sequence of a microRNA expressed in the heart is selected from SEQ ID NO's: 8 and 16-20 and a target sequence of a microRNA expressed in the liver is selected from SEQ ID NO's: 7 and 9-15. In some embodiments there is provided a gene construct as described above, wherein the gene construct comprises a target sequence of microRNA-122a (SEQ ID NO: 7) and a target sequence of microRNA-1 (SEQ ID NO: 8).

A target sequence of a microRNA expressed in the liver and/or a target sequence of a microRNA expressed in the heart as described herein exerts at least a detectable level of an activity. An activity of a target sequence of a microRNA can be the degradation of the mRNA containing the target sequence of said microRNA. This degradation could be assessed using any technique known to the skilled person, for example by measuring expression/presence of said mRNA. Expression may be assessed using techniques such as qPCR, Western blot analysis or ELISA as described under the section entitled “general information”.

A nucleotide sequence encoding an insulin present in a gene construct according to the invention may be derived from any insulin gene or insulin coding sequence, including mutated insulin gene or insulin coding sequence, or codon optimized insulin gene or insulin coding sequence. In some embodiments, a nucleotide sequence encoding an insulin is a murine, canine, or human insulin gene or insulin coding sequence, a murine, canine, or human mutated insulin gene or insulin coding sequence, or a murine, canine, or human codon optimized insulin gene or insulin coding sequence. In some embodiments, a nucleotide sequence encoding an insulin is an insulin gene or insulin coding sequence from human, chimpanzee, mouse, rat or dog; or a mutated insulin gene or insulin coding sequence from human, chimpanzee, mouse, rat or dog; or a codon optimized insulin gene or insulin coding sequence from human, chimpanzee, mouse, rat or dog. A human sequence is preferred.

In a preferred embodiment, the nucleotide sequence encoding an insulin present in a gene construct according to the invention encodes an engineered insulin with furin cleavage sites. Such engineered insulin with furin cleavage sites is known to be processed in a highly efficient way to produce mature insulin in non-pancreatic tissues. In some embodiments, the nucleotide sequence encoding an engineered insulin with furin cleavage sites is selected from the group consisting of:
(a) a nucleotide sequence encoding a polypeptide comprising an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity or similarity with the amino acid sequence of SEQ ID NO: 41 or 42;
(b) a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with the nucleotide sequence of SEQ ID NO: 45 or 46; and
(c) a nucleotide sequence the sequence of which differs from the sequence of a nucleotide sequence of (a) or (b) due to the degeneracy of the genetic code.

Accordingly, in some embodiments, a preferred nucleotide sequence encoding an insulin encodes a polypeptide comprising an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with SEQ ID NO: 1-3 or 41-44. SEQ ID NO: 1 represents an amino acid sequence of human insulin. SEQ ID NO: 2 represents an amino acid sequence of murine insulin. SEQ ID NO: 3 represents an amino acid sequence of canine insulin. SEQ ID NO: 41 represents an amino acid sequence of human insulin with furin cleavage sites. SEQ ID NO: 42 represents an amino acid sequence of human insulin mutant His-B10-Asp with furin cleavage sites. SEQ ID NO: 43 represents an amino acid sequence of murine insulin. SEQ ID NO: 44 represents an amino acid sequence of chimpanzee insulin. In some embodiments, identity may be assessed relative to a part of SEQ ID NO: 1-3 or 41-44, such as at least 50%, 60%, 70%, 80%, 90%, 95% or 100% of SEQ ID NO: 1-3 or 41-44.

In some embodiments, a nucleotide sequence encoding an insulin present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with any sequence selected from the group consisting of SEQ ID NO's: 4-6 or 45-48. SEQ ID NO: 4 represents a nucleotide sequence of human insulin. SEQ ID NO: 5 represents a nucleotide sequence of murine insulin. SEQ ID NO: 6 represents a nucleotide sequence of canine insulin. SEQ ID NO: 45 represents a nucleotide sequence of human insulin with furin cleavage sites. SEQ ID NO: 46 represents a nucleotide sequence of human insulin mutant His-B10-Asp with furin cleavage sites. SEQ ID NO: 47 represents a nucleotide sequence of murine insulin. SEQ ID NO: 48 represents a nucleotide sequence of chimpanzee insulin. In some embodiments, identity may be assessed relative to a part of SEQ ID NO: 4-6 or 45-48, such as at least 50%, 60%, 70%, 80%, 90%, 95% or 100% of SEQ ID NO: 4-6 or 45-48.

A description of “identity” or “sequence identity” and “similarity” or “sequence similarity” has been provided under the section entitled “general information”.

In some embodiments, a nucleotide sequence encoding a human insulin present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 4. In some embodiments, identity may be assessed relative to a part of SEQ ID NO: 4, such as at least 50%, 60%, 70%, 80%, 90%, 95% or 100% of SEQ ID NO: 4.

In some embodiments, a nucleotide sequence encoding murine insulin present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 5 or 47. In some embodiments, identity may be assessed relative to a part of SEQ ID NO: 5 or 47, such as at least 50%, 60%, 70%, 80%, 90%, 95% or 100% of SEQ ID NO: 5 or 47.

In some embodiments, a nucleotide sequence encoding canine insulin present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 6. In some embodiments, identity may be assessed relative to a part of SEQ ID NO: 6, such as at least 50%, 60%, 70%, 80%, 90%, 95% or 100% of SEQ ID NO: 6.

In some embodiments, a nucleotide sequence encoding human insulin present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 45 or 46. In some embodiments, identity may be assessed relative to a part of SEQ ID NO: 45 or 46, such as at least 50%, 60%, 70%, 80%, 90%, 95% or 100% of SEQ ID NO: 45 or 46.

In some embodiments, a nucleotide sequence encoding chimpanzee insulin present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 48. In some embodiments, identity may be assessed relative to a part of SEQ ID NO: 48, such as at least 50%, 60%, 70%, 80%, 90%, 95% or 100% of SEQ ID NO: 48.

In some embodiments, there is provided a gene construct as described herein, wherein the nucleotide sequence encoding an insulin is selected from the group consisting of:
(a) a nucleotide sequence encoding a polypeptide comprising an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity or similarity with the amino acid sequence of SEQ ID NO: 1-3 or 41-44;
(b) a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with the nucleotide sequence of SEQ ID NO: 4-6 or 45-48; and
(c) a nucleotide sequence the sequence of which differs from the sequence of a nucleotide sequence of (a) or (b) due to the degeneracy of the genetic code.

An insulin encoded by the nucleotide sequences described herein (especially when the insulin sequence is described as having a minimal identity percentage with a given SEQ ID NO) exerts at least a detectable level of an activity of an insulin. An activity of an insulin can be the regulation of hyperglycemia. More appropriately, in the context of this disclosure, an activity of an insulin could be assessed at the level of the insulin signaling cascade. For example, the phosphorylation status of different proteins of the insulin signaling cascade can be determined, such as tyrosine phosphorylation of IRS-1/2, phosphorylation of AKT, etc. Phosphorylation status can be assessed for example by Western blot analysis using antibodies recognizing phosphorylated tyrosine residues and/or antibodies which specifically recognize the phosphorylated form of the protein such as IRS-1/2 and AKT. An activity of an insulin can also be to decrease neuroinflammation, increase neurogenesis, or increase astrocytes. This activity could be assessed by methods known to a person of skill in the art, for example by measuring expression levels of inflammatory molecules, astrocyte markers and/or neurogenic markers as described in the experimental section.

The table below summarizes the sequence identity on the DNA and protein level for a representative number of insulin sequences which are suitable to be used in the gene constructs of this invention.

TABLE 1
Identity % determined by pairwise alignment. HomoloGene was used
with default parameters (https://www.ncbi.nlm.nih.gov/homologene).

| | Identity (%) | |
|---|---|---|
| Species | Protein | DNA |
| H. sapiens | (SEQ ID NO: 1) | (SEQ ID NO: 4) |
| vs. P. troglodytes | 98.2 (SEQ ID NO: 44) | 98.2 (SEQ ID NO: 48) |
| vs. C. lupus | 88.2 (SEQ ID NO: 3) | 86.1 (SEQ ID NO: 6) |
| vs. M. musculus | 81.8 (SEQ ID NO: 2) | 82.4 (SEQ ID NO: 5) |
| vs. R. norvegicus | 82.7 (SEQ ID NO: 43) | 81.2 (SEQ ID NO: 47) |
| P. troglodytes | (SEQ ID NO: 44) | (SEQ ID NO: 48) |
| vs. H. sapiens | 98.2 (SEQ ID NO: 1) | 98.2 (SEQ ID NO: 4) |
| vs. C. lupus | 87.3 (SEQ ID NO: 3) | 85.8 (SEQ ID NO: 6) |
| vs. M. musculus | 80.9 (SEQ ID NO: 2) | 82.1 (SEQ ID NO: 5) |
| vs. R. norvegicus | 81.8 (SEQ ID NO: 43) | 81.2 (SEQ ID NO: 47) |
| C. lupus | (SEQ ID NO: 3) | (SEQ ID NO: 6) |
| vs. H. sapiens | 88.2 (SEQ ID NO: 1) | 86.1 (SEQ ID NO: 4) |
| vs. P. troglodytes | 87.3 (SEQ ID NO: 44) | 85.8 (SEQ ID NO: 48) |
| vs. M. musculus | 80.9 (SEQ ID NO: 2) | 81.8 (SEQ ID NO: 5) |
| vs. R. norvegicus | 80.0 (SEQ ID NO: 43) | 80.0 (SEQ ID NO: 47) |
| M. musculus | (SEQ ID NO: 2) | (SEQ ID NO: 5) |
| vs. H. sapiens | 81.8 (SEQ ID NO: 1) | 82.4 (SEQ ID NO: 4) |
| vs. P. troglodytes | 80.9 (SEQ ID NO: 44) | 82.1 (SEQ ID NO: 48) |
| vs. C. lupus | 80.9 (SEQ ID NO: 3) | 81.8 (SEQ ID NO: 6) |
| vs. R. norvegicus | 94.5 (SEQ ID NO: 43) | 94.2 (SEQ ID NO: 47) |
| R. norvegicus | (SEQ ID NO: 43) | (SEQ ID NO: 47) |
| vs. H. sapiens | 82.7 (SEQ ID NO: 1) | 81.2 (SEQ ID NO: 4) |
| vs. P. troglodytes | 81.8 (SEQ ID NO: 44) | 81.2 (SEQ ID NO: 48) |
| vs. C. lupus | 80.0 (SEQ ID NO: 3) | 80.0 (SEQ ID NO: 6) |
| vs. M. musculus | 94.5 (SEQ ID NO: 2) | 94.2 (SEQ ID NO: 5) |
| H. sapiens | (SEQ ID NO: 1) | (SEQ ID NO: 4) |
| vs. H. Sapiens furin | 97.2 (SEQ ID NO: 41) | 97.8 (SEQ ID NO: 45) |
| vs. H. Sapiens furins asp | 96.3 (SEQ ID NO: 42) | 97.2 (SEQ ID NO: 46) |

In some embodiments, the nucleotide sequence encoding insulin is operably linked to a tissue-specific promoter. In a preferred embodiment, a tissue-specific promoter is a CNS-specific promoter, more preferably a brain-specific promoter. A CNS- and/or brain-specific promoter, as used herein, also encompasses promoters directing expression in a specific region or cellular subset of the CNS and/or brain. Accordingly, CNS- and/or brain specific promoters may also be selected from a hippocampus-specific promoter, a cerebellum-specific promoter, a cortex-specific promoter, a hypothalamus-specific promoter and/or an olfactory bulb-specific promoter, or any combination thereof.

A description of “tissue-specific promoter” has been provided under the section entitled “general information”.

In some embodiments, a CNS-specific promoter as described herein is selected from the group consisting of a Synapsin 1 promoter, a Neuron-specific enolase (NSE) promoter, a Calcium/calmodulin-dependent protein kinase II (CaMKII) promoter, a tyrosine hydroxylase (TH) promoter, a Forkhead Box A2 (FOXA2) promoter, an alpha-internexin (INA) promoter, a Nestin (NES) promoter, a Glial fibrillary acidic protein (GFAP) promoter, an Aldehyde Dehydrogenase 1 Family Member L1 (ALDH1L1) promoter, a myelin-associated oligodendrocyte basic protein (MOBP) promoter, a Homeobox Protein 9 (HB9) promoter, a Gonadotropin-releasing hormone (GnRH) promoter and a Myelin basic protein (MBP) promoter.

In some embodiments, a brain-specific promoter as described herein is selected from the group consisting of a Synapsin 1 promoter, a Neuron-specific enolase (NSE) promoter, a Calcium/calmodulin-dependent protein kinase II (CaMKII) promoter, a tyrosine hydroxylase (TH) promoter, a Forkhead Box A2 (FOXA2) promoter, an alpha-internexin (INA) promoter, a Nestin (NES) promoter, a Glial fibrillary acidic protein (GFAP) promoter, an Aldehyde Dehydrogenase 1 Family Member L1 (ALDH1L1) promoter, a myelin-associated oligodendrocyte basic protein (MOBP) promoter, a Gonadotropin-releasing hormone (GnRH) promoter and a Myelin basic protein (MBP) promoter.

In a preferred embodiment, the CNS- and/or brain-specific promoter is a synapsin 1 promoter. In some embodiments, a synapsin 1 promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 28. In some embodiments, identity may be assessed relative to a part of SEQ ID NO: 28, such as at least 50%, 60%, 70%, 80%, 90%, 95% or 100% of SEQ ID NO: 28.

Another preferred CNS- and/or brain-specific promoter is a calcium/calmodulin-dependent protein kinase II (CaMKII) promoter. In some embodiments, a calcium/calmodulin-dependent protein kinase II (CaMKII) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 29. In some embodiments, identity may be assessed relative to a part of SEQ ID NO: 29, such as at least 50%, 60%, 70%, 80%, 90%, 95% or 100% of SEQ ID NO: 29.

Another preferred CNS- and/or brain-specific promoter is a Glial fibrillary acidic protein (GFAP) promoter. In some embodiments, a Glial fibrillary acidic protein (GFAP) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 30. In some embodiments, identity may be assessed relative to a part of SEQ ID NO: 30, such as at least 50%, 60%, 70%, 80%, 90%, 95% or 100% of SEQ ID NO: 30.

Another preferred CNS- and/or brain-specific promoter is a Nestin promoter. In some embodiments, a Nestin promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 31. In some embodiments, identity may be assessed relative to a part of SEQ ID NO: 31, such as at least 50%, 60%, 70%, 80%, 90%, 95% or 100% of SEQ ID NO: 31.

Another preferred CNS-specific promoter is a Homeobox Protein 9 (HB9) promoter. In some embodiments, a Homeobox Protein 9 (HB9) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 32. In some embodiments, identity may be assessed relative to a part of SEQ ID NO: 32, such as at least 50%, 60%, 70%, 80%, 90%, 95% or 100% of SEQ ID NO: 32.

Another preferred CNS- and/or brain-specific promoter is a tyrosine hydroxylase (TH) promoter. In some embodiments, a tyrosine hydroxylase (TH) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 33. In some embodiments, identity may be assessed relative to a part of SEQ ID NO: 33, such as at least 50%, 60%, 70%, 80%, 90%, 95% or 100% of SEQ ID NO: 33.

Another preferred CNS- and/or brain-specific promoter is a Myelin basic protein (MBP) promoter. In some embodiments, a Myelin basic protein (MBP) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 34. In some embodiments, identity may be assessed relative to a part of SEQ ID NO: 34, such as at least 50%, 60%, 70%, 80%, 90%, 95% or 100% of SEQ ID NO: 34.

In some embodiments, CNS- and/or brain-specific promoters as described herein direct expression of said nucleotide sequence in at least one cell of the CNS and/or brain. Preferably, said promoter directs expression in at least 10%, 20%, 30%, 40%, 40%, 60%, 70%, 80%, 90%, or 100% of cells of the CNS and/or the brain. A CNS- and/or brain-specific promoter, as used herein, also encompasses promoters directing expression in a specific region or cellular subset of the CNS and/or brain. Accordingly, CNS- and/or brain specific promoters as described herein may also direct expression in at least 10%, 20%, 30%, 40%, 40%, 60%, 70%, 80%, 90%, or 100% of cells of the hippocampus, the cerebellum, the cortex, the hypothalamus and/or the olfactory bulb. Expression may be assessed using techniques such as qPCR, Western blot analysis or ELISA as described under the section entitled “general information”.

A promoter as used herein (especially when the promoter sequence is described as having a minimal identity percentage with a given SEQ ID NO) should exert at least an activity of a promoter as known to a person of skill in the art. Preferably a promoter described as having a minimal identity percentage with a given SEQ ID NO should control transcription of the nucleotide sequence to which it is operably linked (i.e. at least a nucleotide sequence encoding an insulin) as assessed in an assay known to a person of skill in the art. For example, such assay could involve measuring expression of the transgene. Expression may be assessed using techniques such as qPCR, Western blot analysis or ELISA as described under the section entitled “general information”.

Additional sequences may be present in the gene construct of the invention. Exemplary additional sequences suitable herein include inverted terminal repeats (ITRs), an SV40 polyadenylation signal (SEQ ID NO: 37), a rabbit β-globin polyadenylation signal (SEQ ID NO: 38), a CMV enhancer sequence (SEQ ID NO: 24). Within the context of the invention, “ITRs” is intended to encompass one 5′ITR and one 3′ITR, each being derived from the genome of an AAV. Preferred ITRs are from AAV2 and are represented by SEQ ID NO: 35 (5′ ITR) and SEQ ID NO: 36 (3′ ITR). Within the context of the invention, it is encompassed to use the CMV enhancer sequence (SEQ ID NO: 24) and the CMV promoter sequence (SEQ ID NO: 23) as two separate sequences or as a single sequence (SEQ ID NO: 39). Each of these additional sequences may be present in a gene construct according to the invention. In some embodiments, there is provided a gene construct comprising a nucleotide sequence encoding insulin as described herein, further comprising one 5′ITR and one 3′ITR, preferably AAV2 ITRs, more preferably the AAV2 ITRs represented by SEQ ID NO: 30 (5′ ITR) and SEQ ID NO: 31 (3′ ITR). In some embodiments, there is provided a gene construct comprising a nucleotide sequence encoding insulin as described herein, further comprising a polyadenylation signal, preferably an SV40 polyadenylation signal (preferably represented by SEQ ID NO: 32) and/or a rabbit β-globin polyadenylation signal (preferably represented by SEQ ID NO: 33).

Optionally, additional nucleotide sequences may be operably linked to the nucleotide sequence(s) encoding an insulin, such as nucleotide sequences encoding signal sequences, nuclear localization signals, expression enhancers, and the like.

In some embodiments, there is provided a gene construct comprising a nucleotide sequence encoding insulin, optionally wherein the gene construct does not comprise a target sequence of a microRNA expressed in a tissue where the expression of insulin is wanted to be prevented.

In some embodiments, the level of sequence identity or similarity as used herein is preferably 70%. Another preferred level of sequence identity or similarity is 80%. Another preferred level of sequence identity or similarity is 90%. Another preferred level of sequence identity or similarity is 95%. Another preferred level of sequence identity or similarity is 99%.

### Expression Vector

Gene constructs described herein can be placed in expression vectors. Thus, in another aspect there is provided an expression vector comprising a gene construct as described herein. Preferably, expression vectors as described herein are for use as a medicament. Preferably, expression vectors as described herein are for use in the treatment and/or prevention of neuroinflammation, neurodegeneration and/or cognitive decline, or a disease or condition associated therewith.

A description of “expression vector” has been provided under the section entitled “general information”.

In some embodiments, the expression vector is a viral expression vector. In some embodiments, a viral vector may be a viral vector selected from the group consisting of adenoviral vectors, adeno-associated viral vectors, retroviral vectors and lentiviral vectors. A preferred viral vector is an adeno-associated viral vector.

A description of “viral expression vector” has been provided under the section entitled “general information”. An adenoviral vector is also known as an adenovirus derived vector, an adeno-associated viral vector is also known as an adeno-associated virus derived vector, a retroviral vector is also known as a retrovirus derived vector and a lentiviral vector is also known as a lentivirus derived vector. A preferred viral vector is an adeno-associated viral vector. A description of “adeno-associated viral vector” has been provided under the section entitled “general information”.

In some embodiments, the vector is an adeno-associated vector or adeno-associated viral vector or an adeno-associated virus derived vector (AAV) selected from the group consisting of AAV of serotype 1 (AAV1), AAV of serotype 2 (AAV2), AAV of serotype 3 (AAV3), AAV of serotype 4 (AAV4), AAV of serotype 5 (AAV5), AAV of serotype 6 (AAV6), AAV of serotype 7 (AAV7), AAV of serotype 8 (AAV8), AAV of serotype 9 (AAV9), AAV of serotype rh10 (AAVrh10), AAV of serotype rh8 (AAVrh8), AAV of serotype Cb4 (AAVCb4), AAV of serotype rh74 (AAVrh74), AAV of serotype DJ (AAVDJ), AAV of serotype 2/5 (AAV2/5), AAV of serotype 2/1 (AAV2/1), AAV of serotype 1/2 (AAV1/2), AAV of serotype Anc80 (AAVAnc80). In a preferred embodiment, the vector is an AAV of serotype 1, 2 or 9 (AAV1, AAV2, or AAV9). These AAV serotypes are demonstrated in the examples to be suitable for use as an expression vector according to the invention. In a particularly preferred embodiment, the expression vector is an adeno-associated viral vector of serotype 9 or 1.

In a preferred embodiment, the expression vector is an AAV1 or AAV9, preferably an AAV9, and comprises a gene construct comprising a nucleotide sequence encoding insulin wherein the gene construct comprises at least one target sequence of a microRNA expressed in a tissue where the expression of insulin is wanted to be prevented.

In another preferred embodiment, the expression vector is an AAV1 or AAV9, preferably an AAV1, and comprises a gene construct comprising a nucleotide sequence encoding insulin, optionally wherein the gene construct does not comprise a target sequence of a microRNA expressed in a tissue where the expression of insulin is wanted to be prevented.

In a preferred embodiment, the expression vector is AAV9-CAG-hlns-dmiRT, comprising a gene construct encoding human insulin operatively linked to a CAG promoter and miRNA target sequences miRT-1 and miRT-122a. Optionally, the gene construct further includes a rabbit β-globin polyadenylation signal. In another preferred embodiment, the expression vector is AAV1-CAG-hlns, comprising a gene construct encoding human insulin operatively linked to a CAG promoter. Optionally, the gene construct further includes a rabbit β-globin polyadenylation signal.

### Composition

In a further aspect there is provided a composition comprising a gene construct as described herein and/or a viral vector as described herein, optionally together with one or more pharmaceutically acceptable ingredients. Preferably, compositions as described herein are for use as a medicament. Preferably, compositions as described herein are for use in the treatment and/or prevention of neuroinflammation, neurodegeneration and/or cognitive decline, or a disease or condition associated therewith. Preferably, in some embodiments, the composition is a pharmaceutical composition. Such compositions as described herein may also be called gene therapy compositions.

As used herein, “pharmaceutically acceptable ingredients” may include pharmaceutically acceptable carriers, fillers, preservatives, solubilizers, vehicles, diluents and/or excipients. Accordingly, the one or more pharmaceutically acceptable ingredients may be selected from the group consisting of pharmaceutically acceptable carriers, fillers, preservatives, solubilizers, vehicles, diluents and/or excipients. Such pharmaceutically acceptable carriers, fillers, preservatives, solubilizers, vehicles, diluents and/or excipients may for instance be found in Remington: The Science and Practice of Pharmacy, 22nd edition. Pharmaceutical Press (2013).

A further compound may be present in a composition of the invention. Said compound may help in delivery of the composition. Suitable compounds in this context are: compounds capable of forming complexes, nanoparticles, micelles and/or liposomes that deliver each constituent as described herein, complexed or trapped in a vesicle or liposome through a cell membrane. Many of these compounds are known in the art. Suitable compounds comprise polyethylenimine (PEI), or similar cationic polymers, including polypropyleneimine or polyethylenimine copolymers (PECs) and derivatives; synthetic amphiphiles (SAINT-18); lipofectin™; DOTAP. A person of skill in the art will know which type of formulation is the most appropriate for a composition as described herein.

### Method and Use

In a further aspect, there is provided a gene construct as described herein, for use as a medicament. Further provided is an expression vector as described herein, for use as a medicament. Further provided is a pharmaceutical composition as described herein, for use as a medicament. Also provided is a gene construct as described herein, for use in the treatment and/or prevention of neuroinflammation, neurodegeneration and/or cognitive decline, or a disease or condition associated therewith. Further provided is an expression vector as described herein, for use in the treatment and/or prevention of neuroinflammation, neurodegeneration and/or cognitive decline, or a disease or condition associated therewith. Further provided is a pharmaceutical composition as described herein, for use in the treatment and/or prevention of neuroinflammation, neurodegeneration and/or cognitive decline, or a disease or condition associated therewith.

Accordingly, in some embodiments, a gene construct as described herein and/or an expression vector as described herein and/or a pharmaceutical composition as described herein is for use in the treatment and/or prevention of neuroinflammation. In some embodiments, a gene construct as described herein and/or an expression vector as described herein and/or a pharmaceutical composition as described herein is for use in the treatment and/or prevention of neurodegeneration. In some embodiments, a gene construct as described herein and/or an expression vector as described herein and/or a pharmaceutical composition as described herein is for use in the treatment and/or prevention of cognitive decline. In the context of the invention, “neuroinflammation”, “neurodegeneration” and “cognitive decline” may be replaced with “neuroinflammation or a disease or condition associated therewith”, “neurodegeneration or a disease or condition associated therewith” and “cognitive decline or a disease or condition associated therewith”, respectively.

In some embodiments, a disease or condition associated with neuroinflammation, neurodegeneration and/or cognitive decline may be a cognitive disorder, dementia, Alzheimer's disease, vascular dementia, Lewy body dementia, frontotemporal dementia (FTD), Parkinson's disease, Parkinson-like disease, Parkinsonism, Huntington's disease, traumatic brain injury, prion disease, dementia/neurocognitive issues due to HIV infection, dementia/neurocognitive issues due to aging, tauopathy, multiple sclerosis and other neuroinflammatory/neurodegenerative diseases. In a preferred embodiment, a disease or condition associated with neuroinflammation, neurodegeneration and/or cognitive decline may be Alzheimer's disease, Parkinson's disease and/or Parkinson-like disease, preferably Alzheimer's disease and/or Parkinson's disease.

Accordingly, a gene construct as described herein and/or an expression vector as described herein and/or a pharmaceutical composition as described herein may be seen as an anti-neuroinflammatory medicine, anti-neurodegeneration medicine, and/or an anti-cognitive decline medicine. Accordingly, it may also be seen as an anti-aging medicine. Embodiments disclosed herein may also be used to treat and/or prevent neuroinflammation, neurodegeneration and/or cognitive decline associated with any of the afore-mentioned conditions.

In some embodiments, a gene construct for use and/or an expression vector for use and/or a pharmaceutical composition for use as described herein involves expression of the gene construct in the CNS, preferably in the brain.

Preferably, according to some embodiments, a gene construct for use and/or an expression vector for use and/or a pharmaceutical composition for use as described herein is administered by intra-CSF administration.

In a further aspect there is provided a method of treatment, comprising administering a gene construct, an expression vector or a pharmaceutical composition as described herein. Preferably, the treatment method is for the treatment and/or prevention of neuroinflammation, neurodegeneration and/or cognitive decline, or a disease or condition associated therewith. In some embodiments, administering a gene construct, an expression vector or a pharmaceutical composition means administering to a subject in need thereof a therapeutically effective amount of a gene construct, an expression vector or a pharmaceutical composition.

In a further aspect there is provided a use of a gene construct, an expression vector or a pharmaceutical composition as described herein, for the manufacture of a medicament. Preferably, in some embodiments, said medicament is for use in the treatment and/or prevention of neuroinflammation, neurodegeneration and/or cognitive decline, or a disease or condition associated therewith.

In a further aspect there is provided a use of a gene construct, an expression vector or a pharmaceutical composition as described herein, for medical treatment. Preferably, in some embodiments, said medical treatment is the treatment and/or prevention of neuroinflammation, neurodegeneration and/or cognitive decline, or a disease or condition associated therewith.

In another aspect there is provided a method for improving memory and/or learning in a subject, the method comprising administering to the subject a gene construct as described herein and/or an expression vector as described herein and/or a composition as described herein. In a preferred embodiment, an effective amount of a gene construct, an expression vector or a composition is administered. As used herein, an “effective amount” is an amount sufficient to exert beneficial or desired results. In a preferred embodiment, the subject to be treated is an elderly subject and/or a subject diagnosed with a metabolic disorder or disease, preferably obesity and/or diabetes. In some embodiments, memory may be recognition and/or recall memory, preferably recognition memory. In some embodiments, memory may be sensory memory; short-term and/or long-term memory, preferably short-term memory and/or long-term memory. In some embodiments, memory may be implicit (or procedural) and/or explicit (or declarative) memory. In a preferred embodiment, memory may also by spatial memory. In some embodiments, learning may be spatial learning. Further description of the different types of memory are included in the section entitled “General information”.

In preferred embodiments according to a gene construct for use, an expression vector for use, a composition for use, a method and a use according to the invention, the subject to be treated is an elderly subject and/or a subject diagnosed with a metabolic disorder or disease. In other words, in some embodiments according to a gene construct for use, an expression vector for use, a composition for use, a method and a use according to the invention, neuroinflammation, neurodegeneration and/or cognitive decline, or a disease or condition associated therewith, is associated with and/or caused by aging and/or a metabolic disorder or disease. Complications of a metabolic disorder or disease may also be encompassed.

As used herein, an elderly subject may preferably mean a subject with age 50 years or older, preferably 55 years or older, more preferably 60 years or older and most preferably 65 years or older.

In other embodiments according to a gene construct for use, an expression vector for use, a composition for use, a method and a use according to the invention, the subject to be treated is not an elderly subject and/or is a subject with age 50 years or younger, 45 years or younger, 40 years or younger, 35 years or younger, 30 years or younger, 25 years or younger.

In other embodiments according to a gene construct for use, an expression vector for use, a composition for use, a method and a use according to the invention, the subject to be treated is a subject not diagnosed with a metabolic disorder or disease. In other words, in some embodiments according to a gene construct for use, an expression vector for use, a composition for use, a method and a use according to the invention, the central nervous system disorder or disease, or a condition associated therewith, is not associated with and/or caused by aging and/or a metabolic disorder or disease.

Metabolic disorders and diseases may include metabolic syndrome, diabetes, obesity, obesity-related comorbidities, diabetes-related comorbidities, hyperglycaemia, insulin resistance, glucose intolerance, hepatic steatosis, alcoholic liver diseases (ALD), non-alcoholic fatty liver disease (NAFLD), non-alcoholic steatohepatitis (NASH), coronary heart disease (CHD), hyperlipidemia, atherosclerosis, endocrinopathies, osteosarcopenic obesity syndrome (OSO), diabetic nephropathy, chronic kidney disease (CKD), cardiac hypertrophy, diabetic retinopathy, diabetic nephropathy, diabetic neuropathy, arthritis, sepsis, ocular neovascularization, neurodegeneration, dementia, and may also include depression, adenoma, carcinoma. Diabetes may include prediabetes, hyperglycaemia, Type 1 diabetes, Type 2 diabetes, maturity-onset diabetes of the young (MODY), monogenic diabetes, neonatal diabetes, gestational diabetes, brittle diabetes, idiopathic diabetes, drug- or chemical-induced diabetes, Stiff-man syndrome, lipoatrophic diabetes, latent autoimmune diabetes in adults (LADA). Obesity may include overweight, central/upper body obesity, peripheral/lower body obesity, morbid obesity, osteosarcopenic obesity syndrome (OSO), pediatric obesity, Mendelian (monogenic) syndromic obesity, Mendelian non-syndromic obesity, polygenic obesity. Preferred metabolic disorders or diseases are obesity and/or a diabetes.

In some embodiments according to a gene construct for use, an expression vector for use, a composition for use, a method and a use according to the invention, the subject to be treated is a subject at risk of developing neuroinflammation, neurodegeneration and/or cognitive decline, or a disease or condition associated therewith.

Within the context of gene constructs for use, expression vectors for use, pharmaceutical compositions for use, methods and uses according to the invention, the therapy and/or treatment and/or medicament may involve expression of the gene construct in the CNS, preferably the brain. In some embodiments, there is no detectable expression in other tissues than the CNS and/or the brain. In some embodiments, expression of the gene construct in the brain may mean expression of the gene construct in the hypothalamus and/or the cortex and/or the hippocampus and/or the cerebellum and/or the olfactory bulb. Accordingly, expression of the gene construct in the brain may mean expression of the gene construct in at least one or at least two or at least three or all brain regions selected from the group consisting of the hypothalamus, the cortex, the hippocampus, the cerebellum and the olfactory bulb. In some embodiments, expression in the CNS and/or the brain may mean specific expression in the CNS and/or the brain. In an embodiment, expression is not detectable in the liver, pancreas, adipose tissue, skeletal muscle, heart, kidney, colon, hematopoietic tissue, lung, ovary, spleen, stomach and/or testis. In a preferred embodiment, expression is not detectable in the liver and/or the heart. In another preferred embodiment, expression is not detectable in the skeletal muscle. In some embodiments, expression does not involve expression in at least one, at least two, at least three, at least four or all organs selected from the group consisting of the liver, pancreas, adipose tissue, skeletal muscle, heart, kidney, colon, hematopoietic tissue, lung, ovary, spleen, stomach, testis. A description of CNS- and/or brain-specific expression has been provided under the section entitled “general information”.

Expression may be assessed using techniques such as qPCR, Western blot analysis or ELISA as described under the section entitled “general information”. A description of “CNS”, “brain”, “hypothalamus”, “hippocampus”, “cerebellum”, “cortex” and “olfactory bulb” has been provided under the section entitled “general information”.

Within the context of gene constructs for use, expression vectors for use, pharmaceutical compositions for use, methods and uses according to the invention, a gene construct and/or an expression vector and/or a pharmaceutical composition and/or a medicament may be administered by intra-CSF (cerebrospinal fluid) administration (via cisterna magna, intrathecal or intraventricular delivery). A preferred mode of administration, optionally a preferred mode of administration in humans, is intraventricular.

Within the context of gene constructs for use, expression vectors for use, pharmaceutical compositions for use, methods and uses according to the invention, a gene construct and/or an expression vector and/or a pharmaceutical composition and/or a medicament may be administered by intraparenchymal administration.

Within the context of gene constructs for use, expression vectors for use, pharmaceutical compositions for use, methods and uses according to the invention, a gene construct and/or an expression vector and/or a pharmaceutical composition and/or a medicament may be administered by intranasal administration.

“Intra-CSF administration”, “intranasal administration”, “intraparenchymal administration” “intra-cisterna magna administration”, “intrathecal administration” and “intraventricular administration”, as used herein, are described in the part of this application entitled “general information”.

In a preferred embodiment, a treatment or a therapy or a use or the administration of a medicament as described herein does not have to be repeated. In some embodiments, a treatment or a therapy or a use or the administration of a medicament as described herein may be repeated each year or each 2, 3, 4, 5, 6, 7, 8, 9 or 10, including intervals between any two of the listed values, years.

The subject treated may be a higher mammal, such as a cat, a rodent, (preferably mice, rats, gerbils and guinea pigs, and more preferably mice and rats), a dog, or a human being.

Within the context of gene constructs for use, expression vectors for use, pharmaceutical compositions for use, methods and uses according to the invention, a gene construct and/or an expression vector and/or a pharmaceutical composition and/or a medicament as described herein preferably exhibits at least one, at least two, at least three, at least four, or all of the following:
decreasing neuroinflammation;
increasing neurogenesis;
increasing the number of astrocytes;
decreasing neurodegeneration;
alleviating a symptom (as described later herein); and
improving a parameter (as described later herein).

Decreasing neuroinflammation may mean that inflammation of nervous tissue is decreased. This could be assessed using techniques known to a person of skill in the art such as the measurement of (neuro)inflammatory markers, for example as done in the experimental part. Exemplary markers that could be used in this regard are II-1b, 11-6 and NfkB. In this context, “decrease” (respectively “improvement”) means at least a detectable decrease (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The decrease may be seen after at least one week, one month, six months, one year or more of treatment using a gene construct and/or an expression vector and/or a composition of the invention. Preferably, the decrease is observed after a single administration. In some embodiments, the decrease is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Increasing neurogenesis may mean that neurons are produced by neural stem cells. This could be assessed using techniques known to a person of skill in the art such as the measurement of neurogenesis markers, for example as done in the experimental part. Exemplary markers that could be used in this regard are Dcx, Ncam and Sox2. In this context, “increase” (respectively “improvement”) means at least a detectable increase (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The increase may be seen after at least one week, one month, six months, one year or more of treatment using a gene construct and/or an expression vector and/or a composition of the invention. Preferably, the increase is observed after a single administration. In some embodiments, the increase is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Increasing the number of astrocytes may mean that the number of astrocytes is increased. This could be assessed using techniques known to a person of skill in the art such as the measurement of astrocyte markers, for example as done in the experimental part. Exemplary markers that could be used in this regard are Gfap and S100b. In this context, “increase” (respectively “improvement”) means at least a detectable increase (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The increase may be seen after at least one week, one month, six months, one year or more of treatment using a gene construct and/or an expression vector and/or a composition of the invention. Preferably, the increase is observed after a single administration. In some embodiments, the increase is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Decreasing neurodegeneration may mean that the loss of structure or function of neurons, including death of neurons, is decreased. This could be assessed using techniques known to a person of skill in the art such as immunocytochemistry, immunohistochemistry, by medical imaging techniques such as MRI, studying the neuron morphology and synaptic degeneration (by measuring density of proteins located in synapses) or by analyzing expression levels of several senescence and neurodegeneration markers. In this context, “decrease” (respectively “improvement”) means at least a detectable decrease (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The increase may be seen after at least one week, one month, six months, one year or more of treatment using a gene construct and/or an expression vector and/or a composition of the invention. Preferably, the increase is observed after a single administration. In some embodiments, the increase is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Alleviating a symptom may mean that the progression of a typical symptom (e.g. neuroinflammation, neurodegeneration, cognitive decline, memory loss, decreased learning capacity, synapse loss, tau phosphorylation) has been slowed down in an individual, in a cell, tissue or organ of said individual as assessed by a physician. A decrease of a typical symptom may mean a slowdown in progression of symptom development or a complete disappearance of symptoms. Symptoms, and thus also a decrease in symptoms, can be assessed using a variety of methods, to a large extent the same methods as used in diagnosis of neuroinflammation, neurodegeneration, cognitive decline, and diseases associated therewith, including clinical examination and routine laboratory tests. Clinical examination may include behavioral tests and cognitive tests. Laboratory tests may include both macroscopic and microscopic methods, molecular methods, radiographic methods such as X-rays, biochemical methods, immunohistochemical methods and others. Memory and learning may be assed in mice e.g. as described in the experimental part, e.g. by a novel object recognition test and/or a Morris water maze test. The alleviation of a symptom may be seen after at least one week, one month, six months, one year or more of treatment using a gene construct and/or an expression vector and/or a composition of the invention. Preferably, the alleviation is observed after a single administration. In some embodiments, the alleviation is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Improving a parameter may mean improving results after behavioral test, improving the expression of serum and CSF markers, improving the expression of apoptosis/neurogenesis cell markers, etc. The improvement of a parameter may be seen after at least one week, one month, six months, one year or more of treatment using a gene construct and/or an expression vector and/or a composition of the invention. Preferably, the improvement is observed after a single administration. In some embodiments, the improvement is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Within the context of gene constructs for use, expression vectors for use, pharmaceutical compositions for use, methods and uses according to the invention, a gene construct and/or an expression vector and/or a pharmaceutical composition as described herein preferably alleviates one or more symptom(s) of neuroinflammation, neurodegeneration and/or cognitive disorder, or a disease associated therewith, in an individual, in a cell, tissue or organ of said individual or alleviates one or more characteristic(s) or symptom(s) of a cell, tissue or organ of said individual.

A gene construct and/or an expression vector and/or a pharmaceutical composition as described herein is preferably able to alleviate a symptom or a characteristic of a patient or of a cell, tissue or organ of said patient if after at least one week, one month, six months, one year or more of treatment using a gene construct and/or an expression vector and/or a composition of the invention, said symptom or characteristic has decreased (e.g. is no longer detectable or has slowed down), as described herein.

A gene construct and/or an expression vector and/or a pharmaceutical composition and/or a medicament as described herein may be suitable for administration to a cell, tissue and/or an organ in vivo of individuals affected by or at risk of developing a me neuroinflammation, neurodegeneration and/or cognitive disorder, or a disease associated therewith, and may be administered in vivo, ex vivo or in vitro. Said gene construct and/or expression vector and/or pharmaceutical composition and/or medicament may be directly or indirectly administered to a cell, tissue and/or an organ in vivo of an individual affected by or at risk of developing neuroinflammation, neurodegeneration and/or cognitive disorder, or a disease associated therewith, and may be administered directly or indirectly in vivo, ex vivo or in vitro.

An administration mode may be intravenous, intramuscular, intrathecal, intraventricular, intraperitoneal, via inhalation, intranasal, intra-ocular and/or intraparenchymal administration. Preferred administration modes are intranasal, intraparenchymal and intra-CSF (via cisterna magna, intrathecal or intraventricular delivery) administration. Intra-CSF administration is most preferred. A preferred mode of administration, optionally a preferred mode of administration in humans, is intraventricular.

A gene construct and/or an expression vector and/or a composition and/or a medicament of the invention may be directly or indirectly administered using suitable means known in the art. Improvements in means for providing an individual or a cell, tissue, organ of said individual with a gene construct and/or an expression vector and/or a composition and/or a medicament of the invention are anticipated, considering the progress that has already thus far been achieved. Such future improvements may of course be incorporated to achieve the mentioned effect of the invention. A gene construct and/or an expression vector and/or a composition and/or a medicament can be delivered as is to an individual, a cell, tissue or organ of said individual. Depending on the disease or condition, a cell, tissue or organ of said individual may be as earlier described herein. When administering a gene construct and/or an expression vector and/or a composition and/or a medicament of the invention, it is preferred that such gene construct and/or expression vector and/or composition and/or medicament is dissolved in a solution that is compatible with the delivery method.

As encompassed herein, a therapeutically effective dose of a gene construct and/or an expression vector and/or a composition as mentioned above is preferably administered in a single and unique dose hence avoiding repeated periodical administration.

### General Information

Unless stated otherwise, all technical and scientific terms used herein have the same meaning as customarily and ordinarily understood by a person of ordinary skill in the art to which this invention belongs, and read in view of this disclosure.

### Sequence Identity/Similarity

In the context of the invention, a nucleic acid molecule such as a nucleic acid molecule encoding an insulin is represented by a nucleotide sequence which encodes a protein fragment or a polypeptide or a peptide or a derived peptide. In the context of the invention, an insulin protein fragment or a polypeptide or a peptide or a derived peptide is represented by an amino acid sequence.

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide. Throughout this application, each time one refers to a specific nucleotide sequence SEQ ID NO (take SEQ ID NO: X as example) encoding a given protein fragment or polypeptide or peptide or derived peptide, one may replace it by:

i. a nucleotide sequence comprising a nucleotide sequence that has at least 60% sequence identity with SEQ ID NO: X;

ii. a nucleotide sequence the sequence of which differs from the sequence of a nucleic acid molecule of (i) due to the degeneracy of the genetic code; or

iii. a nucleotide sequence that encodes an amino acid sequence that has at least 60% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide comprising an amino acid sequence that has at least 60% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art. For example, such activity is inducing the detectable expression of a nucleotide sequence operably linked to the promoter, such as the insulin coding sequence.

The terms “homology”, “sequence identity”, “identity” and the like are used interchangeably herein. Sequence identity is herein described as a relationship between two or more amino acid sequences (peptide or polypeptide or protein) or two or more nucleic acid sequences (polynucleotide), as determined by comparing the sequences. “Similarity” or “sequence similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004.

Sequence identity or similarity can be calculated based on the full length of two given SEQ ID NO's or on part thereof. In some embodiments, part thereof means at least 50%, 60%, 70%, 80%, 90%, 95% or 100% of both SEQ ID NO. In a preferred embodiment, sequence identity or similarity is determined by comparing the whole length of the sequences as identified herein. Unless otherwise indicated herein, identity or similarity with a given SEQ ID NO means identity or similarity based on the full length of said sequence (i.e. over its whole length or as a whole). In the art, “identity” also refers to the degree of sequence relatedness between amino acid or nucleotide sequences, as the case may be, as determined by the match between strings of such sequences.

Sequence identity or similarity can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity or similarity (as described below).

A global alignment is suitably used to determine sequence identity or similarity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters may be used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919).

Alternatively percentage similarity or identity may be determined by searching against public databases, using algorithms such as FASTA, BLAST, etc. Homologene may also be used (https://en.wikipedia.org/wiki/HomoloGene), preferably without modifying the default parameters. Thus, the nucleotide and amino acid sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules, preferably encoding insulin, of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, a person of skill in the art may also take into account so-called conservative amino acid substitutions.

As used herein, “conservative” amino acid substitutions refer to the interchangeability of residues having similar side chains. Examples of classes of amino acid residues for conservative substitutions are shown below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes are as follows:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, |
| | T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, |
| | R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

### Gene or Coding Sequence

A “gene” is a sequence of nucleotides in DNA or RNA that codes for a molecule that has a function. A nucleotide sequence may comprise “non-coding sequence” as well as “coding sequence”. The coding region of a “gene”, also known as the CDS (from coding sequence), is that portion of a gene's DNA or RNA that codes for protein. Examples of non-coding sequences are promoters and microRNA target sequences as described elsewhere herein. The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site. A chimeric or recombinant gene (such as a chimeric or recombinant insulin gene) is a gene not normally found in nature, such as a gene in which for example the promoter is not associated in nature with part or all of the transcribed DNA region. “Expression of a gene” refers to the process wherein a DNA region which is operably linked to appropriate regulatory regions, particularly a promoter, is transcribed into an RNA, which is biologically active, e.g. which is capable of being translated into a biologically active protein or peptide.

A “transgene” is herein described as a gene or a coding sequence or a nucleic acid molecule represented by a nucleotide sequence (i.e. a molecule encoding an insulin) that has been newly introduced into a cell, i.e. a gene that may be present but may normally not be expressed or expressed at an insufficient level in a cell. In this context, “insufficient” means that although said insulin is expressed in a cell, a condition and/or disease as described herein could still be developed. In this case, the invention allows the over-expression of an insulin. The transgene may comprise sequences that are native to the cell, sequences that naturally do not occur in the cell and it may comprise combinations of both. A transgene may contain sequences coding for an insulin and/or additional proteins as earlier identified herein that may be operably linked to appropriate regulatory sequences for expression of the sequences coding for an insulin in the cell. Preferably, the transgene is not integrated into the host cell's genome.

### Promoter

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally or otherwise regulated, e.g. by the application of a chemical inducer.

A “ubiquitous promoter” is active in substantially all tissues, organs and cells of an organism. In some embodiments, a ubiquitous promoter drives expression in at least 5, 6, 7, 8, 9, 10 or more different types of tissues, organs and/or cells.

An “organ-specific” or “tissue-specific” promoter is a promoter that is active in a specific type of organ or tissue, respectively. Organ-specific and tissue-specific promoters regulate expression of one or more genes (or coding sequence) primarily in one organ or tissue, but can allow detectable level (“leaky”) expression in other organs or tissues as well. Leaky expression in other organs or tissues means at least one-fold, at least two-fold, at least three-fold, at least four-fold, at least five-fold, at least six-fold, at least seven-fold, at least eight-fold, at least nine-fold or at least ten-fold lower, but still detectable expression as compared to the organ-specific or tissue-specific expression, as evaluated on the level of the mRNA or the protein by standard assays known to a person of skill in the art (e.g. qPCR, Western blot analysis, ELISA). The maximum number of organs or tissues where leaky expression may be detected is five, six, seven or eight.

A “CNS- and/or brain-specific promoter” is a promoter that is capable of initiating transcription in the CNS and/or brain, whilst still allowing for any leaky expression in other (maximum five, six, seven or eight) organs and parts of the body. Transcription in the CNS and/or brain can be detected in relevant areas, such as the CNS and/or brain and/or hypothalamus and/or cortex and/or hippocampus and/or cerebellum and/or olfactory bulb, and cells, such as neurons and/or glial cells.

In the context of the invention, CNS- and/or brain-specific promoters may be promoters that are capable of driving the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of insulin in the CNS and/or the brain as compared to other organs or tissues. Other organs or tissues may be the liver, pancreas, adipose tissue, skeletal muscle, heart, kidney, colon, hematopoietic tissue, lung, ovary, spleen, stomach, testis and others. Preferably, other organs are the liver and/or the heart. Other organs may also be skeletal muscle. A CNS- and/or brain-specific promoter, as used herein, also encompasses promoters directing expression in a specific region or cellular subset of the CNS and/or brain. Accordingly, CNS- and/or brain specific promoters may also be selected from a hippocampus-specific promoter, a cerebellum-specific promoter, a cortex-specfific promoter, a hypothalamus-specific promoter and/or an olfactory bulb-specific promoter, or any combination thereof. Expression may be assessed using techniques such as qPCR, Western blot analysis or ELISA as described under the section entitled “general information”.

Throughout the application, where CNS- and/or brain-specific is mentioned in the context of expression, cell-type specific expression of the cell type(s) making up the CNS and/or the brain is also envisaged, respectively.

### Operably Linked

As used herein, the term “operably linked” refers to a linkage of polynucleotide elements in a functional relationship. A nucleic acid is “operably linked” when it is placed into a functional relationship with another nucleic acid molecule. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. Linking can be accomplished by ligation at convenient restriction sites or at adapters or linkers inserted in lieu thereof, or by gene synthesis, or any other method known to a person skilled in the art.

microRNA

As used herein, “microRNA” or “miRNA” or “miR” has its customary and ordinary meaning as understood by one of skill in the art in view of this disclosure. A microRNA is a small non-coding RNA molecule found in plants, animals and some viruses, that may function in RNA silencing and post-transcriptional regulation of gene expression. A target sequence of a microRNA may be denoted as “miRT”. For example, a target sequence of microRNA-1 or miRNA-1 or miR-1 may be denoted as miRT-1.

### Proteins and Amino Acids

The terms “protein” or “polypeptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

### Gene Constructs

Gene constructs as described herein could be prepared using any cloning and/or recombinant DNA techniques, as known to a person of skill in the art, in which a nucleotide sequence encoding said insulin is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl.

Acad. Sci. 82:488 (describing site directed mutagenesis) and Roberts et al. (1987) Nature 328:731-734 or Wells, J. A., et al. (1985) Gene 34: 315 (describing cassette mutagenesis).

### Expression Vectors

The phrase “expression vector” or “vector” generally refers to a nucleotide sequence that is capable of effecting expression of a gene or a coding sequence in a host compatible with such sequences. An expression vector carries a genome that is able to stabilize and remain episomal in a cell. Within the context of the invention, a cell may mean to encompass a cell used to make the construct or a cell wherein the construct will be administered. Alternatively, a vector is capable of integrating into a cell's genome, for example through homologous recombination or otherwise.

These expression vectors typically include at least suitable promoter sequences and optionally, transcription termination signals. An additional factor necessary or helpful in effecting expression can also be used as described herein. A nucleic acid or DNA or nucleotide sequence encoding an insulin is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect (e.g., Sf9), yeast, fungi or other eukaryotic cell lines.

A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been described herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, DNA sequences that are operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis, or any other method known to a person skilled in the art.

The selection of an appropriate promoter sequence generally depends upon the host cell selected for the expression of a DNA segment. Examples of suitable promoter sequences include prokaryotic and eukaryotic promoters well known in the art (see, e.g. Sambrook and Russell, 2001, supra). A transcriptional regulatory sequence typically includes a heterologous enhancer or promoter that is recognized by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment. In most cases, the replication system is only functional in the cell that is used to make the vector (bacterial cell as E. coli). Most plasmids and vectors do not replicate in the cells infected with the vector. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, insect cells, e.g. Sf9 cells, mammalian cells, e.g., CHO cells, and bacterial cells, e.g., E. coli. A cell may thus be a prokaryotic or eukaryotic host cell. A cell may be a cell that is suitable for culture in liquid or on solid media.

Alternatively, a host cell is a cell that is part of a multicellular organism such as a transgenic plant or animal.

### Viral Vector

A viral vector or a viral expression vector or a viral gene therapy vector is a vector that comprises a gene construct as described herein.

A viral vector or a viral gene therapy vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein. Additional references describing gene therapy vectors are Naldini 2015, Nature 5526(7573):351-360; Wang et al. 2019 Nat Rev Drug Discov 18(5):358-378; Dunbar et al. 2018 Science 359(6372); Lukashey et al. 2016 Bioschemistry (Mosc) 81(7):700-708.

A particularly suitable gene therapy vector includes an adenoviral and adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications (Russell, 2000, J. Gen. Virol. 81: 2573-2604; Goncalves, 2005, Virol J. 2(1):43) as indicated above. AAV vectors are even more preferred since they are known to result in very stable long-term expression of transgene expression (up to 9 years in dog (Niemeyer et al, Blood. 2009 Jan. 22; 113(4):797-806) and ˜10 years in human (Buchlis, G. et al., Blood. 2012 Mar. 29; 119(13):3038-41). Preferred adenoviral vectors are modified to reduce the host response as reviewed by Russell (2000, supra). Gene therapy methods using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34.

Another suitable gene therapy vector includes a retroviral vector. A preferred retroviral vector for application in the present invention is a lentiviral based expression construct. Lentiviral vectors have the ability to infect and to stably integrate into the genome of dividing and non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Other suitable gene therapy vectors include an adenovirus vector, a herpes virus vector, a polyoma virus vector or a vaccinia virus vector.

### Adeno-associated Virus Vector (AAV Vector)

The terms “adeno associated virus”, “AAV virus”, “AAV virion”, “AAV viral particle” and “AAV particle”, used as synonyms herein, refer to a viral particle composed of at least one capsid protein of AAV (preferably composed of all capsid protein of a particular AAV serotype) and an encapsulated polynucleotide of the AAV genome. If the particle comprises a heterologous polynucleotide (i.e. a polynucleotide different from a wild-type AAV genome, such as a transgene to be delivered to a mammalian cell) flanked by AAV inverted terminal repeats, then they are typically known as a “AAV vector particle” or “AAV viral vector” or “AAV vector”. AAV refers to a virus that belongs to the genus Dependovirus family Parvoviridae. The AAV genome is approximately 4.7 Kb in length and it consists of single strand deoxyribonucleic acid (ssDNA) that can be positive or negative detected. The invention also encompasses the use of double stranded AAV also called dsAAV or scAAV. The genome includes inverted terminal repeats (ITR) at both ends of the DNA strand, and two open reading frames (ORFs): rep and cap. The frame rep is made of four overlapping genes that encode proteins Rep necessary for the AAV lifecycle. The frame cap contains nucleotide sequences overlapping with capsid proteins: VP1, VP2 and VP3, which interact to form a capsid of icosahedral symmetry (see Carter and Samulski, Int J Mol Med 2000, 6(1):17-27, and Gao et al, 2004).

A preferred viral vector or a preferred gene therapy vector is an AAV vector. An AAV vector as used herein preferably comprises a recombinant AAV vector (rAAV vector). A “rAAV vector” as used herein refers to a recombinant vector comprising part of an AAV genome encapsidated in a protein shell of capsid protein derived from an AAV serotype as explained herein. Part of an AAV genome may contain the inverted terminal repeats (ITR) derived from an adeno-associated virus serotype, such as AAV1, AAV2, AAV3, AAV4, AAV5 and others. Preferred ITRs are those of AAV2 which are represented by sequences comprising, consisting essentially of, or consisting of SEQ ID NO: 35 (5′ ITR) and SEQ ID NO: 36 (3′ ITR). The invention also preferably encompasses the use of a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 35 as 5′ ITR and a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 36 as 3′ ITR.

Protein shell comprised of capsid protein may be derived from any AAV serotype. A protein shell may also be named a capsid protein shell. rAAV vector may have one or preferably all wild type AAV genes deleted, but may still comprise functional ITR nucleotide sequences. Functional ITR sequences are necessary for the replication, rescue and packaging of AAV virions. The ITR sequences may be wild type sequences or may have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with wild type sequences or may be altered by for example by insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the rAAV vector genome ITR.

A nucleic acid molecule represented by a nucleotide sequence of choice, preferably encoding an insulin, is preferably inserted between the rAAV genome or ITR sequences as identified above, for example an expression construct comprising an expression regulatory element operably linked to a coding sequence and a 3′ termination sequence. Said nucleic acid molecule may also be called a transgene.

“AAV helper functions” generally refers to the corresponding AAV functions required for rAAV replication and packaging supplied to the rAAV vector in trans. AAV helper functions complement the AAV functions which are missing in the rAAV vector, but they lack AAV ITRs (which are provided by the rAAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73(2): 1309-1319) or U.S. Pat. NO. 5,139,941, incorporated herein by reference. The AAV helper functions can be supplied on an AAV helper construct. Introduction of the helper construct into the host cell can occur e.g. by transformation, transfection, or transduction prior to or concurrently with the introduction of the rAAV genome present in the rAAV vector as identified herein. The AAV helper constructs of the invention may thus be chosen such that they produce the desired combination of serotypes for the rAAV vector's capsid protein shell on the one hand and for the rAAV genome present in said rAAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via plasmids, as described in U.S. Pat. No. 6,531,456 incorporated herein by reference.

“Transduction” refers to the delivery of an insulin into a recipient host cell by a viral vector. For example, transduction of a target cell by a rAAV vector of the invention leads to transfer of the rAAV genome contained in that vector into the transduced cell. “Host cell” or “target cell” refers to the cell into which the DNA delivery takes place, such as the muscle cells of a subject. AAV vectors are able to transduce both dividing and non-dividing cells.

### Production of an AAV Vector

The production of recombinant AAV (rAAV) for vectorizing transgenes have been described previously. See Ayuso E, et al., Curr. Gene Ther. 2010; 10:423-436, Okada T, et al., Hum. Gene Ther. 2009; 20:1013-1021, Zhang H, et al., Hum. Gene Ther. 2009; 20:922-929, and Virag T, et al., Hum. Gene Ther. 2009; 20:807-817. These protocols can be used or adapted to generate the AAV of the invention. In one embodiment, the producer cell line is transfected transiently with the polynucleotide of the invention (comprising the expression cassette flanked by ITRs) and with construct(s) that encodes rep and cap proteins and provides helper functions. In another embodiment, the cell line supplies stably the helper functions and is transfected transiently with the polynucleotide of the invention (comprising the expression cassette flanked by ITRs) and with construct(s) that encodes rep and cap proteins. In another embodiment, the cell line supplies stably the rep and cap proteins and the helper functions and is transiently transfected with the polynucleotide of the invention. In another embodiment, the cell line supplies stably the rep and cap proteins and is transfected transiently with the polynucleotide of the invention and a polynucleotide encoding the helper functions. In yet another embodiment, the cell line supplies stably the polynucleotide of the invention, the rep and cap proteins and the helper functions.

Methods of making and using these and other AAV production systems have been described in the art. See Muzyczka N, et al., U.S. Pat. No. 5,139,941, Zhou X, et al., U.S. Pat. No. 5,741,683, Samulski R, et al., U.S. Pat. No. 6,057,152, Samulski R, et al., U.S. Pat. No. 6,204,059, Samulski R, et al., U.S. Pat. No. 6,268,213, Rabinowitz J, et al., U.S. Pat. No. 6,491,907, Zolotukhin S, et al., U.S. Pat. No. 6,660,514, Shenk T, et al., U.S. Pat. No. 6,951,753, Snyder R, et al., U.S. Pat. No. 7,094,604, Rabinowitz J, et al., U.S. Pat. No. 7,172,893, Monahan P, et al., U.S. Pat. No. 7,201,898, Samulski R, et al., U.S. Pat. No. 7,229,823, and Ferrari F, et al., U.S. Pat. No. 7,439,065.

The rAAV genome present in a rAAV vector comprises at least the nucleotide sequences of the inverted terminal repeat regions (ITRs) of one of the AAV serotypes (preferably the ones of serotype AAV2 as disclosed earlier herein), or nucleotide sequences substantially identical thereto or nucleotide sequences having at least 60% identity thereto, and nucleotide sequence encoding an insulin (under control of a suitable regulatory element) inserted between the two ITRs. A vector genome requires the use of flanking 5′ and a 3′ ITR sequences to allow for efficient packaging of the vector genome into the rAAV capsid.

The complete genome of several AAV serotypes and corresponding ITR has been sequenced (Chiorini et al. 1999, J. of Virology Vol. 73, No. 2, p 1309-1319). They can be either cloned or made by chemical synthesis as known in the art, using for example an oligonucleotide synthesizer as supplied e.g. by Applied Biosystems Inc. (Fosters, Calif., USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the AAV sequence between the ITRs can be replaced with the desired nucleotide sequence.

Preferably, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

The rAAV genome as present in said rAAV vector further comprises a promoter sequence operably linked to the nucleotide sequence encoding an insulin.

A suitable 3′ untranslated sequence may also be operably linked to the nucleotide sequence encoding an insulin. Suitable 3′ untranslated regions may be those naturally associated with the nucleotide sequence or may be derived from different genes, such as for example the SV40 polyadenylation signal (SEQ ID NO: 37) and the rabbit β-globin polyadenylation signal (SEQ ID NO: 38).

### Expression

Expression may be assessed by any method known to a person of skill in the art. For example, expression may be assessed by measuring the levels of transgene expression in the liver on the level of the mRNA or the protein by standard assays known to a person of skill in the art, such as qPCR, Western blot analysis or ELISA.

Expression may be assessed at any time after administration of the gene construct, expression vector or composition as described herein.

In some embodiments herein, expression may be detected as soon as after 1 day, 2 days, 3 days, 4 days, 1 week, 2 weeks, 3 weeks, 4 weeks, 5 weeks, 6 weeks, 7 weeks, 8 weeks, 9, weeks or 10 weeks.

In some embodiments herein, expression may last at least 4 weeks 5 weeks, 6 weeks, 7 weeks, 8 weeks, 9, weeks, 10 weeks. 11 weeks, 12 weeks, 14 weeks, 16 weeks, 18 weeks, 20 weeks, 22 weeks, 24 weeks, 28 weeks, 32 weeks, 36 weeks, 40 weeks, 44 weeks, 48 weeks, 1 year, 2 years, 3 years, 4 years, 5 years, 6 years, 7 years, 8 years, 9 years, 10 years, 12 years, 15 years, 20 years or more. In other words, this means that expression can still be detected at 4 weeks 5 weeks, 6 weeks, 7 weeks, 8 weeks, 9, weeks, 10 weeks. 11 weeks, 12 weeks, 14 weeks, 16 weeks, 18 weeks, 20 weeks, 22 weeks, 24 weeks, 28 weeks, 32 weeks, 36 weeks, 40 weeks, 44 weeks, 48 weeks, 1 year, 2 years, 3 years, 4 years, 5 years, 6 years, 7 years, 8 years, 9 years, 10 years, 12 years, 15 years, 20 years or more after administration.

In some embodiments, this expression is detected after a single administration.

In the context of the invention, CNS- and/or brain- and/or hypothalamus and/or cortex- and/or hippocampus- and/or cerebellum- and/or olfactory bulb-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of insulin in the CNS and/or the brain and/or the hypothalamus and/or the cortex and/or the hippocampus and/or the cerebellum and/or the olfactory bulb as compared to other organs or tissues. Other organs or tissues may be the liver, pancreas, adipose tissue, skeletal muscle, heart, and others. In an embodiment, expression is not detectable in the liver, pancreas, adipose tissue, skeletal muscle and/or heart. In some embodiments, expression is not detectable in at least one, at least two, at least three, at least four or all organs selected from the group consisting of the liver, pancreas, adipose tissue, skeletal muscle and heart. Expression may be assessed as described above.

Throughout the application, where CNS- and/or brain- and/or hypothalamus and/or cortex- and/or hippocampus- and/or cerebellum- and/or olfactory bulb-specific is mentioned in the context of expression, cell-type specific expression of the cell type(s) making up the CNS and/or the brain and/or the hypothalamus and/or the cortex and/or the hippocampus and/or the cerebellum and/or the olfactory bulb is also envisaged, respectively.

### Administration

As used herein, “intra-CSF administration” means direct administration into the CSF, located in the subarachnoid space between the arachnoid and pia mater layers of the meninges surrounding the brain. Intra-CSF administration can be performed via intra-cisterna magna, intraventricular or intrathecal administration. As used herein, “intra-cisterna magna administration” means administration into the cisterna magna, an opening of the subarachnoid space located between the cerebellum and the dorsal surface of the medulla oblongata. As used herein, “intraventricular administration” means administration into the either of both lateral ventricles of the brain As used herein, “intrathecal administration” involves the direct administration into the CSF within the intrathecal space of the spinal column. As used herein, “intraparenchymal administration” means local administration directly into any region of the brain parenchyma. As used herein, “intranasal administration” means administration by way of the nasal structures.

In a preferred embodiment, gene constructs, expression vectors and compositions according to the invention are administered as a single dose.

### Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. For example, to suit the codon preference of mammalians, preferably of murine, canine or human expression hosts. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs). In some embodiments, codon-optimized sequences show at least 3%, 5%, 10%, 15%, 20%, 25%, 30%, 40%, 50%, 60%, 70%, 80%, 90%, 100% or more increase in transcription, RNA stability and/or translation.

### CNS and Brain

As used herein, “central nervous system” or “CNS” refers to the part of the nervous system that comprises the brain and the spinal cord, to which sensory impulses are transmitted and from which motor impulses pass out, and which coordinates the activity of the entire nervous system.

As used herein, “brain” refers to the central organ of the nervous system and consists of the cerebrum, the brainstem and the cerebellum. It controls most of the activities of the body, processing, integrating, and coordinating the information it receives from the sense organs, and making decisions as to the instructions sent to the rest of the body.

In particular, as used herein, ‘hypothalamus” refers to a region of the forebrain below the thalamus which coordinates both the autonomic nervous system and the activity of the pituitary, controlling body temperature, thirst, hunger, and other homeostatic systems, and involved in sleep and emotional activity. “Hippocampus”, as used herein, belongs to the limbic system and plays important roles in the consolidation of information from short-term memory to long-term memory, and in spatial memory that enables navigation. The hippocampus is located under the cerebral cortex (allocortical) and in primates in the medial temporal lobe. The “cortex” or “cerebral cortex”, as used herein, is the outer layer of neural tissue of the cerebrum of the brain, in humans and other mammals. It plays a key role in memory, attention, perception, awareness, thought, language, and consciousness. “Cerebellum”, as used herein, refers to a major feature in the hindbrain of all vertebrates. In humans, it plays an important role in motor control. It may also be involved in some cognitive functions such as attention and language as well as in regulating fear and pleasure responses. “Olfactory bulb”, as used herein, refers to an essential structure in the olfactory system (the system devoted to the sense of smell. The olfactory bulb sends information to be further processed in the amygdala, the orbitofrontal cortex (OFC) and the hippocampus where it plays a role in emotion, memory and learning.

### Memory

Memory is generally understood to be the faculty of the brain by which data or information is encoded, stored, and retrieved when needed. Different types or memory have been described. One possible distinction involves sensory memory, short-term memory and long-term memory. Sensory memory holds sensory information less than one second after an item is perceived. Short-term (also known as working memory) memory allows recall for a period of several seconds to a minute, typically without rehearsal. Long-term memory, on the contrary, can store much larger quantities of information for a potentially unlimited duration (up to a whole life span).

Another distinction involves procedural memory (or implicit memory) and explicit memory (or declarative memory). Implicit memory is not based on the conscious recall of information, but on implicit learning, i.e. remembering how to do something. Explicit (or declarative) memory is the conscious, intentional recollection of factual information, previous experiences, and concepts.

A distinction can also be made between recall memory and recognition memory. Recognition refers to our ability to “recognize” an event or piece of information as being familiar, while recall designates the retrieval of related details from memory.

Spatial memory is a form of memory responsible for the recording of information about one's environment and spatial orientation.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a gene construct, expression vector or a composition as described herein may comprise additional component(s) than the ones specifically identified, said additional component(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. In the absence of any contrary consideration, the word “about” or “approximately” when used in association with a numerical value (e.g. about 10) preferably means that the value may be the given value (of 10) more or less 1% of the value.

As used herein, the term “and/or” indicates that one or more of the stated cases may occur, alone or in combination with at least one of the stated cases, up to with all of the stated cases.

Each embodiment described herein may be combined together with any other embodiment described herein, unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

A person of skill in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Expression of hlns in the brain of SAMP8 mice. The expression levels of human insulin (hlns) coding sequence were measured by RTqPCR in Hypothalamus, Cortex, Hippocampus and Cerebellum of SAMP8 mice, and normalized with Rplp0 values. Analyses were performed 14 weeks after intra-CSF administration of 5×10¹⁰ vg/mouse of AAV9-CAG-hlns-dmiRT vectors. Results are expressed as the mean±SEM, n=9 animals/group. ND, non-detected.

FIG. 2. Reduction of brain inflammation in SAMP8 mice treated with AAV9-hlns vectors. Expression levels of inflammatory molecules (Nfkb, II1b and II6) were measured by RTqPCR in Hypothalamus, Hippocampus and Cerebellum of SAMP8 mice, and normalized with Rplp0 values. Analyses were performed 14 weeks after intra-CSF administration of 5×10¹⁰ vg/mouse of AAV9-CAG-hlns-dmiRT vectors. Results are expressed as the mean±SEM, n=9 animals/group. *p<0.05, **p<0.01 vs non-treated mice. Nfkb, nuclear factor kappa B; II1b. interleukin-1 beta; II6, interleukin-6.

FIG. 3. Increased expression of astrocyte markers in the brain of SAMP8 mice treated with AAV9-hlns vectors. Expression levels of astrocyte markers (Gfap and S100b) were measured by RTqPCR in Hypothalamus, Cortex, Hippocampus and Cerebellum of SAMP8 mice, and normalized with Rplp0 values. Analyses were performed 14 weeks after intra-CSF administration of 5×10¹⁰ vg/mouse of AAV9-CAG-hlns-dmiRT vectors. Results are expressed as the mean±SEM, n=9 animals/group. *p<0.05, **p<0.01 vs non-treated mice. Gfap, glial fibrillary acidic protein; S100b, calcium-binding protein B.

FIG. 4. Increased neurogenesis in the brain of AAV9-hlns-treated SAMP8 mice. The expression levels of neurogenic markers (Dcx, Ncam and Sox2) were measured by RTqPCR in Cortex of SAMP8 mice, and normalized with Rplp0 values. Analyses were performed 14 weeks after intra-CSF administration of 5×10¹⁰ vg/mouse of AAV9-CAG-hlns-dmiRT vectors. Results are expressed as the mean±SEM, n=9 animals/group. *p<0.05 vs non-treated mice. Dcx, doublecortin; Ncam, neural cell adhesion molecule; Sox2, sex determining region Y box 2.

FIG. 5. Expression of hlns in the brain of db/db mice. The expression levels of human insulin (hlns) coding sequence were measured by RTqPCR in Hypothalamus, Cortex, Hippocampus and Cerebellum of db/db mice, and normalized with Rplp0 values. Analyses were performed 12 weeks after intra-CSF administration of 5×10¹⁰ vg/mouse of AAV9-CAG-hlns-dmiRT vectors. Results are expressed as the mean±SEM, n=9 animals/group. ND, non-detected.

FIG. 6. Reduction of brain inflammation in db/db mice treated with AAV9-hlns vectors. The expression levels of inflammatory molecules (Nfkb, II1b and II6) were measured by RTqPCR in Hypothalamus, Hippocampus and Cerebellum of db/db mice, and normalized with Rplp0 values. Analyses were performed 12 weeks after intra-CSF administration of 5×10¹⁰ vg/mouse of AAV9-CAG-hlns-dmiRT vectors. Results are expressed as the mean±SEM, n=9 animals/group. **p<0.01 vs non-treated mice. Nfkb, nuclear factor kappa B; II1b, interleukin-1 beta; II6, interleukin-6.

FIG. 7. Increased expression of astrocyte markers in the brain of db/db mice treated with AAV9-hlns vectors. Expression levels of astrocyte markers (Gfap and S100b) were measured by RTqPCR in Hypothalamus, Cortex, Hippocampus and Cerebellum of db/db mice, and normalized with Rplp0 values. Analyses were performed 12 weeks after intra-CSF administration of 5×10¹⁰ vg/mouse of AAV9-CAG-hlns-dmiRT vectors. Results are expressed as the mean±SEM, n=9 animals/group. *p<0.05, **p<0.01 vs non-treated mice. Gfap, glial fibrillary acidic protein; S100b, calcium-binding protein B.

FIG. 8. Transduction of brain after intra-CSF administration of AAV1-hlns, AAV2-hlns and AAV9-hlns vectors. (A) Vector genome copy numbers were determined in DNA isolated from Hypothalamus, Cortex, Hippocampus and Cerebellum of wild-type mice three weeks after intra-CSF administration of 5×10¹⁰ vg/mouse of AAV1-CAG-hlns-dmiRT, AAV2-CAG-hlns-dmiRT or AAV9-CAG-hlns-dmiRT vectors, by quantitative PCR with primers specific for hlns. (B) The expression levels of the human insulin (hlns) were measured by RTqPCR in Hypothalamus, Cortex, Hippocampus and Cerebellum of the same animals. Results were normalized with Rplp0 values. Results are expressed as the mean±SEM, n=5 animals/group. ND, non-detected.

FIG. 9. Expression of hlns in the brain of SAMP8 mice. The expression levels of human insulin (hlns) coding sequence were measured by RTqPCR in Hypothalamus, Cortex, Hippocampus, Cerebellum and Olfactory Bulb of SAMP8 mice, and normalized with Rplp0 values. Analyses were performed 34 weeks after intra-CSF administration of 5×10¹⁰ vg/mouse of AAV1-CAG-hlnsAsp and AAV1-CAG-hlnsWt vectors, respectively. Results are expressed as the mean±SEM, n=5 animals/group. ND, non-detected.

FIG. 10. Amelioration of short-term and long-term memory in SAMP8 mice after intra-CSF gene therapy with AAV1-CAG-hlnsWt and AAV1-CAG-hlnsAsp vectors. (A) Short-term and (B) long-term discrimination index was measured during a novel object recognition test in SAMR1 non-treated, SAMP8 non-treated, SAMP8 AAV1-CAG-hlnsWt-treated and SAMP8 AAV1-CAG-hlnsAsp-treated mice, at 33 weeks of age, and calculated as explained in the General Procedures of the Examples. Results are expressed as the mean±SEM, n=5 animals/group. *p<0.05, **p<0.01 vs SAMP8 non-treated mice.

FIG. 11. Amelioration of learning capacity in SAMP8 mice after intra-CSF gene therapy with AAV1-CAG-hlnsWt. The learning capacity was measured during a Morris Water Maze test in SAMR1 non-treated, SAMP8 non-treated and SAMP8 AAV1-CAG-hlnsWt-treated mice, at 39 weeks of age, and calculated as explained in the General Procedures of the Examples. Results are expressed as the mean±SEM, n=5 animals/group.

### EXAMPLES

To study the effects of insulin on the brain when overexpressed in this organ by using AAV vectors. Three different experiments have been performed:
Treatment of SAMP8 mice with AAV9-lns. Dose used: 5×10¹⁰ vg/mouse (Example 1).
Treatment of db/db mice with AAV9-lns. Dose used: 5×10¹° vg/mouse (Example 2).
Treatment of SAMP8 mice with AAV1-CAG-hlnsAsp and AAV1-CAG-hlnsWt. Dose used: 5×10¹⁰ vg/mouse (Example 5).

Moreover, we also examined brain transduction efficiency by AAV1-hlns, AAV2-hlns and AAV9-hlns vectors after intra-CSF administration of wild-type mice (Example 3).

### General Procedures to the Examples

### Subject Characteristics

Male SAMP8/TaHsd (SAMP8), BKS.Cg-+Lepr^{db}/+Lepr^{db} OlaHsd (db/db), and C57BI/6J (wild-type) mice were used. For example 5, SAMR1/TaHsd (SAMR1) were used. Mice were fed ad libitum with a standard diet (2018S Teklad Global Diets®, Harlan Labs., Inc., Madison, Wis., US) and kept under a light-dark cycle of 12 h (lights on at 8:00 a.m.) and stable temperature (22° C.±2). For tissue sampling, mice were anesthetized by means of inhalational anesthetic isoflurane (IsoFlo®, Abbott Laboratories, Abbott Park, Ill., US) and decapitated. Tissues of interest were excised and kept at −80° C. until analysis. All experimental procedures were approved by the Ethics Committee for Animal and Human Experimentation of the Universitat Autònoma de Barcelona.

### Recombinant AAV Vectors

Single-stranded AAV vectors of serotype 1, 2 and 9 were produced by triple transfection of HEK293 cells according to standard methods (Ayuso, E. et al., 2010. Curr Gene Ther. 10(6):423-36). For examples 1-3, cells were cultured in 10 roller bottles (850 cm², flat; Corning™, Sigma-Aldrich Co., Saint Louis, Mo., US) in DMEM 10% FBS to 80% confluence and co-transfected by calcium phosphate method with a plasmid carrying the expression cassette flanked by the AAV2 ITRs (SEQ ID NO: 40), a helper plasmid carrying the AAV2 rep gene and the AAV of serotype 1, 2 or 9 cap gene, respectively, and a plasmid carrying the adenovirus helper functions. The transgene used was the human insulin coding-sequence (SEQ ID NO: 46) driven by the early enhancer/chicken beta actin (CAG) promoter (SEQ ID NO: 22), with the addition of four tandem repeats of the miRT-122a sequence (5′CAAACACCATTGTCACACTCCA3′, SEQ ID NO: 7) and four tandems repeats of the miRT-1 sequence (5′TTACATACTTCTTTACATTCCA3′, SEQ ID NO: 8) cloned in the 3′ untranslated region of the expression cassette. For example 5, cells were co-transfected with a plasmid carrying the expression cassette flanked by the AAV2 ITRs (SEQ ID NO: 49 or SEQ ID NO: 50), a helper plasmid carrying the AAV2 rep gene and the AAV of serotype 1, and a plasmid carrying the adenovirus helper functions. The transgene used was either the human insulin aspartic coding-sequence containing the furin cleaving sites (SEQ ID NO: 46) or the human insulin wild-type coding-sequence containing the furin cleaving sites (SEQ ID NO: 45), respectively, driven by the early enhancer/chicken beta actin (CAG) promoter (SEQ ID NO: 22). AAVs were purified with an optimized method based on a polyethylene glycol precipitation step and two consecutive cesium chloride (CsCl) gradients. This second-generation CsCl-based protocol reduced empty AAV capsids and DNA and protein impurities dramatically (Ayuso, E. et al., 2010. Curr Gene Ther. 10(6):423-36). Purified AAV vectors were dialyzed against PBS, filtered and stored at −80° C. Titers of viral genomes were determined by quantitative PCR following the protocol described for the AAV2 reference standard material using linearized plasmid DNA as standard curve (Lock M., et al., Hum. Gene Ther. 2010; 21:1273-1285). The vectors were constructed according to molecular biology techniques well known in the art.

### In Vivo Intra-CSF Administration of AAV Vectors

Mice were anesthetized with an intraperitoneal injection of ketamine (100 mg/kg) and xylazine (10 mg/kg), and the skin of the posterior part of the head, from behind the ears to approximately between the scapulas, was shaved and rinsed with ethanol. Mice were held in prone position, with the head at a slightly downward inclination. A 2-mm rostro-caudal incision was made to introduce a Hamilton syringe at an angle of 45-55° into the cisterna magna, between the occiput and the C1-vertebra and 5 μl of vector dilution was administered. Given that the CNS is the main target compartment for vector delivery, mice were dosed with the same number of vector genomes (vg)/mouse irrespective of body weight (5×10¹⁰ vg/mice).

### RNA Analysis

Total RNA was obtained from hypothalamus, cortex, hippocampus, cerebellum and olfactory bulb using Tripure isolation reagent (Roche Diagnostics Corp., Indianapolis, Ind., US), and RNeasy Mini Kit or RNeasy Micro Kit for hippocampus samples (Qiagen NV, Venlo, NL). In order to eliminate the residual viral genomes, total RNA was treated with DNasel (Qiagen NV, Venlo, NL). For RT-PCR analysis, 1 μg of RNA samples was reverse-transcribed using Transcriptor First Strand cDNA Synthesis Kit (04379012001, Roche, Calif., USA). Real-time quantitative PCR was performed in the LightCycler 480 II (Roche, Mannheim, Germany) using TB Green Premix Ex TaqII (Takara Bio Europe, France). Data was normalized with Rplp0 values and analyzed as previously described (Pfaffl, M., Nucleic Acids Res. 2001; 29(9):e45).

### Vector Biodistribution

Hypothalamus, cortex, hippocampus, cerebellum and olfactory bulb were digested overnight in Proteinase K (0.2 mg/mL). Total DNA was isolated with the MasterPure DNA Purification Kit (Epicenter Biotechnologies, Madison, Wis., US). Vector genome copy number was determined in 20 ng of genomic DNA by TaqMan qPCR with primers and probes specific for human insulin. Vector genomes per sample were interpolated from a standard curve built by serial dilutions of linearized plasmids bearing the target sequence spiked into 20 ng of non-transduced genomic DNA.

### Novel Object Recognition Test

The novel object recognition tests were conducted in the open field box. Open-field test was used to acclimatize the mice to the box. The next day, to conduct the first trial, two identical objects (A and B) were placed in the upper right and upper left quadrants of the box, and then mice were placed backwards to both objects. After 10 min of exploration, mice were removed from the box, and allowed for 10 min break. In the second trial, one of the identical objects (A or B) was replaced with object C (new object). Mice were then put back into the box for a further 10 minutes of exploration to assess the short-term memory. 24-hours after the second trial, a third trial was performed replacing object C with a new object (D). Mice were then put back into the box for a further 10 minutes of exploration to assess the long-term memory. The amount of time animals spent exploring the novel object was recorded and evaluated using a video tracking system (SMART Junior; Panlab). The evaluation of novel object recognition test memory was expressed as a percentage of the discrimination ratio calculated according to the following formula: Discrimination ratio (%)=(N−F)/(N+F)×100%, where N represents the time spent in exploring the new object and F represents the time spent in exploring the same object.

### Morris Water Maze

Mice were trained to locate a submerged platform (diameter of 10 cm) in a water tank (diameter of 1 m, temperature 26-28° C.) by swimming and relying on external visible cues. Five-day procedure: familiarization (day 1), the mouse was placed on a visible platform and then allowed free for 30 seconds. Then, in two consecutive trials, mice were inserted in the maze from two different starting points. If the mouse did not reach the platform in 60 s, it was guided to the platform. Latency to reach the visible platform was measured; Training (days 2-4), the mouse was placed in different maze quadrants randomly. The latency to reach a hidden platform (positioned in the ‘correct’ quadrant) was measured in two trials per session for two sessions per day (1 h between sessions) with a cutoff of 60 s. Test (day 5), the last session of training was followed by a probe trial. The hidden platform was removed, the mouse was placed in the center of the pool, and the latency to cross the area where the platform was located was measured using a videotracking system (Viewpoint, France);

In Examples 1-4, the nucleotide sequence of H. sapiens insulin mutant His-B10-Asp with furin cleavage sites (hlnsAsp; SEQ ID NO: 46) is used. In Example 5 both the nucleotide sequence of H. sapiens insulin mutant His-B10-Asp with furin cleavage sites (hlnsAsp; SEQ ID NO: 46) and the nucleotide sequence of H. sapiens insulin wild type with furin cleavage sites (hlnswt; SEQ ID NO: 45) are used.

Genetically engineered furin endoprotease cleavage sites allow highly efficient production of mature insulin in non-pancreatic tissues; between 85-93% of the total insulin production is mature insulin (Gros et al., Hum Gene Ther. 1997 Dec. 10; 8(18):2249-59; Gros et al. Hum Gene Ther. 1999 May 1; 10(7):1207-17 and Riu et al. Diabetes. 2002 March; 51(3):704-11). Furin is known to be present in different brain areas (Foti et al. Gene Ther. 2009 November; 16(11):1314-1319), allowing the efficient production of mature insulin from a sequence containing furin cleavage sites in this organ.

### Example 1

### Decreased Neuroinflammation and Increased Neurogenesis in SAMP8 Mice by Intra-CSF Administration of AAV9-CAG-hlns-dmiRT Vectors

We evaluated the therapeutic potential of the AAV-mediated genetic engineering of the brain with insulin on neuroinflammation and neurogenesis. To this end, we used a senescence-accelerated mouse-prone 8 (SAMP8) mice, which is a widely used mouse model of senescence with age-related brain pathologies such as neuroinflammation (Takeda T., Neurochem. Res. 2009, 34(4):639-659; Griñan-Ferré C. et al. Mol. Neurobiol. 2016, 53(4):2435-2450).

Seven-week-old male SAMP8 mice were administered locally intra-CSF, through the cisterna magna, with 5×10¹⁰ vg/mouse of AAV9 vectors encoding human insulin under the control of the CAG ubiquitous promoter which included target sites of the liver-specific miR122 and the heart-specific miR1 (AAV9-CAG-hlns-dmiRT). As control, non-treated SAMP8 animals were used. At twenty-one weeks of age animals were euthanized and tissue samples were taken for analysis.

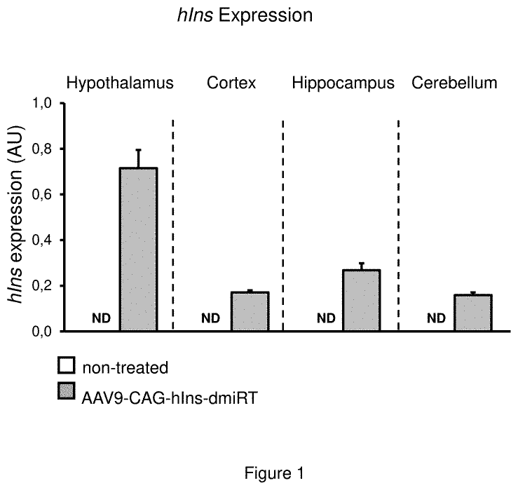

Intra-CSF administration of AAV9-CAG-hlns-dmiRT vectors mediated widespread overexpression of insulin in the brain, as evidenced by the increased expression levels of human insulin in different areas of the brain such as hypothalamus, cortex, hippocampus and cerebellum of SAMP8 mice (FIG. 1).

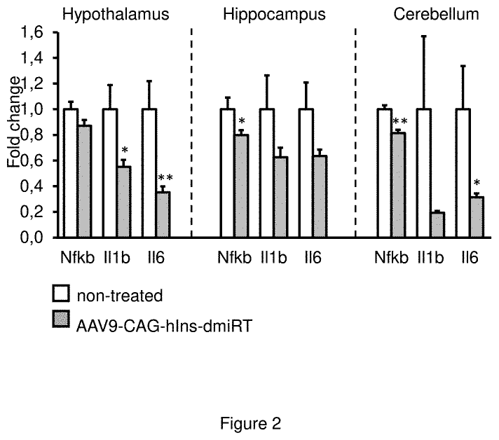

Neuroinflammation was analyzed through the expression of the pro-inflammatory molecules Nfkb, II1b and II6 in different areas of the brain. Noticeably, the expression of these pro-inflammatory molecules was decreased in all the brain areas analyzed (FIG. 2).

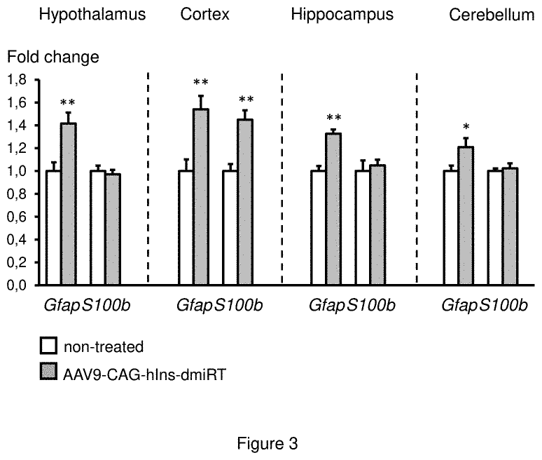

The expression of the astrocyte markers Gfap and S100b was analyzed. SAMP8 mice treated intra-CSF with AAV9-CAG-hlns-dmiRT vectors showed increased expression of Gfap in hypothalamus, cortex, hippocampus and cerebellum (FIG. 3) as well as increased expression of S100b in the cortex (FIG. 3). Astrocytes can secrete neurotransmitters and ATP, which are able to modulate activity of nearby neurons (Cai W. et al. Journal of Clinical Investigation 2018, 128(7):2914-2926). Therefore, the increase in astrocyte number could support neuronal activity and have anxiolytic and antidepressant effects. The decrease in pro-inflammatory markers accompanying the increase in astrocyte markers moreover indicates that the population of astrocytes that increases after the insulin gene therapy treatment is the population of “beneficial astrocytes”, also called “A2 astrocytes”.

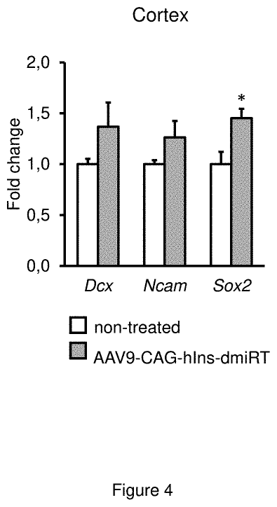

To study neurogenesis in SAMP8-treated mice, real time PCR of neurogenic markers was performed. Doublecortin (Dcx), neural cell adhesion molecule (Ncam) and sex determining region Y box 2 (Sox2) expression was increased in cortex of AAV9-CAG-hlns-dmiRT treated mice (FIG. 4).

### Example 2

### Decreased Neuroinflammation in db/db Mice by Intra-CSF Administration of AAV9-CAG-hlns-dmiRT Vectors

We evaluated the effects of insulin on neuroinflammation associated to obesity and/or diabetes in db/db mice. Db/db mice are a widely used genetic mouse model of obesity and diabetes, characterized by a deficit in leptin signalling. Moreover, these mice present not only inflammation in peripheral tissues such as adipose tissue and liver but also in the brain (Dey et al, J. Neuroimmmunol. 2014).

To this end, seven-week-old male db/db mice were administered intra-CSF, through the cisterna magna, with 5×10¹⁰vg/mouse of AAV9-CAG-hlns-dmiRT vectors. As control, non-treated db/db animals were used. At nineteen weeks of age, animals were euthanized and tissue samples were taken for analysis.

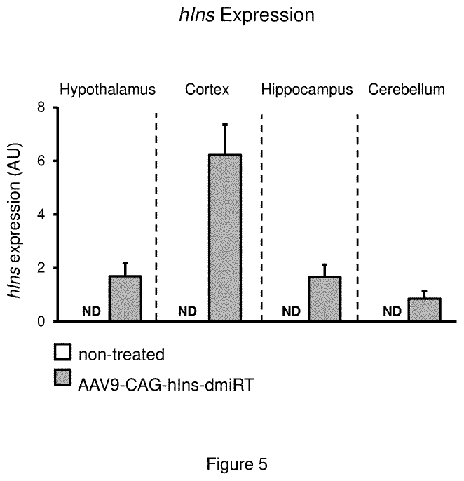

Similar to the observations made in SAMP8 mice, intra-CSF administration of AAV9-CAG-hlns-dmiRT vectors mediated robust overexpression of insulin in the hypothalamus, cortex, hippocampus and cerebellum of db/db mice (FIG. 5).

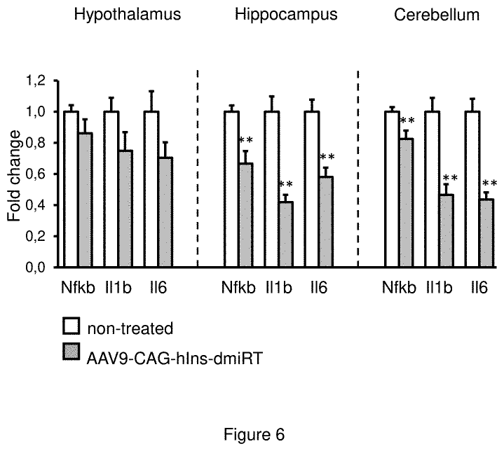

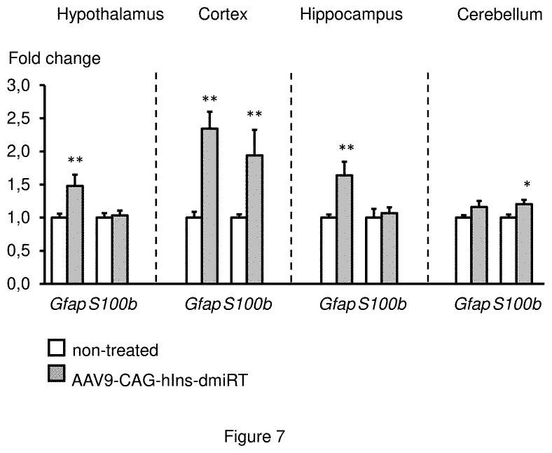

In db/db mice treated with the insulin-encoding vectors, the expression of the pro-inflammatory molecules Nfkb, II1b and II6 was decreased in all the brain areas analyzed (FIG. 6). Moreover, db/db-treated mice showed increased expression of the astrocyte marker Gfap in the hypothalamus, the cortex and the hippocampus, as well as increased expression of the astrocyte marker S100b in the cortex (FIG. 7). Astrocytes can secrete neurotransmitters and ATP, which are able to modulate activity of nearby neurons (Cai W. et al. Journal of Clinical Investigation 2018, 128(7):2914-2926). Therefore, the increase in astrocyte number could support neuronal activity and have anxiolytic and antidepressant effects. The decrease in pro-inflammatory markers accompanying the increase in astrocyte markers moreover indicates that the population of astrocytes that increases after the insulin gene therapy treatment is the population of “beneficial astrocytes”, also called “A2 astrocytes”.

### Example 3

### Brain Transduction after Intra-CSF Administration of AAV1-CAG-hlns-dmiRT, AAV2-CAG-hlns-dmiRT and AAV9-CAG-hlns-dmiRT Vectors

To examine whether different AAV serotypes were able to transduce the brain efficiently, wild-type mice were treated intra-CSF with 5×10¹⁰ vg/mice of AAV1, AAV2 and AAV9 vectors encoding a human insulin coding sequence under the control of the CAG ubiquitous promoter which included target sites of the liver-specific miR-122a and the heart-specific miR-1 (AAV1-CAG-hlns-dmiRT, AAV2-CAG-hlns-dmiRT and AAV9-CAG-hlns-dmiRT, respectively). As control, non-treated wild-type mice were used.

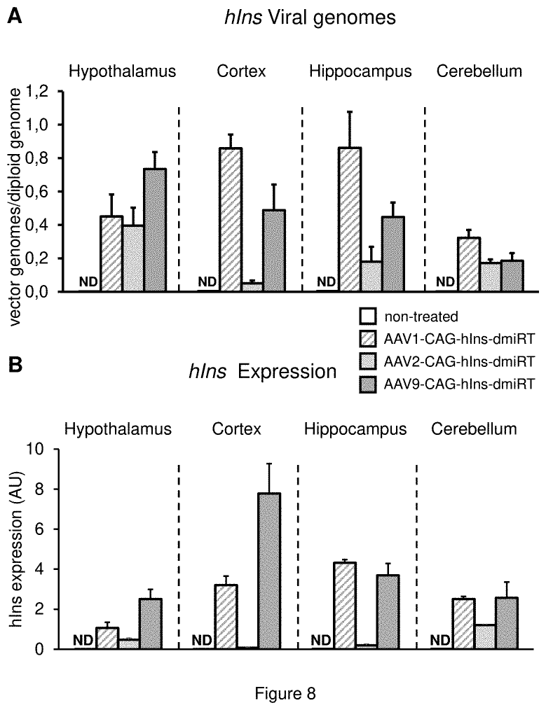

Three weeks after intra-CSF administration of the AAV vectors, brain samples were obtained and vector genomes copy number and human insulin expression were determined. As shown in FIG. 8, we found transduction of hypothalamus, cortex, hippocampus and cerebellum (FIG. 8A) and expression of hlns in the same brain areas (FIG. 8B), after AAV1, AAV2 and AAV9 intra-CSF administration.

### Example 4

### Intra-CSF Administration of AAV1-CAG-hlns Vectors in an Alzheimer's Disease Mouse Model

To evaluate the therapeutic potential of the AAV-mediated genetic engineering of the brain with insulin on Alzheimer's disease, the 3×Tg-AD (B6;129Tg(APPSwe,tauP301L)1Lfa Psen1^{tm1Mpm}) mouse model is used. The 3×Tg-AD is a widely used mouse model of Alzheimer's disease, homozygous for all three mutant alleles, homozygous for the Psen1 mutation and homozygous for the co-injected APPSwe and tauP301L transgenes (Belfiore, R., Aging Cell. 2019, 18(1):e12873) 3×Tg-AD mice are administered locally intra-CSF, through the cisterna magna, with 5×10¹⁰ vg/mouse of AAV1 vectors encoding human insulin under the control of the CAG ubiquitous promoter. As control, non-treated 3×Tg-AD animals are used. Several behavioural tests as Y-Maze, Open-Field and Morris Water Maze are performed in these mice. At 12 months of age, animals are euthanized and serum and tissue samples are taken for analysis.

Analysis of these samples include studies on neurogenesis (expression of neuronal markers such as Sox2, NeuN, and Dcx), neuroinflammation (expression of GFAP, Iba1 and several cytokine levels), levels of amyloid-beta (soluble amyloid and plaques), studies on synaptic degeneration (protein levels of synaptophysin and spine density), levels of tau phosphorylation.

### Example 5

### Amelioration of Short- and Long-Term Memory and Learning Capacity in SAMP8 Mice by Intra-CSF Administration of AAV1-CAG-hlnsAsp and AAV1-CAG-hlnsWt Vectors

We evaluated the therapeutic potential of the AAV-mediated genetic engineering of the brain with insulin on cognitive decline. To this end, we used the SAMP8 mouse model, which present cognitive decline by the age of 8-12 months (Miyamoto, M., Physiol Behav. 1986; 38(3):399-406; Markowska, A L., Physiol Behav. 1998; 64(1):15-26).

Seven-week-old male SAMP8 mice were administered locally intra-CSF, through the cisterna magna, with 5×10¹⁰ vg/mouse of AAV1 vectors encoding the human insulin aspartic or human insulin wild-type coding sequence under the control of the CAG ubiquitous promoter (AAV1-CAG-hlnsAsp and AAV1-CAG-hlnsWt vectors). As control, non-treated SAMP8 animals and non-treated SAM/resistant 1 (SAMR1) animals were used.

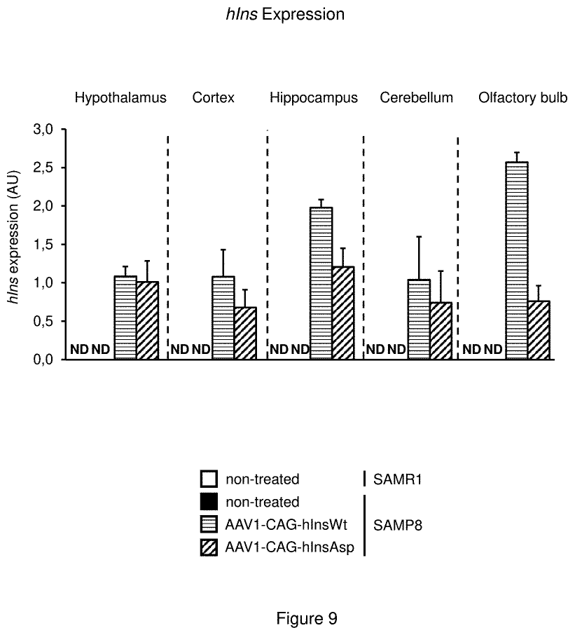

Intra-CSF administration of AAV1-CAG-hlnsAsp and AAV1-CAG-hlnswt vectors mediated widespread overexpression of insulin in the brain, as evidenced by the increased expression levels of human insulin in different areas of the brain such as hypothalamus, cortex, hippocampus, cerebellum and olfactory bulb of SAMP8 mice, at 41 weeks of age (FIG. 9).

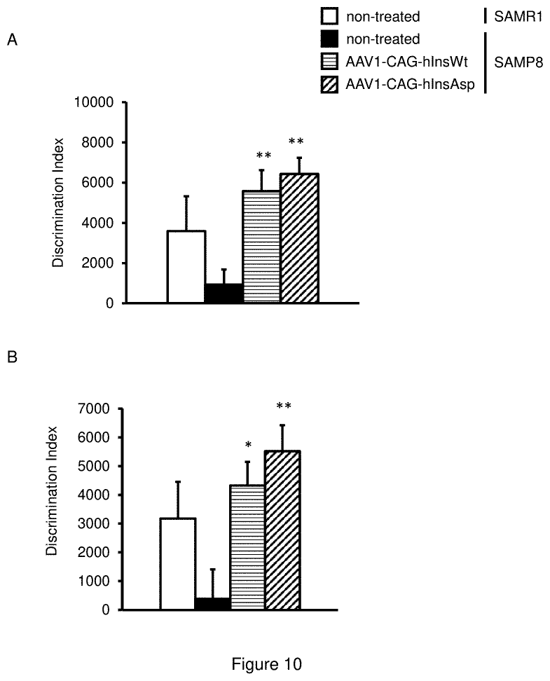

To test the effect of the intra-CSF treatment with viral vectors encoding Insulin on memory, the novel object recognition test was performed at 33 weeks of age. SAMP8 mice treated with either AAV1-CAG-hlnsAsp or AAV1-CAG-hlnsWt-encoding vectors performed markedly better than the untreated SAMP8 cohort (FIG. 10), and their discrimination index was similar than that of the SAMR1 non-treated control mice, both 10 minutes (FIG. 10A) and 24 hours (FIG. 10B) after the first trial, indicating increased short and long term memory after the gene therapy.

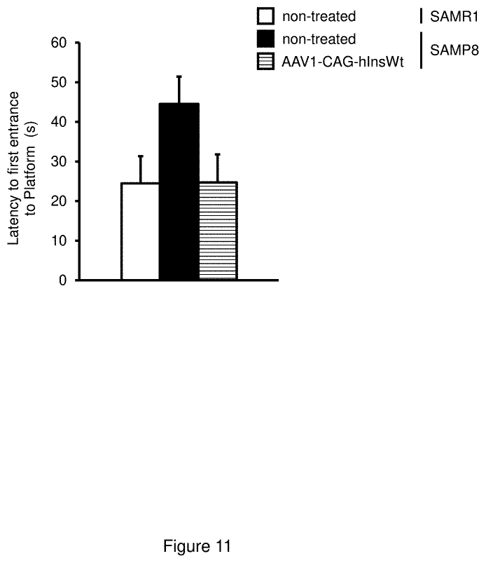

The learning capacity was evaluated in AAV1-CAG-hlnsWt mice at 39 weeks of age with the Morris Water Maze test and the latency to first entrance the platform of treated mice was reduced in SAMP8 mice after gene therapy treatment (FIG. 11), indicating that AAV1-CAG-hlnsWt administration into the CNS enhances learning capacity in SAMP8 mice.

Sequences

| SEQ | |
|---|---|
| ID | |
| NO: | Description of the sequence |
| 1 | Amino acid sequence of |
| | Homo sapiens insulin |
| 2 | Amino acid sequence of |
| | Mus musculus insulin |
| 3 | Amino acid sequence of |
| | Canis lupus familiaris |
| | insulin |
| 4 | Nucleotide sequence of |
| | Homo sapiens insulin |
| 5 | Nucleotide sequence of |
| | Mus musculus insulin |
| 6 | Nucleotide sequence of |
| | Canis lupus familiaris |
| | insulin |
| 7 | Nucleotide sequence encoding |
| | miRT-122a |
| 8 | Nucleotide sequence encoding |
| | miRT-1 |
| 9 | Nucleotide sequence encoding |
| | miRT-152 |
| 10 | Nucleotide sequence encoding |
| | miRT-199a-5p |
| 11 | Nucleotide sequence encoding |
| | miRT-199a-3p |
| 12 | Nucleotide sequence encoding |
| | miRT-215 |
| 13 | Nucleotide sequence encoding |
| | miRT-192 |
| 14 | Nucleotide sequence encoding |
| | miRT-148a |
| 15 | Nucleotide sequence encoding |
| | miRT-194 |
| 16 | Nucleotide sequence encoding |
| | miRT-133a |
| 17 | Nucleotide sequence encoding |
| | miRT-206 |
| 18 | Nucleotide sequence encoding |
| | miRT-208a-5p |
| 19 | Nucleotide sequence encoding |
| | miRT-208a-3p |
| 20 | Nucleotide sequence encoding |
| | miRT-499-5p |
| 21 | Nucleotide sequence of |
| | chimeric intron composed |
| | of introns from human |
| | β-globin and immunoglobulin |
| | heavy chain genes |
| 22 | Nucleotide sequence of |
| | CAG promoter |
| 23 | Nucleotide sequence of |
| | CMV promoter |
| 24 | Nucleotide sequence of |
| | CMV enhancer |
| 25 | mini-CMV promoter |
| 26 | EF1α promoter |
| 27 | RSV promoter |
| 28 | Synapsin 1 promoter |
| 29 | Calcium/calmodulin-dependent |
| | protein kinase II |
| | (CaMKII) promoter |
| 30 | Glial fibrillary acidic |
| | protein (GFAP) promoter |
| 31 | Nestin promoter |
| 32 | Homeobox Protein 9 (HB9) |
| | promoter |
| 33 | Tyrosine hydroxylase (TH) |
| | promoter |
| 34 | Myelin basic protein (MBP) |
| | promoter |
| 35 | Truncated AAV2 5′ ITR |
| 36 | Truncated AAV2 3′ ITR |
| 37 | SV40 polyadenylation signal |
| 38 | Rabbit β-globin polyadenylation |
| | signal |
| 39 | CMV promoter and CMV enhancer |
| | sequence |
| 40 | pAAV-CAG-hIns-d miRT |
| 41 | Amino acid sequence of |
| | H. sapiens insulin with |
| | furin cleavage sites |
| 42 | Amino acid sequence of |
| | H. sapiens insulin mutant |
| | His-B10-Asp with furin |
| | cleavage sites |
| 43 | Amino acid sequence of |
| | R. norvegicus insulin |
| 44 | Amino acid sequence of |
| | P. troglodytes insulin |
| 45 | Nucleotide sequence of |
| | H. sapiens insulin with |
| | furin cleavage sites |
| 46 | Nucleotide sequence of |
| | H. sapiens insulin mutant |
| | His-B10-Asp with furin |
| | cleavage sites |
| 47 | Nucleotide sequence of |
| | R. norvegicus insulin |
| 48 | Nucleotide sequence of |
| | P. troglodytes insulin |
| 49 | pAAV-CAG-hInsAsp |
| 50 | pAAV-CAG-hInsWt |

```
Nucleotide sequence H. sapiens insulin
(SEQ ID NO: 4)
ATGGCCCTGTGGATGCGCCTCCTGCCCCTGCTGGCGCTGC
TGGCCCTCTGGGGACCTGACCCAGCCGCAGCCTTTGTGAA
CCAACACCTGTGCGGCTCACACCTGGTGGAAGCTCTCTAC
CTAGTGTGCGGGGAACGAGGCTTCTTCTACACACCCAAGA
CCCGCCGGGAGGCAGAGGACCTGCAGGTGGGGCAGGTGGA
GCTGGGCGGGGGCCCTGGTGCAGGCAGCCTGCAGCCCTTG
GCCCTGGAGGGGTCCCTGCAGAAGCGTGGCATTGTGGAAC
AATGCTGTACCAGCATCTGCTCCCTCTACCAGCTGGAGAA
CTACTGCAACTAG
Amino acid sequence H. sapiens insulin
(SEQ ID NO: 1)
MALWMRLLPLLALLALWGPDPAAAFVNQHLCGSHLVEALY
LVCGERGFFYTPKTRREAEDLQVGQVELGGGPGAGSLQPL
ALEGSLQKRGIVEQCCTSICSLYQLENYCN
Nucleotide sequence H. sapiens insulin with
furin cleaving sites
(SEQ ID NO: 45)
ATGGCCCTGTGGATGCGCCTCCTGCCCCTGCTGGCGCTGC
TGGCCCTCTGGGGACCTGACCCAGCCGCAGCCTTTGTGAA
CCAACACCTGTGCGGCTCACACCTGGTGGAAGCTCTCTAC
CTAGTGTGCGGGGAACGAGGCTTCTTCTACACACCCAGGA
CCAAGCGGGAGGCAGAGGACCTGCAGGTGGGGCAGGTGGA
GCTGGGCGGGGGCCCTGGTGCAGGCAGCCTGCAGCCCTTG
GCCCTGGAGGGGTCGCGACAGAAGCGTGGCATTGTGGAAC
AATGCTGTACCAGCATCTGCTCCCTCTACCAGCTGGAGAA
CTACTGCAACTAG
Amino acid sequence H. sapiens insulin with
furin cleaving sites
(SEQ ID NO: 41)
MALWMRLLPLLALLALWGPDPAAAFVNQHLCGSHLVEALY
LVCGERGFFYTPRTKREAEDLQVGQVELGGGPGAGSLQPL
ALEGSRQKRGIVEQCCTSICSLYQLENYCN
Nucleotide sequence H. sapiens insulin mutant
(His- B10-Asp) with furin cleaving sites
(SEQ ID NO: 46)
ATGGCCCTGTGGATGCGCCTCCTGCCCCTGCTGGCGCTGC
TGGCCCTCTGGGGACCTGACCCAGCCGCAGCCTTTGTGAA
CCAACACCTGTGCGGCTCAGATCTGGTGGAAGCTCTCTAC
CTAGTGTGCGGGGAACGAGGCTTCTTCTACACACCCAGGA
CCAAGCGGGAGGCAGAGGACCTGCAGGTGGGGCAGGTGGA
GCTGGGCGGGGGCCCTGGTGCAGGCAGCCTGCAGCCCTTG
GCCCTGGAGGGGTCGCGACAGAAGCGTGGCATTGTGGAAC
AATGCTGTACCAGCATCTGCTCCCTCTACCAGCTGGAGAA
CTACTGCAACTAG
Amino acid sequence H. sapiens insulin mutant
(His-B10-Asp) with furin cleaving sites
(SEQ ID NO: 42)
MALWMRLLPLLALLALWGPDPAAAFVNQHLCGSDLVEALY
LVCGERGFFYTPRTKREAEDLQVGQVELGGGPGAGSLQPL
ALEGSRQKRGIVEQCCTSICSLYQLENYCN
Nucleotide sequence M. musculus insulin
(SEQ ID NO: 5)
ATGGCCCTGTGGATGCGCTTCCTGCCCCTGCTGGCCCTGC
TCTTCCTCTGGGAGTCCCACCCCACCCAGGCTTTTGTCAA
GCAGCACCTTTGTGGTTCCCACCTGGTGGAGGCTCTCTAC
CTGGTGTGTGGGGAGCGTGGCTTCTTCTACACACCCATGT
CCCGCCGTGAAGTGGAGGACCCACAAGTGGCACAACTGGA
GCTGGGTGGAGGCCCGGGAGCAGGTGACCTTCAGACCTTG
GCACTGGAGGTGGCCCAGCAGAAGCGTGGCATTGTAGATC
AGTGCTGCACCAGCATCTGCTCCCTCTACCAGCTGGAGAA
CTACTGCAACTAG
Amino acid sequence M. musculus insulin
(SEQ ID NO: 2)
MALWMRFLPLLALLFLWESHPTQAFVKQHLCGSHLVEALY
LVCGERGFFYTPMSRREVEDPQVAQLELGGGPGAGDLQTL
ALEVAQQKRGIVDQCCTSICSLYQLENYCN
Nucleotide sequence R. norvegicus insulin
(SEQ ID NO: 47)
atggccctgtggatccgcttcctgcccctgctggccctgc
tcatcctctgggagccccgccctgcccaggcttttgtcaa
acagcacctttgtggttctcacttggtggaagctctctac
ctggtgtgtggggagcgtggattcttctacacacccatgt
cccgccgcgaagtggaggacccacaagtggcacaactgga
gctgggtggaggcccgggggcaggtgaccttcagaccttg
gcactggaggtggcccggcagaagcgcggcatcgtggatc
agtgctgcaccagcatctgctctctctaccaactggagaa
ctactgcaactag
Amino acid sequence R. norvegicus insulin
(SEQ ID NO: 43)
MALWIRFLPLLALLILWEPRPAQAFVKQHLCGSHLVEALY
LVCGERGFFYTPMSRREVEDPQVAQLELGGGPGAGDLQTL
ALEVARQKRGIVDQCCTSICSLYQLENYCN
Nucleotide sequence C. lupus familiaris insulin
(SEQ ID NO: 61
atggccctctggatgcgcctcctgcccctgctggccctgc
tggccctctgggcgcccgcgcccacccgagccttcgttaa
ccagcacctgtgtggctcccacctggtagaggctctgtac
ctggtgtgcggggagcgcggcttcttctacacgcctaagg
cccgccgggaggtggaggacctgcaggtgagggacgtgga
gctggccggggcgcctggcgagggcggcctgcagcccctg
gccctggagggggccctgcagaagcgaggcatcgtggagc
agtgctgcaccagcatctgctccctctaccagctggagaa
ttactgcaactag
Amino acid sequence C. lupus familiaris insulin
(SEQ ID NO: 3)
MALWMRLLPLLALLALWAPAPTRAFVNQHLCGSHLVEALY
LVCGERGFFYTPKARREVEDLQVRDVELAGAPGEGGLQPL
ALEGALQKRGIVEQCCTSICSLYQLENYCN
Nucleotide sequence P. troglodytes insulin
(SEQ ID NO: 48)
atggccctgtggatgcgcctcctgcccctgctggtgctgc
tggccctctggggacctgacccagcctcggcctttgtgaa
ccaacacctgtgcggctcccacctggtggaagctctctac
ctagtgtgcggggaacgaggcttcttctacacacccaaga
cccgccgggaggcagaggacctgcaggtggggcaggtgga
gctgggcgggggccctggtgcaggcagcctgcagcccttg
gccctggaggggtccctgcagaagcgtggtatcgtggaac
aatgctgtaccagcatctgctccctctaccagctggagaa
ctactgcaactag
Amino acid sequence P. troglodytes insulin
(SEQ ID NO: 44)
MALWMRLLPLLVLLALWGPDPASAFVNQHLCGSHLVEALY
LVCGERGFFYTPKTRREAEDLQVGQVELGGGPGAGSLQPL
ALEGSLQKRGIVEQCCTSICSLYQLENYCN
Nucleotide sequence of CAG promoter
(SEQ ID NO: 22)
gacattgattattgactagttattaatagtaatcaattac
ggggtcattagttcatagcccatatatggagttccgcgtt
acataacttacggtaaatggcccgcctggctgaccgccca
acgacccccgcccattgacgtcaataatgacgtatgttcc
catagtaacgccaatagggactttccattgacgtcaatgg
gtggagtatttacggtaaactgcccacttggcagtacatc
aagtgtatcatatgccaagtacgccccctattgacgtcaa
tgacggtaaatggcccgcctggcattatgcccagtacatg
accttatgggactttcctacttggcagtacatctacgtat
tagtcatcgctattaccatggtcgaggtgagccccacgtt
ctgcttcactctccccatctcccccccctccccaccccca
attttgtatttatttattt
Nucleotide sequence M. musculus insulin
(SEQ ID NO: 5)
ATGGCCCTGTGGATGCGCTTCCTGCCCCTGCTGGCCCTGC
TCTTCCTCTGGGAGTCCCACCCCACCCAGGCTTTTGTCAA
GCAGCACCTTTGTGGTTCCCACCTGGTGGAGGCTCTCTAC
CTGGTGTGTGGGGAGCGTGGCTTCTTCTACACACCCATGT
CCCGCCGTGAAGTGGAGGACCCACAAGTGGCACAACTGGA
GCTGGGTGGAGGCCCGGGAGCAGGTGACCTTCAGACCTTG
GCACTGGAGGTGGCCCAGCAGAAGCGTGGCATTGTAGATC
AGTGCTGCACCAGCATCTGCTCCCTCTACCAGCTGGAGAA
CTACTGCAACTAG
Amino acid sequence M. musculus insulin
(SEQ ID NO: 2)
MALWMRFLPLLALLFLWESHPTQAFVKQHLCGSHLVEALY
LVCGERGFFYTPMSRREVEDPQVAQLELGGGPGAGDLQTL
ALEVAQQKRGIVDQCCTSICSLYQLENYCN
Nucleotide sequence R. norvegicus insulin
(SEQ ID NO: 47)
atggccctgtggatccgcttcctgcccctgctggccctgc
tcatcctctgggagccccgccctgcccaggcttttgtcaa
acagcacctttgtggttctcacttggtggaagctctctac
ctggtgtgtggggagcgtggattcttctacacacccatgt
cccgccgcgaagtggaggacccacaagtggcacaactgga
gctgggtggaggcccgggggcaggtgaccttcagaccttg
gcactggaggtggcccggcagaagcgcggcatcgtggatc
agtgctgcaccagcatctgctctctctaccaactggagaa
ctactgcaactag
Amino acid sequence R. norvegicus insulin
(SEQ ID NO: 43)
MALWIRFLPLLALLILWEPRPAQAFVKQHLCGSHLVEALY
LVCGERGFFYTPMSRREVEDPQVAQLELGGGPGAGDLQTL
ALEVARQKRGIVDQCCTSICSLYQLENYCN
Nucleotide sequence C. lupus familiaris insulin
(SEQ ID NO: 61
atggccctctggatgcgcctcctgcccctgctggccctgc
tggccctctgggcgcccgcgcccacccgagccttcgttaa
ccagcacctgtgtggctcccacctggtagaggctctgtac
ctggtgtgcggggagcgcggcttcttctacacgcctaagg
cccgccgggaggtggaggacctgcaggtgagggacgtgga
gctggccggggcgcctggcgagggcggcctgcagcccctg
gccctggagggggccctgcagaagcgaggcatcgtggagc
agtgctgcaccagcatctgctccctctaccagctggagaa
ttactgcaactag
Amino acid sequence C. lupus familiaris insulin
(SEQ ID NO: 3)
MALWMRLLPLLALLALWAPAPTRAFVNQHLCGSHLVEALY
LVCGERGFFYTPKARREVEDLQVRDVELAGAPGEGGLQPL
ALEGALQKRGIVEQCCTSICSLYQLENYCN
Nucleotide sequence P. troglodytes insulin
(SEQ ID NO: 48)
atggccctgtggatgcgcctcctgcccctgctggtgctgc
tggccctctggggacctgacccagcctcggcctttgtgaa
ccaacacctgtgcggctcccacctggtggaagctctctac
ctagtgtgcggggaacgaggcttcttctacacacccaaga
cccgccgggaggcagaggacctgcaggtggggcaggtgga
gctgggcgggggccctggtgcaggcagcctgcagcccttg
gccctggaggggtccctgcagaagcgtggtatcgtggaac
aatgctgtaccagcatctgctccctctaccagctggagaa
ctactgcaactag
Amino acid sequence P. troglodytes insulin
(SEQ ID NO: 44)
MALWMRLLPLLVLLALWGPDPASAFVNQHLCGSHLVEALY
LVCGERGFFYTPKTRREAEDLQVGQVELGGGPGAGSLQPL
ALEGSLQKRGIVEQCCTSICSLYQLENYCN
Nucleotide sequence of CAG promoter
(SEQ ID NO: 22)
gacattgattattgactagttattaatagtaatcaattac
ggggtcattagttcatagcccatatatggagttccgcgtt
acataacttacggtaaatggcccgcctggctgaccgccca
acgacccccgcccattgacgtcaataatgacgtatgttcc
catagtaacgccaatagggactttccattgacgtcaatgg
gtggagtatttacggtaaactgcccacttggcagtacatc
aagtgtatcatatgccaagtacgccccctattgacgtcaa
tgacggtaaatggcccgcctggcattatgcccagtacatg
accttatgggactttcctacttggcagtacatctacgtat
tagtcatcgctattaccatggtcgaggtgagccccacgtt
ctgcttcactctccccatctcccccccctccccaccccca
attttgtatttatttattttttaattattttgtgcagcga
tgggggcggggggggggggggggcgcgcgccaggcggggc
ggggcggggcgaggggcggggcggggcgaggcggagaggt
gcggcggcagccaatcagagcggcgcgctccgaaagtttc
cttttatggcgaggcggcggcggcggcggccctataaaaa
gcgaagcgcgcggcgggcgggagtcgctgcgttgccttcg
ccccgtgccccgctccgcgccgcctcgcgccgcccgcccc
ggctctgactgaccgcgttactcccacaggtgagcgggcg
ggacggcccttctcctccgggctgtaattagcgcttggtt
taatgacggcttgtttcttttctgtggctgcgtgaaagcc
ttgaggggctccgggagggccctttgtgcggggggagcgg
ctcggggggtgcgtgcgtgtgtgtgtgcgtggggagcgcc
gcgtgcggctccgcgctgcccggcggctgtgagcgctgcg
ggcgcggcgcggggctttgtgcgctccgcagtgtgcgcga
ggggagcgcggccgggggcggtgccccgcggtgcgggggg
ctgcgaggggaacaaaggctgcgtgcggggtgtgtgcgtg
ggggggtgagcagggggtgtgggcgcgtcggtcgggctgc
aaccccccctgcacccccctccccgagttgctgagcacgg
cccggcttcgggtgcggggctccgtacggggcgtggcgcg
gggctcgccgtgccgggcggggggtggcggcaggtggggg
tgccgggcggggcggggccgcctcgggccggggagggctc
gggggaggggcgcggcggcccccggagcgccggcggctgt
cgaggcgcggcgagccgcagccattgccttttatggtaat
cgtgcgagagggcgcagggacttcctttgtcccaaatctg
tgcggagccgaaatctgggaggcgccgccgcaccccctct
agcgggcgcggggcgaagcggtgcggcgccggcaggaagg
aaatgggcggggagggccttcgtgcgtcgccgcgccgccg
tccccttctccctctccagcctcggggctgtccgcggggg
gacggctgccttcgggggggacggggcagggcggggttcg
gcttctggcgtgtgaccggcggctctagagcctctgctaa
ccatgttcatgccttcttctttttcctacag
Nucleotide sequence of CMV promoter
(SEQ ID NO: 23)
gtgatgcggttttggcagtacaccaatgggcgtggatagc
ggtttgactcacggggatttccaagtctccaccccattga
cgtcaatgggagtttgttttggcaccaaaatcaacgggac
tttccaaaatgtcgtaacaactgcgatcgcccgccccgtt
gacgcaaatgggcggtaggcgtgtacggtgggaggtctat
ataagcagagct
Nucleotide sequence of CMV enhancer
(SEQ ID NO: 241
ggcattgattattgactagttattaatagtaatcaattac
ggggtcattagttcatagcccatatatggagttccgcgtt
acataacttacggtaaatggcccgcctggctgaccgccca
acgacccccgcccattgacgtcaataatgacgtatgttcc
catagtaacgccaatagggactttccattgacgtcaatgg
gtggagtatttacggtaaactgcccacttggcagtacatc
aagtgtatcatatgccaagtccgccccctattgacgtcaa
tgacggtaaatggcccgcctggcattatgcccagtacatg
accttacgggactttcctacttggcagtacatctacgtat
tagtcatcgctattaccatg
CMV promoter and CMV enhancer sequence
(SEQ ID NO: 39)
ggcattgattattgactagttattaatagtaatcaattac
ggggtcattagttcatagcccatatatggagttccgcgtt
acataacttacggtaaatggcccgcctggctgaccgccca
acgacccccgcccattgacgtcaataatgacgtatgttcc
catagtaacgccaatagggactttccattgacgtcaatgg
gtggagtatttacggtaaactgcccacttggcagtacatc
aagtgtatcatatgccaagtccgccccctattgacgtcaa
tgacggtaaatggcccgcctggcattatgcccagtacatg
accttacgggactttcctacttggcagtacatctacgtat
tagtcatcgctattaccatggtgatgcggttttggcagta
caccaatgggcgtggatagcggtttgactcacggggattt
ccaagtctccaccccattgacgtcaatgggagtttgtttt
ggcaccaaaatcaacgggactttccaaaatgtcgtaacaa
ctgcgatcgcccgccccgttgacgcaaatgggcggtaggc
gtgtacggtgggaggtctatataagcagagct
truncated AAV2 5′ ITR
(SEQ ID NO: 35)
gcgcgctc gctcgctcac tgaggccgcc cgggcaaagc
ccgggcgtcg ggcgaccttt ggtcgcccgg cctcagtgag
cgagcgagcg cgcagagagg gagtggccaa ctccatcact
aggggttcct
truncated AAV2 3′ ITR
(SEQ ID NO: 36)
aggaacccct agtgatggag ttggccactc cctctctgcg
cgctcgctcg ctcactgagg ccgggcgacc aaaggtcgcc
cgacgcccgg gctttgcccg ggcggcctca gtgagcgagc
gagcgcgc
Rabbit β-globin polyadenylation signal
(3′ UTR and flanking region of
rabbit beta-globin, including
polvA signal)
(SEQ ID NO: 38)
gatctttttccctctgccaaaaattatggggacatcatga
agccccttgagcatctgacttctggctaataaaggaaatt
tattttcattgcaatagtgtgttggaattttttgtgtctc
tcactcggaaggacatatgggagggcaaatcatttaaaac
atcagaatgagtatttggtttagagtttggcaacatatgc
ccatatgctggctgccatgaacaaaggttggctataaaga
ggtcatcagtatatgaaacagccccctgctgtccattcct
tattccatagaaaagccttgacttgaggttagattttttt
tatattttgttttgtgttatttttttctttaacatcccta
aaattttccttacatgttttactagccagatttttcctcc
tctcctgactactcccagtcatagctgtccctcttctctt
atggagatc
miRT sequences
miRT-122a (SEQ ID NO: 7):
5′ CAAACACCATTGTCACACTCCA 3′, target for the
microRNA-122a (Accession Number to the miRBase
database MI0000442), which is expressed in the
liver.
miRT-152 (SEQ ID NO: 9):
5′ CCAAGTTCTGTCATGCACTGA 3′, target for the
microRNA-152 (MI0000462), which is expressed
in the liver.
miRT-199a-5p (SEQ ID NO: 10):
5′ GAACAGGTAGTCTGAACACTGGG 3′ target for the
microRNA 199a (MI0000242), which is expressed in
the liver.
miRT-199a-3p (SEQ ID NO: 11):
5′ TAACCAATGTGCAGACTACTGT 3′, target for the
microRNA-199a (MI0000242), which is expressed in
the liver.
miRT-215 (SEQ ID NO: 12):
5′ GTCTGTCAATTCATAGGTCAT 3′, target for the
microRNA-215 (MI0000291), which is expressed in
the liver.
miRT-192 (SEQ ID NO: 13):
5′ GGCTGTCAATTCATAGGTCAG 3′, target for the
microRNA-192 (MI0000234), which is expressed in
the liver.
miRT-148a (SEQ ID NO: 14):
5′ ACAAAGTTCTGTAGTGCACTGA 3′ target for the
microRNA-148a (MI0000253), which is expressed in
the liver.
miRT-194 (SEQ ID NO: 15):
5′ TCCACATGGAGTTGCTGTTACA 3′, target for the
microRNA-194 (MI0000488), which is expressed in
the liver.
miRT-133a (SEQ ID NO: 16):
5′ CAGCTGGTTGAAGGGGACCAAA 3′, target for the
microRNA-133a (MI0000450), which is expressed
in the heart.
miRT-206 (SEQ ID NO: 17):
5′ CCACACACTTCCTTACATTCCA 3′, target for the
microRNA-206 (MI0000490), which is expressed
in the heart.
miRT-1 (SEQ ID NO: 8):
5′ TTACATACTTCTTTACATTCCA 3′, target for the
microRNA-1 (MI0000651), which is expressed
in the heart.
miRT-208a-5p (SEQ ID NO: 18):
5′ GTATAACCCGGGCCAAAAGCTC 3′, target for the
microRNA-208a (MI0000251), which is expressed
in the heart.
miRT-208a-3p (SEQ ID NO: 19):
5′ ACAAGCTTTTTGCTCGTCTTAT 3′, target for the
microRNA- 208a (MI0000251), which is expressed
in the heart.
miRT-499-5p (SEQ ID NO: 20):
5′ AAACATCACTGCAAGTCTTAA 3′, target for the
microRNA- 499 (MI0003183), which is expressed
in the heart.
Mini-CMV: cmv intermediate early promoter
(SEQ ID NO: 25)
tatgccaagtacgccccctattgacgtcaatgacggtaaa
tggcccgcctggcattatgcccagtacatgaccttatggg
actttcctacttggcagtacatctacgtattagtcatcgc
tattaccatggtgatgcggttttggcagtacatcaatggg
cgtggatagcggtttgactcacggggatttccaagtctcc
accccattgacgtcaatgggagtttgttttggcaccaaaa
tcaacgggactttccaaaatgtcgtaacaactccgcccca
ttgacgcaaatgggcggtaggcgtgtacggtgggaggtct
atataagcagagctctctggctaactagagaacccactgc
ttaactggcttatcgaaattaatacgactcactataggga
gacccaagctt
Nucleotide sequence of EF1α promoter
(SEQ ID NO: 26)
ggctccggtgcccgtcagtgggcagagcgcacatcgccca
cagtccccgagaagttggggggaggggtcggcaattgaac
cggtgcctagagaaggtggcgcggggtaaactgggaaagt
gatgtcgtgtactggctccgcctttttcccgagggtgggg
gagaaccgtatataagtgcagtagtcgccgtgaacgttct
ttttcgcaacgggtttgccgccagaacacaggtaagtgcc
gtgtgtggttcccgcgggcctggcctctttacgggttatg
gcccttgcgtgccttgaattacttccactggctgcagtac
gtgattcttgatcccgagcttcgggttggaagtgggtggg
agagttcgaggccttgcgcttaaggagccccttcgcctcg
tgcttgagttgaggcctggcctgggcgctggggccgccgc
gtgcgaatctggtggcaccttcgcgcctgtctcgctgctt
tcgataagtctctagccatttaaaatttttgatgacctgc
tgcgacgctttttttctggcaagatagtcttgtaaatgcg
ggccaagatctgcacactggtatttcggtttttggggccg
cgggcggcgacggggcccgtgcgtcccagcgcacatgttc
ggcgaggcggggcctgcgagcgcggccaccgagaatcgga
cgggggtagtctcaagctggccggcctgctctggtgcctg
gcctcgcgccgccgtgtatcgccccgccctgggcggcaag
gctggcccggtcggcaccagttgcgtgagcggaaagatgg
ccgcttcccggccctgctgcagggagctcaaaatggagga
cgcggcgctcgggagagcgggcgggtgagtcacccacaca
aaggaaaagggcctttccgtcctcagccgtcgcttcatgt
gactccacggagtaccgggcgccgtccaggcacctcgatt
agttctcgagcttttggagtacgtcgtctttaggttgggg
ggaggggttttatgcgatggagtttccccacactgagtgg
gtggagactgaagttaggccagcttggcacttgatgtaat
tctccttggaatttgccctttttgagtttggatcttggtt
cattctcaagcctcagacagtggttcaaagtttttttctt
ccatttcaggtgtcgtga
Nucleotide sequence of RSV promoter
(SEQ ID NO: 27)
catgtttgacagcttatcatcgcagatcc
gtatggtgcactctcagtacaatctgctctgatgccgcat
agttaagccagtatctgctccctgcttgtgtgttggaggt
cgctgagtagtgcgcgagcaaaatttaagctacaacaagg
caaggcttgaccgacaattgcatgaagaatctgcttaggg
ttaggcgttttgcgctgcttcgcgatgtacgggccagata
ttcgcgtatctgaggggactagggtgtgtttaggcgaaaa
gcggggcttcggttgtacgcggttaggagtcccctcagga
tatagtagtttcgcttttgcatagggagggggaaatgtag
tcttatgcaatactcttgtagtcttgcaacatggtaacga
tgagttagcaacatgccttacaaggagagaaaaagcaccg
tgcatgccgattggtggaagtaaggtggtacgatcgtgcc
ttattaggaaggcaacagacgggtctgacatggattggac
gaaccactaaattccgcattgcagagatattgtatttaag
tgcctagctcgatacaataaacgccatttgaccattcacc
acattggtgtgcacctccaagctgggtaccagct
Synapsin 1 promoter (SEQ ID NO: 28)
ctgcgctctcaggcacgacacgactcctccgctgcccacc
gcagactgaggcagcgctgagtcgccggcgccgcagcgca
gatggtcgcgcccgtgcccccctatctcgcgcctcgcgtg
gtgcggtccggctgggccggcggcggcgcggacgcgacca
aggtggccgggaaggggagtttgcgggggaccggcgagtg
acgtcagcgcgccttcagtgctgaggcggcggtggcgcgc
gccgccaggcgggggcgaaggcactgtccgcggtgctgaa
gctggcagtgcgcacgcgcctcgccgcatcctgtttcccc
tccccctctctgataggggatgcgcaatttggggaatggg
ggttgggtgcttgtccagtgggtcggggtcggtcgtcagg
taggcacccccaccccgcctcatcctggtcctaaaaccca
cttgcact
Calcium/calmodulin-dependent protein
kinase II (CaMKII) promoter
(SEQ ID NO: 29)
taacattatggccttaggtcacttcatctccatggggttc
ttcttctgattttctagaaaatgagatgggggtgcagaga
gcttcctcagtgacctgcccagggtcacatcagaaatgtc
agagctagaacttgaactcagattactaatcttaaattcc
atgccttgggggcatgcaagtacgatatacagaaggagtg
aactcattagggcagatgaccaatgagtttaggaaagaag
agtccagggcagggtacatctacaccacccgcccagccct
gggtgagtccagccacgttcacctcattatagttgcctct
ctccagtcctaccttgacgggaagcacaagcagaaactgg
gacaggagccccaggagaccaaatcttcatggtccctctg
ggaggatgggtggggagagctgtggcagaggcctcaggag
gggccctgctgctcagtggtgacagataggggtgagaaag
cagacagagtcattccgtcagcattctgggtctgtttggt
acttcttctcacgctaaggtggcggtgtgatatgcacaat
ggctaaaaagcagggagagctggaaagaaacaaggacaga
gacagaggccaagtcaaccagaccaattcccagaggaagc
aaagaaaccattacagagactacaagggggaagggaagga
gagatgaattagcttcccctgtaaaccttagaacccagct
gttgccagggcaacggggcaatacctgtctcttcagagga
gatgaagttgccagggtaactacatcctgtctttctcaag
gaccatcccagaatgtggcacccactagccgttaccatag
caactgcctctttgccccacttaatcccatcccgtctgtt
aaaagggccctatagttggaggtgggggaggtaggaagag
cgatgatcacttgtggactaagtttgttcgcatccccttc
tccaaccccctcagtacatcaccctgggggaacagggtcc
acttgctcctgggcccacacagtcctgcagtattgtgtat
ataaggccagggcaaagaggagcaggttttaaagtgaaag
gcaggcaggtgttggggaggcagttaccggggcaacggga
acagggcgtttcggaggtggttgccatggggacctggatg
ctgacgaaggctcgcgaggctgtgagcagccacagtgccc
tgctcagaagccccaagctcgtcagtcaagccggttctcc
gtttgcactcaggagcacgggcaggcgagtggcccctagt
tctgggggcagcgggg
Glial fibrillary acidic protein
(GFAP) promoter (SEQ ID NO: 30)
cgcgtgatctaacatatcctggtgtggagtaggggacgct
gctctgacagaggctcgggggcctgagctggctctgtgag
ctggggaggaggcagacagccaggccttgtctgcaagcag
acctggcagcattgggctggccgccccccagggcctcctc
ttcatgcccagtgaatgactcaccttggcacagacacaat
gttcggggtgggcacagtgcctgcttcccgccgcacccca
gcccccctcaaatgccttccgagaagcccattgagcaggg
ggcttgcattgcaccccagcctgacagcctggcatcttgg
gataaaagcagcacagccccctaggggctgcccttgctgt
gtggcgccaccggcggtggagaacaaggctctattcagcc
tgtgcccaggaaaggggatcaggggatgcccaggcatgga
cagtgggtggcagggggggagaggagggctgtctgcttcc
cagaagtccaaggacacaaatgggtgaggggagagctctc
cccatagctgggctgcggcccaaccccaccccctcaggct
atgccagggggtgttgccaggggcacccgggcatcgccag
tctagcccactccttcataaagccctcgcatcccaggagc
gagcagagccagagcaggttggagaggagacgcatcacct
ccgctgctcgcggggtctagagtcga
Nestin promoter (SEQ ID NO: 31)
gaaggcagcccccggaggtcaaaggctgggcacgcgggag
gagaggccagagtcagaggctgcgggtatctcagatatga
aggaaagatgagagaggctcaggaagaggtaagaaaagac
acaagagaccagagaagggagaagaattagagagggaggc
agaggaccgctgtctctacagacatagctggtagagactg
ggaggaagggatgaaccctgagcgcatgaagggaaggagg
tggctggtggtatatggaggatgtagctgggccagggaaa
agatcctgcactaaaaatctgaagctaaaaataacaggac
acggggtggagaggcgaaaggagggcagattgaggcagag
agactgagaggcctggggatgtgggcattccggtagggca
cacagttcacttgtcttctctttttccaggaggccaaaga
tgctgacctcaagaactcataataccccagtggggaccac
cgcattcatagccctgttacaagaagtgggagatgttcct
ttttgtcccagactggaaatccattacatcccgaggctca
ggttctgtggtggtcatctctgtgtggcttgttctgtggg
cctacctaaagtcctaagcacagctctcaagcagatccga
ggcgactaagatgctagtaggggttgtctggagagaagag
ccgaggaggtgggctgtgatggatcagttcagctttcaaa
taaaaaggcgtttttatattctgtgtcgagttcgtgaacc
cctgtggtgggcttctccatctgtctgggttagtacctgc
cactatactggaataaggagacgcctgcttccctcgagtt
ggctggacaaggttatgagcatccgtgtacttatggggtt
gccagcttggtcctggatcgcccgggcccttcccccaccc
gttcggttccccaccaccacccgcgctcgtacgtgcgtct
ccgcctgcagctcttgactcatcggggcccccgggtcaca
tgcgctcgctcggctctataggcgccgccccctgcccacc
ccccgcccgcgctgggagccgcagccgccgccactcctgc
tctctctgcgccgccgccgtcaccaccgccaccgccaccg
gctgagtctgcagtcctccgaaacgggccctct
Homeobox Protein 9 promoter (HB9)
promoter (SEQ ID NO: 32)
tgaataaatttaagcaggctaattaatatataaactagct
caatttgtcaagttgatttgtattttagttaattgtgaaa
gtaattaccacatggtcaaattaacagctttctggaaatg
accaagcctgaggttttatttccttcctgggtgaagaaaa
ttcatttttccaagctcttgatgtgatgaataaaagtcat
aaatctgggtgattggtgcaggcagagtctaaatggcttc
atatttcattttaggtttaatagaaatattcatgctctgt
tttaatgaaattaaattgaagggggatggggctagagtgg
ttagctgatgaattgacaaaaactaatcagctttattggg
aaacaggtttaagggcacggacgtgtcaataacgctcagc
ctgaccccctcttccattagctaggcaggctgattaga
Tyrosine hydroxylase (TH1 promoter
(SEQ ID NO: 33)
ctgctaggggctgcttcccagctactcctcttggctccgt
ggcttgccttccagcctgtgtgctgtctggagagccttta
aagcctcacttccaccaactagaagtctctccccaaccct
gccctgacctcaagtgcacctcttcaaagtcaggtttagc
agctgcagctgggggccctgaatcccacccctgctgtctt
ccttgaagacagaagtgttgggagctgaggatctgggcta
gagactggctgtatgatccagagaagtagtgtgcttctgg
gcctcagatttcccttctgtagaacaggtttgtctgaaat
ggagaggttggtgctcctctgcagggcctagtgggagtca
ccatgagtggttaaaagatccagcttgtcttttggtgagc
tttgagaggaggtaacagggctgagttctggaagcctgac
caagggcagacttaaggggcctcttggagttgttctcatc
aaatggggatgggacacagctaaagtgcccagggcttctc
tgtgcccacagatgctttagatcttggcacagtgtggtct
accagctgtctctctctgtgtatatatatgtatttcatag
acagtgtacagtggcctggtttgtgctatcaggctggata
tggacagaggcaagagtttgtggcagcagttatctcccaa
gagagtccaaagacatcatgttttcaagtttaggccaggt
gctacttgagagagctcagacacagacaaaggtctggaga
gcacatgtcctccacccccacctagcttctgttgcaagca
cctccagccgagacaagagaacgaattaaaaagcaatatt
tgtgtcagtgtaagacatttgccgaaaggttaaatccaca
ttcgtgttgctgcagagcagccccctatgcaggatttgtt
agatacagctccgtcctaccctgtgccagctgagcaaacg
ccaggctgggtggggtggaacccagcctgggtttgcctca
ccctgcaatccccccagcaccctctaaaggaggaccctgt
ggtgggcatgcagacctagggactgggcatagataacctt
tgggtttgggcaacagcccccactcctcaggattgaaggc
taaggtgcagccagctctgccttcatggtgggaatgtctc
cacgtgacccctttctgggctgtggagaacactcagagaa
gagtcctgggatgccaggcaggccagggatgtgctgggca
tgttgagacaggagtgggctaagccagcagagttgctgac
ccaggaagagttcagaaaggggcatggaacatggggaggg
gtccatagtgagagagagcaggcagtgcagagtaaatagt
ccctgagctgggggttatgggatttgcaggagcttgctca
gagaaggcagaggagagatgctgcgccaagctgggtatca
cagagcctcagactcctggaacaggaactgtgggggtcag
gtcagcaggggaggttagggagtgttccctttgtactgac
ttagcatttatcctgcttctaggggggaaggggggccagt
gggggatgcacagcaaggcagtgatgtggcaggcagcctg
cgggagctcctggttcctggtgtgaaaaagctgggaagga
agagggctgggtctggtaagtacagcaggcagttggctcc
tgagagtccaagccctgtctagagggtggagtgagatttc
agagggagagctaaacggggtgggggctggggagtccagg
cttctggctcctgctaatactcagtgtgctgggtcctcag
aacctcagggtggccattttcagggtgagagctctgtcct
ttggcacttctgcagactccagtatccagaggaataaaga
tggtactcttcctcagttcccttagtgagaggacaccttt
ctctgaagggcttgggcagttgtcctgaaccattgcctga
aggaaggacttgactccagggacatagaatgggctcagca
taagtcccctgtagtagagaaaggtcccctctctggtctc
cttagagatcctgtttccttggctgaggaagctagggtgg
atctttgtgtaagtgggtgtggatgctcactggaaatcaa
aaggccccttggtgttagaccttggggtgccatgggagag
ttgatcactgagtgcgcccttacatgggggccagctgaga
atggggctgcctctagctcgagaccatgatgcagggagtg
agtgggggagttcaggatactcttaactaaagcagaggtc
tgtccccccagggaggggaggtcagaagaccctagggaga
tgccaaaggctagggttggcaccatgttgcaggctgtgtc
ttcaaggagatgataatcagaggaatcgaacctgcaaaag
tgggccagtcttagatacactatagaggaataatcttctg
aaacattctgtgtctcataggacctgcctgaggacccagc
cccagtgccagcacatacactggggcagtgagtagatagt
atactttgttacatgggctggggggacatggcctgtgccc
tggaggggacttgaagacatccaaaaagctagtgagaggg
ctcctagatttatttgtctccaagggctatatatagcctt
cctaacatgaacccttgggtaatccagcatgggcgctccc
atatgccctggtttgattagagagctctagatgtctcctg
tcccagaacaccagccagcccctgtcttcatgtcgtgtct
agggcggagggtgattcagaggcaggtgcctgcgacagtg
gatgcaattagatctaatgggacggaggcctctctcgtcc
gtcgccctcgctctgtgcccacccccgcctccctcaggca
cagcaggcgtggagaggatgcgcaggaggtaggaggtggg
ggacccagaggggctttgacgtcagcctggcctttaagag
gccgcctgcctggcaagggccgtggagacagaactcggga
ccaccagcttgcact
Myelin basic protein (MBP) promoter
(SEQ ID NO: 34)
caccgtggctttaacacttagagaaaatgcatcccctcta
atcaataagtcatcgacagtgggtagatggaggaacggca
gtgcgtagtaggatgcgtgcaagcatagtctcgtgcatgg
gtgcatagatcgctgggcaggtggacaaggtgggggtgga
taaagaagtgggtagatgattgatgttaggtaaatatcac
tgggtggacagatgggtggtaggtggatggatggttagaa
tagtcagaagagggatggattgataaggtgaacagatgat
aaatgggtgatagactggaagggttgtcaaaagaggataa
gggaagtgtgagctagccgtatttctaaggtcagtaatag
agttgggagaagaggttaagttacatccatttaaacctca
cacgaagctgagagggaatggacttgctgccgttggtgag
gaaagcgttgcatttcccgtgtgcttggttgtgaagtgct
caggtcccacatgaagcagtcaggttactgcggcttacag
aggagccagatccaaatgccccgagtaagcacgtccccga
gccagaggcctccagcggaatccgggagagggattgctca
gtgccctgcttccctggactgtaagctgcagaaagatgtg
ggaagtcctgttctccactgagaacactaaaagcaccttt
tgtcaaacgaccgcttcacatctggggcttgtgcactggt
ggccttttaaaccagagacaacccacaagatacctaacct
gcggggctctctggtacagtgagcaactcaggaaatgctt
tggcttgattgctgtgggctctcaggccatcgccctctgg
agtggttcttttaatgagaacctgaagattggcccctgag
ccatgtataccaagcaagctcaatccaggttagctccctc
tggttggggcaagctaacgtgctccttgggccccgcgcgt
aactgtgcgttttataggagacagctagttcaagacccca
ggaagaaagcggctttgtccccctctaggcctcgtacagg
cccacattcatatctcattgttgttgcaggggaggcagat
gcgatccagaacaatgggacctcggctgaggacacggcgg
tgacagactccaagcacacagcagacccaaagaataactg
gcaaggcgcccacccagctgacccagggaaccgcccccac
ttgatccgcctcttttcccgagatgccccgggaagggagg
acaacaccttcaaagacaggccctcagagtccgacgagct
tcagaccatccaagaagatcccacagcagcttccgaagaa
ttctgcagtcgacggtaccgcgggcccgggatc
SV40 polyA signal (SEQ ID NO: 37)
taagatacat tgatgagttt ggacaaacca caactagaat
gcagtgaaaatttgtgaaat ttgtgatgct attgctttat
ttgtaaccat tataagctgc aataaacaagtt
chimeric intron composed of introns from
human β-globin and immunoglobulin heavy
chain genes (SEQ ID NO: 21)
gtaagtatca aggttacaag acaggtttaa ggagaccaat
agaaactggg cttgtcgagacagagaagac tcttgcgttt
ctgataggca cctattggtc ttactgacat
ccactttgcctttctctcca cag
pAAV-CAG-hIns-dmiRT (SEQ ID NO: 40)
      1 AGTGAGCGAG CGAGCGCGCA GCTGCATTAA
          TGAATCGGCC AACGCGCGGG
    51 GAGAGGCGGT TTGCGTATTG GGCGCTCTTC
          CGCTTCCTCG CTCACTGACT
  101 CGCTGCGCTC GGTCGTTCGG CTGCGGCGAG
          CGGTATCAGC TCACTCAAAG
  151 GCGGTAATAC GGTTATCCAC AGAATCAGGG
          GATAACGCAG GAAAGAACAT
  201 GTGAGCAAAA GGCCAGCAAA AGGCCAGGAA
          CCGTAAAAAG GCCGCGTTGC
  251 TGGCGTTTTT CCATAGGCTC CGCCCCCCTG
          ACGAGCATCA CAAAAATCGA
  301 CGCTCAAGTC AGAGGTGGCG AAACCCGACA
          GGACTATAAA GATACCAGGC
  351 GTTTCCCCCT GGAAGCTCCC TCGTGCGCTC
          TCCTGTTCCG ACCCTGCCGC
  401 TTACCGGATA CCTGTCCGCC TTTCTCCCTT
          CGGGAAGCGT GGCGCTTTCT
  451 CATAGCTCAC GCTGTAGGTA TCTCAGTTCG
          GTGTAGGTCG TTCGCTCCAA
  501 GCTGGGCTGT GTGCACGAAC CCCCCGTTCA
          GCCCGACCGC TGCGCCTTAT
  551 CCGGTAACTA TCGTCTTGAG TCCAACCCGG
          TAAGACACGA CTTATCGCCA
  601 CTGGCAGCAG CCACTGGTAA CAGGATTAGC
          AGAGCGAGGT ATGTAGGCGG
  651 TGCTACAGAG TTCTTGAAGT GGTGGCCTAA
          CTACGGCTAC ACTAGAAGAA
  701 CAGTATTTGG TATCTGCGCT CTGCTGAAGC
          CAGTTACCTT CGGAAAAAGA
  751 GTTGGTAGCT CTTGATCCGG CAAACAAACC
          ACCGCTGGTA GCGGTGGTTT
  801 TTTTGTTTGC AAGCAGCAGA TTACGCGCAG
          AAAAAAAGGA TCTCAAGAAG
  851 ATCCTTTGAT CTTTTCTACG GGGTCTGACG
          CTCAGTGGAA CGAAAACTCA
  901 CGTTAAGGGA TTTTGGTCAT GAGATTATCA
          AAAAGGATCT TCACCTAGAT
  951 CCTTTTAAAT TAAAAATGAA GTTTTAAATC
          AATCTAAAGT ATATATGAGT
1001 AAACTTGGTC TGACAGTTAC CAATGCTTAA
          TCAGTGAGGC ACCTATCTCA
1051 GCGATCTGTC TATTTCGTTC ATCCATAGTT
          GCCTGACTCC CCGTCGTGTA
1101 GATAACTACG ATACGGGAGG GCTTACCATC
          TGGCCCCAGT GCTGCAATGA
1151 TACCGCGAGA CCCACGCTCA CCGGCTCCAG
          ATTTATCAGC AATAAACCAG
1201 CCAGCCGGAA GGGCCGAGCG CAGAAGTGGT
          CCTGCAACTT TATCCGCCTC
1251 CATCCAGTCT ATTAATTGTT GCCGGGAAGC
          TAGAGTAAGT AGTTCGCCAG
1301 TTAATAGTTT GCGCAACGTT GTTGCCATTG
          CTACAGGCAT CGTGGTGTCA
1351 CGCTCGTCGT TTGGTATGGC TTCATTCAGC
          TCCGGTTCCC AACGATCAAG
1401 GCGAGTTACA TGATCCCCCA TGTTGTGCAA
          AAAAGCGGTT AGCTCCTTCG
1451 GTCCTCCGAT CGTTGTCAGA AGTAAGTTGG
          CCGCAGTGTT ATCACTCATG
1501 GTTATGGCAG CACTGCATAA TTCTCTTACT
          GTCATGCCAT CCGTAAGATG
1551 CTTTTCTGTG ACTGGTGAGT ACTCAACCAA
          GTCATTCTGA GAATAGTGTA
1601 TGCGGCGACC GAGTTGCTCT TGCCCGGCGT
          CAATACGGGA TAATACCGCG
1651 CCACATAGCA GAACTTTAAA AGTGCTCATC
          ATTGGAAAAC GTTCTTCGGG
1701 GCGAAAACTC TCAAGGATCT TACCGCTGTT
          GAGATCCAGT TCGATGTAAC
1751 CCACTCGTGC ACCCAACTGA TCTTCAGCAT
          CTTTTACTTT CACCAGCGTT
1801 TCTGGGTGAG CAAAAACAGG AAGGCAAAAT
          GCCGCAAAAA AGGGAATAAG
1851 GGCGACACGG AAATGTTGAA TACTCATACT
          CTTCCTTTTT CAATATTATT
1901 GAAGCATTTA TCAGGGTTAT TGTCTCATGA
          GCGGATACAT ATTTGAATGT
1951 ATTTAGAAAA ATAAACAAAT AGGGGTTCCG
          CGCACATTTC CCCGAAAAGT
2001 GCCACCTGAC GTCTAAGAAA CCATTATTAT
          CATGACATTA ACCTATAAAA
2051 ATAGGCGTAT CACGAGGCCC TTTCGTCTCG
          CGCGTTTCGG TGATGACGGT
2101 GAAAACCTCT GACACATGCA GCTCCCGGAG
          ACGGTCACAG CTTGTCTGTA
2151 AGCGGATGCC GGGAGCAGAC AAGCCCGTCA
          GGGCGCGTCA GCGGGTGTTG
2201 GCGGGTGTCG GGGCTGGCTT AACTATGCGG
          CATCAGAGCA GATTGTACTG
2251 AGAGTGCACC ATATGCGGTG TGAAATACCG
          CACAGATGCG TAAGGAGAAA
2301 ATACCGCATC AGGCGATTCC AACATCCAAT
          AAATCATACA GGCAAGGCAA
2351 AGAATTAGCA AAATTAAGCA ATAAAGCCTC
          AGAGCATAAA GCTAAATCGG
2401 TTGTACCAAA AACATTATGA CCCTGTAATA
          CTTTTGCGGG AGAAGCCTTT
2451 ATTTCAACGC AAGGATAAAA ATTTTTAGAA
          CCCTCATATA TTTTAAATGC
2501 AATGCCTGAG TAATGTGTAG GTAAAGATTC
          AAACGGGTGA GAAAGGCCGG
2551 AGACAGTCAA ATCACCATCA ATATGATATT
          CAACCGTTCT AGCTGATAAA
2601 TTCATGCCGG AGAGGGTAGC TATTTTTGAG
          AGGTCTCTAC AAAGGCTATC
2651 AGGTCATTGC CTGAGAGTCT GGAGCAAACA
          AGAGAATCGA TGAACGGTAA
2701 TCGTAAAACT AGCATGTCAA TCATATGTAC
          CCCGGTTGAT AATCAGAAAA
2751 GCCCCAAAAA CAGGAAGATT GTATAAGCAA
          ATATTTAAAT TGTAAGCGTT
2801 AATATTTTGT TAAAATTCGC GTTAAATTTT
          TGTTAAATCA GCTCATTTTT
2851 TAACCAATAG GCCGAAATCG GCAAAATCCC
          TTATAAATCA AAAGAATAGA
2901 CCGAGATAGG GTTGAGTGTT GTTCCAGTTT
          GGAACAAGAG TCCACTATTA
2951 AAGAACGTGG ACTCCAACGT CAAAGGGCGA
          AAAACCGTCT ATCAGGGCGA
3001 TGGCCCACTA CGTGAACCAT CACCCTAATC
          AAGTTTTTTG GGGTCGAGGT
3051 GCCGTAAAGC ACTAAATCGG AACCCTAAAG
          GGAGCCCCCG ATTTAGAGCT
3101 TGACGGGGAA AGCCGGCGAA CGTGGCGAGA
          AAGGAAGGGA AGAAAGCGAA
3151 AGGAGCGGGC GCTAGGGCGC TGGCAAGTGT
          AGCGGTCACG CTGCGCGTAA
3201 CCACCACACC CGCCGCGCTT AATGCGCCGC
          TACAGGGCGC GTACTATGGT
3251 TGCTTTGACG AGCACGTATA ACGTGCTTTC
          CTCGTTAGAA TCAGAGCGGG
3301 AGCTAAACAG GAGGCCGATT AAAGGGATTT
          TAGACAGGAA CGGTACGCCA
3351 GAATCCTGAG AAGTGTTTTT ATAATCAGTG
          AGGCCACCGA GTAAAAGAGT
3401 CTGTCCATCA CGCAAATTAA CCGTTGTCGC
          AATACTTCTT TGATTAGTAA
3451 TAACATCACT TGCCTGAGTA GAAGAACTCA
          AACTATCGGC CTTGCTGGTA
3501 ATATCCAGAA CAATATTACC GCCAGCCATT
          GCAACGGAAT CGCCATTCGC
3551 CATTCAGGCT GCGCAACTGT TGGGAAGGGC
          GATCGGTGCG GGCCTCTTCC
3601 ACTGAGGCCC AGCTGCGCGC TCGCTCGCTC
          ACTGAGGCCG CCCGGGCAAA
3651 GCCCGGGCGT CGGGCGACCT TTGGTCGCCC
          GGCCTCAGTG AGCGAGCGAG
3701 CGCGCAGAGA GGGAGTGGCC AACTCCATCA
          CTAGGGGTTC CTTGTAGTTA
3751 ATGATTAACC CGCCATGCTA CTTATCTACT
          CGACATTGAT TATTGACTAG
3801 TTATTAATAG TAATCAATTA CGGGGTCATT
          AGTTCATAGC CCATATATGG
3851 AGTTCCGCGT TACATAACTT ACGGTAAATG
          GCCCGCCTGG CTGACCGCCC
3901 AACGACCCCC GCCCATTGAC GTCAATAATG
          ACGTATGTTC CCATAGTAAC
3951 GCCAATAGGG ACTTTCCATT GACGTCAATG
          GGTGGAGTAT TTACGGTAAA
4001 CTGCCCACTT GGCAGTACAT CAAGTGTATC
          ATATGCCAAG TACGCCCCCT
4051 ATTGACGTCA ATGACGGTAA ATGGCCCGCC
          TGGCATTATG CCCAGTACAT
4101 GACCTTATGG GACTTTCCTA CTTGGCAGTA
          CATCTACGTA TTAGTCATCG
4151 CTATTACCAT GGTCGAGGTG AGCCCCACGT
          TCTGCTTCAC TCTCCCCATC
4201 TCCCCCCCCT CCCCACCCCC AATTTTGTAT
          TTATTTATTT TTTAATTATT
4251 TTGTGCAGCG ATGGGGGCGG GGGGGGGGGG
          GGGGCGCGCG CCAGGCGGGG
4301 CGGGGCGGGG CGAGGGGCGG GGCGGGGCGA
          GGCGGAGAGG TGCGGCGGCA
4351 GCCAATCAGA GCGGCGCGCT CCGAAAGTTT
          CCTTTTATGG CGAGGCGGCG
4401 GCGGCGGCGG CCCTATAAAA AGCGAAGCGC
          GCGGCGGGCG GGAGTCGCTG
4451 CGTTGCCTTC GCCCCGTGCC CCGCTCCGCG
          CCGCCTCGCG CCGCCCGCCC
4501 CGGCTCTGAC TGACCGCGTT ACTCCCACAG
          GTGAGCGGGC GGGACGGCCC
4551 TTCTCCTCCG GGCTGTAATT AGCGCTTGGT
          TTAATGACGG CTTGTTTCTT
4601 TTCTGTGGCT GCGTGAAAGC CTTGAGGGGC
          TCCGGGAGGG CCCTTTGTGC
4651 GGGGGGAGCG GCTCGGGGGG TGCGTGCGTG
          TGTGTGTGCG TGGGGAGCGC
4701 CGCGTGCGGC TCCGCGCTGC CCGGCGGCTG
          TGAGCGCTGC GGGCGCGGCG
4751 CGGGGCTTTG TGCGCTCCGC AGTGTGCGCG
          AGGGGAGCGC GGCCGGGGGC
4801 GGTGCCCCGC GGTGCGGGGG GCTGCGAGGG
          GAACAAAGGC TGCGTGCGGG
4851 GTGTGTGCGT GGGGGGGTGA GCAGGGGGTG
          TGGGCGCGTC GGTCGGGCTG
4901 CAACCCCCCC TGCACCCCCC TCCCCGAGTT
          GCTGAGCACG GCCCGGCTTC
4951 GGGTGCGGGG CTCCGTACGG GGCGTGGCGC
          GGGGCTCGCC GTGCCGGGCG
5001 GGGGGTGGCG GCAGGTGGGG GTGCCGGGCG
          GGGCGGGGCC GCCTCGGGCC
5051 GGGGAGGGCT CGGGGGAGGG GCGCGGCGGC
          CCCCGGAGCG CCGGCGGCTG
5101 TCGAGGCGCG GCGAGCCGCA GCCATTGCCT
          TTTATGGTAA TCGTGCGAGA
5151 GGGCGCAGGG ACTTCCTTTG TCCCAAATCT
          GTGCGGAGCC GAAATCTGGG
5201 AGGCGCCGCC GCACCCCCTC TAGCGGGCGC
          GGGGCGAAGC GGTGCGGCGC
5251 CGGCAGGAAG GAAATGGGCG GGGAGGGCCT
          TCGTGCGTCG CCGCGCCGCC
5301 GTCCCCTTCT CCCTCTCCAG CCTCGGGGCT
          GTCCGCGGGG GGACGGCTGC
5351 CTTCGGGGGG GACGGGGCAG GGCGGGGTTC
          GGCTTCTGGC GTGTGACCGG
5401 CGGCTCTAGA GCCTCTGCTA ACCATGTTCA
          TGCCTTCTTC TTTTTCCTAC
5451 AGCTCCTGGG CAACGTGCTG GTTATTGTGC
          TGTCTCATCA TTTTGGCAAA
5501 GAATTGATTA ATTCGAGCGA ACGCGTCGAG
          TCGCTCGGTA CGATTTAAAT
5551 TGAATTGGCC TCGAGCGCAA GCTTGAGCTA
          GGACCTTCTG CCATGGCCCT
5601 GTGGATGCGC CTCCTGCCCC TGCTGGCGCT
          GCTGGCCCTC TGGGGACCTG
5651 ACCCAGCCGC AGCCTTTGTG AACCAACACC
          TGTGCGGCTC AGATCTGGTG
5701 GAAGCTCTCT ACCTAGTGTG CGGGGAACGA
          GGCTTCTTCT ACACACCCAG
5751 GACCAAGCGG GAGGCAGAGG ACCTGCAGGT
          GGGGCAGGTG GAGCTGGGCG
5801 GGGGCCCTGG TGCAGGCAGC CTGCAGCCCT
          TGGCCCTGGA GGGGTCGCGA
5851 CAGAAGCGTG GCATTGTGGA ACAATGCTGT
          ACCAGCATCT GCTCCCTCTA
5901 CCAGCTGGAG AACTACTGCA ACTAGACGCA
          GCCGTCGGCC GCTAATTCTA
5951 GATCGCGAAC AAACACCATT GTCACACTCC
          AGTATACACA AACACCATTG
6001 TCACACTCCA GATATCACAA ACACCATTGT
          CACACTCCAA GGCGAACAAA
6051 CACCATTGTC ACACTCCAAG GCTATTCTAG
          ATCGCGAATT ACATACTTCT
6101 TTACATTCCA GTATACATTA CATACTTCTT
          TACATTCCAG ATATCATTAC
6151 ATACTTCTTT ACATTCCAAG GCGAATTACA
          TACTTCTTTA CATTCCAAGG
6201 CTACCTGAGG CCCGGGGGTA CCTCTTAATT
          AACTGGCCTC ATGGGCCTTC
6251 CGCTCACTGC CCGCTTTCCA GTCGGGAAAC
          CTGTCGTGCC AGTCAGGTGC
6301 AGGCTGCCTA TCAGAAGGTG GTGGCTGGTG
          TGGCCAATGC CCTGGCTCAC
6351 AAATACCACT GAGATCTTTT TCCCTCTGCC
          AAAAATTATG GGGACATCAT
6401 GAAGCCCCTT GAGCATCTGA CTTCTGGCTA
          ATAAAGGAAA TTTATTTTCA
6451 TTGCAATAGT GTGTTGGAAT TTTTTGTGTC
          TCTCACTCGG AAGGACATAT
6501 GGGAGGGCAA ATCATTTAAA ACATCAGAAT
          GAGTATTTGG TTTAGAGTTT
6551 GGCAACATAT GCCCATATGC TGGCTGCCAT
          GAACAAAGGT TGGCTATAAA
6601 GAGGTCATCA GTATATGAAA CAGCCCCCTG
          CTGTCCATTC CTTATTCCAT
6651 AGAAAAGCCT TGACTTGAGG TTAGATTTTT
          TTTATATTTT GTTTTGTGTT
6701 ATTTTTTTCT TTAACATCCC TAAAATTTTC
          CTTACATGTT TTACTAGCCA
6751 GATTTTTCCT CCTCTCCTGA CTACTCCCAG
          TCATAGCTGT CCCTCTTCTC
6801 TTATGGAGAT CCCTCGACCT GCAGCCCAAG
          CTGTAGATAA GTAGCATGGC
6851 GGGTTAATCA TTAACTACAA GGAACCCCTA
          GTGATGGAGT TGGCCACTCC
6901 CTCTCTGCGC GCTCGCTCGC TCACTGAGGC
          CGGGCGACCA AAGGTCGCCC
6951 GACGCCCGGG CTTTGCCCGG GCGGCCTCAG
          TGAGCGAGCG AGCGCGCAGC
AAV2 5′ ITR: 3612-3742 bp
CAG promoter: 3779-5423 bp
hInsulin (hIns) : 5586-5932 bp
dmiRT (4 copies of the miRT-122a and
4 copies of the miRT-1): 5943-6203 bp
Rabbit β-globin polyA signal
(3′ UTR and 3′ flanking region of
rabbit beta-globin, including polyA
signal): 6293-6811 bp
AAV2 3′ ITR: 6870-7000 bp
pAAV-CAG-hInsAsp (SEQ ID NO: 49)
      1 AGTGAGCGAG CGAGCGCGCA GCTGCATTAA
          TGAATCGGCC AACGCGCGGG GAGAGGCGGT
    61 TTGCGTATTG GGCGCTCTTC CGCTTCCTCG
          CTCACTGACT CGCTGCGCTC GGTCGTTCGG
  121 CTGCGGCGAG CGGTATCAGC TCACTCAAAG
          GCGGTAATAC GGTTATCCAC AGAATCAGGG
  181 GATAACGCAG GAAAGAACAT GTGAGCAAAA
          GGCCAGCAAA AGGCCAGGAA CCGTAAAAAG
  241 GCCGCGTTGC TGGCGTTTTT CCATAGGCTC
          CGCCCCCCTG ACGAGCATCA CAAAAATCGA
  301 CGCTCAAGTC AGAGGTGGCG AAACCCGACA
          GGACTATAAA GATACCAGGC GTTTCCCCCT
  361 GGAAGCTCCC TCGTGCGCTC TCCTGTTCCG
          ACCCTGCCGC TTACCGGATA CCTGTCCGCC
  421 TTTCTCCCTT CGGGAAGCGT GGCGCTTTCT
          CATAGCTCAC GCTGTAGGTA TCTCAGTTCG
  481 GTGTAGGTCG TTCGCTCCAA GCTGGGCTGT
          GTGCACGAAC CCCCCGTTCA GCCCGACCGC
  541 TGCGCCTTAT CCGGTAACTA TCGTCTTGAG
          TCCAACCCGG TAAGACACGA CTTATCGCCA
  601 CTGGCAGCAG CCACTGGTAA CAGGATTAGC
          AGAGCGAGGT ATGTAGGCGG TGCTACAGAG
  661 TTCTTGAAGT GGTGGCCTAA CTACGGCTAC
          ACTAGAAGAA CAGTATTTGG TATCTGCGCT
  721 CTGCTGAAGC CAGTTACCTT CGGAAAAAGA
          GTTGGTAGCT CTTGATCCGG CAAACAAACC
  781 ACCGCTGGTA GCGGTGGTTT TTTTGTTTGC
          AAGCAGCAGA TTACGCGCAG AAAAAAAGGA
  841 TCTCAAGAAG ATCCTTTGAT CTTTTCTACG
          GGGTCTGACG CTCAGTGGAA CGAAAACTCA
  901 CGTTAAGGGA TTTTGGTCAT GAGATTATCA
          AAAAGGATCT TCACCTAGAT CCTTTTAAAT
  961 TAAAAATGAA GTTTTAAATC AATCTAAAGT
          ATATATGAGT AAACTTGGTC TGACAGTTAC
1021 CAATGCTTAA TCAGTGAGGC ACCTATCTCA
          GCGATCTGTC TATTTCGTTC ATCCATAGTT
1081 GCCTGACTCC CCGTCGTGTA GATAACTACG
          ATACGGGAGG GCTTACCATC TGGCCCCAGT
1141 GCTGCAATGA TACCGCGAGA CCCACGCTCA
          CCGGCTCCAG ATTTATCAGC AATAAACCAG
1201 CCAGCCGGAA GGGCCGAGCG CAGAAGTGGT
          CCTGCAACTT TATCCGCCTC CATCCAGTCT
1261 ATTAATTGTT GCCGGGAAGC TAGAGTAAGT
          AGTTCGCCAG TTAATAGTTT GCGCAACGTT
1321 GTTGCCATTG CTACAGGCAT CGTGGTGTCA
          CGCTCGTCGT TTGGTATGGC TTCATTCAGC
1381 TCCGGTTCCC AACGATCAAG GCGAGTTACA
          TGATCCCCCA TGTTGTGCAA AAAAGCGGTT
1441 AGCTCCTTCG GTCCTCCGAT CGTTGTCAGA
          AGTAAGTTGG CCGCAGTGTT ATCACTCATG
1501 GTTATGGCAG CACTGCATAA TTCTCTTACT
          GTCATGCCAT CCGTAAGATG CTTTTCTGTG
1561 ACTGGTGAGT ACTCAACCAA GTCATTCTGA
          GAATAGTGTA TGCGGCGACC GAGTTGCTCT
1621 TGCCCGGCGT CAATACGGGA TAATACCGCG
          CCACATAGCA GAACTTTAAA AGTGCTCATC
1681 ATTGGAAAAC GTTCTTCGGG GCGAAAACTC
          TCAAGGATCT TACCGCTGTT GAGATCCAGT
1741 TCGATGTAAC CCACTCGTGC ACCCAACTGA
          TCTTCAGCAT CTTTTACTTT CACCAGCGTT
1801 TCTGGGTGAG CAAAAACAGG AAGGCAAAAT
          GCCGCAAAAA AGGGAATAAG GGCGACACGG
1861 AAATGTTGAA TACTCATACT CTTCCTTTTT
          CAATATTATT GAAGCATTTA TCAGGGTTAT
1921 TGTCTCATGA GCGGATACAT ATTTGAATGT
          ATTTAGAAAA ATAAACAAAT AGGGGTTCCG
1981 CGCACATTTC CCCGAAAAGT GCCACCTGAC
          GTCTAAGAAA CCATTATTAT CATGACATTA
2041 ACCTATAAAA ATAGGCGTAT CACGAGGCCC
          TTTCGTCTCG CGCGTTTCGG TGATGACGGT
2101 GAAAACCTCT GACACATGCA GCTCCCGGAG
          ACGGTCACAG CTTGTCTGTA AGCGGATGCC
2161 GGGAGCAGAC AAGCCCGTCA GGGCGCGTCA
          GCGGGTGTTG GCGGGTGTCG GGGCTGGCTT
2221 AACTATGCGG CATCAGAGCA GATTGTACTG
          AGAGTGCACC ATATGCGGTG TGAAATACCG
2281 CACAGATGCG TAAGGAGAAA ATACCGCATC
          AGGCGATTCC AACATCCAAT AAATCATACA
2341 GGCAAGGCAA AGAATTAGCA AAATTAAGCA
          ATAAAGCCTC AGAGCATAAA GCTAAATCGG
2401 TTGTACCAAA AACATTATGA CCCTGTAATA
          CTTTTGCGGG AGAAGCCTTT ATTTCAACGC
2461 AAGGATAAAA ATTTTTAGAA CCCTCATATA
          TTTTAAATGC AATGCCTGAG TAATGTGTAG
2521 GTAAAGATTC AAACGGGTGA GAAAGGCCGG
          AGACAGTCAA ATCACCATCA ATATGATATT
2581 CAACCGTTCT AGCTGATAAA TTCATGCCGG
          AGAGGGTAGC TATTTTTGAG AGGTCTCTAC
2641 AAAGGCTATC AGGTCATTGC CTGAGAGTCT
          GGAGCAAACA AGAGAATCGA TGAACGGTAA
2701 TCGTAAAACT AGCATGTCAA TCATATGTAC
          CCCGGTTGAT AATCAGAAAA GCCCCAAAAA
2761 CAGGAAGATT GTATAAGCAA ATATTTAAAT
          TGTAAGCGTT AATATTTTGT TAAAATTCGC
2821 GTTAAATTTT TGTTAAATCA GCTCATTTTT
          TAACCAATAG GCCGAAATCG GCAAAATCCC
2881 TTATAAATCA AAAGAATAGA CCGAGATAGG
          GTTGAGTGTT GTTCCAGTTT GGAACAAGAG
2941 TCCACTATTA AAGAACGTGG ACTCCAACGT
          CAAAGGGCGA AAAACCGTCT ATCAGGGCGA
3001 TGGCCCACTA CGTGAACCAT CACCCTAATC
          AAGTTTTTTG GGGTCGAGGT GCCGTAAAGC
3061 ACTAAATCGG AACCCTAAAG GGAGCCCCCG
          ATTTAGAGCT TGACGGGGAA AGCCGGCGAA
3121 CGTGGCGAGA AAGGAAGGGA AGAAAGCGAA
          AGGAGCGGGC GCTAGGGCGC TGGCAAGTGT
3181 AGCGGTCACG CTGCGCGTAA CCACCACACC
          CGCCGCGCTT AATGCGCCGC TACAGGGCGC
3241 GTACTATGGT TGCTTTGACG AGCACGTATA
          ACGTGCTTTC CTCGTTAGAA TCAGAGCGGG
3301 AGCTAAACAG GAGGCCGATT AAAGGGATTT
          TAGACAGGAA CGGTACGCCA GAATCCTGAG
3361 AAGTGTTTTT ATAATCAGTG AGGCCACCGA
          GTAAAAGAGT CTGTCCATCA CGCAAATTAA
3421 CCGTTGTCGC AATACTTCTT TGATTAGTAA
          TAACATCACT TGCCTGAGTA GAAGAACTCA
3481 AACTATCGGC CTTGCTGGTA ATATCCAGAA
          CAATATTACC GCCAGCCATT GCAACGGAAT
3541 CGCCATTCGC CATTCAGGCT GCGCAACTGT
          TGGGAAGGGC GATCGGTGCG GGCCTCTTCC
3601 ACTGAGGCCC AGCTGCGCGC TCGCTCGCTC
          ACTGAGGCCG CCCGGGCAAA GCCCGGGCGT
3661 CGGGCGACCT TTGGTCGCCC GGCCTCAGTG
          AGCGAGCGAG CGCGCAGAGA GGGAGTGGCC
3721 AACTCCATCA CTAGGGGTTC CTTGTAGTTA
          ATGATTAACC CGCCATGCTA CTTATCTACT
3781 CGACATTGAT TATTGACTAG TTATTAATAG
          TAATCAATTA CGGGGTCATT AGTTCATAGC
3841 CCATATATGG AGTTCCGCGT TACATAACTT
          ACGGTAAATG GCCCGCCTGG CTGACCGCCC
3901 AACGACCCCC GCCCATTGAC GTCAATAATG
          ACGTATGTTC CCATAGTAAC GCCAATAGGG
3961 ACTTTCCATT GACGTCAATG GGTGGAGTAT
          TTACGGTAAA CTGCCCACTT GGCAGTACAT
4021 CAAGTGTATC ATATGCCAAG TACGCCCCCT
          ATTGACGTCA ATGACGGTAA ATGGCCCGCC
4081 TGGCATTATG CCCAGTACAT GACCTTATGG
          GACTTTCCTA CTTGGCAGTA CATCTACGTA
4141 TTAGTCATCG CTATTACCAT GGTCGAGGTG
          AGCCCCACGT TCTGCTTCAC TCTCCCCATC
4201 TCCCCCCCCT CCCCACCCCC AATTTTGTAT
          TTATTTATTT TTTAATTATT TTGTGCAGCG
4261 ATGGGGGCGG GGGGGGGGGG GGGGCGCGCG
          CCAGGCGGGG CGGGGCGGGG CGAGGGGCGG
4321 GGCGGGGCGA GGCGGAGAGG TGCGGCGGCA
          GCCAATCAGA GCGGCGCGCT CCGAAAGTTT
4381 CCTTTTATGG CGAGGCGGCG GCGGCGGCGG
          CCCTATAAAA AGCGAAGCGC GCGGCGGGCG
4441 GGAGTCGCTG CGTTGCCTTC GCCCCGTGCC
          CCGCTCCGCG CCGCCTCGCG CCGCCCGCCC
4501 CGGCTCTGAC TGACCGCGTT ACTCCCACAG
          GTGAGCGGGC GGGACGGCCC TTCTCCTCCG
4561 GGCTGTAATT AGCGCTTGGT TTAATGACGG
          CTTGTTTCTT TTCTGTGGCT GCGTGAAAGC
4621 CTTGAGGGGC TCCGGGAGGG CCCTTTGTGC
          GGGGGGAGCG GCTCGGGGGG TGCGTGCGTG
4681 TGTGTGTGCG TGGGGAGCGC CGCGTGCGGC
          TCCGCGCTGC CCGGCGGCTG TGAGCGCTGC
4741 GGGCGCGGCG CGGGGCTTTG TGCGCTCCGC
          AGTGTGCGCG AGGGGAGCGC GGCCGGGGGC
4801 GGTGCCCCGC GGTGCGGGGG GCTGCGAGGG
          GAACAAAGGC TGCGTGCGGG GTGTGTGCGT
4861 GGGGGGGTGA GCAGGGGGTG TGGGCGCGTC
          GGTCGGGCTG CAACCCCCCC TGCACCCCCC
4921 TCCCCGAGTT GCTGAGCACG GCCCGGCTTC
          GGGTGCGGGG CTCCGTACGG GGCGTGGCGC
4981 GGGGCTCGCC GTGCCGGGCG GGGGGTGGCG
          GCAGGTGGGG GTGCCGGGCG GGGCGGGGCC
5041 GCCTCGGGCC GGGGAGGGCT CGGGGGAGGG
          GCGCGGCGGC CCCCGGAGCG CCGGCGGCTG
5101 TCGAGGCGCG GCGAGCCGCA GCCATTGCCT
          TTTATGGTAA TCGTGCGAGA GGGCGCAGGG
5161 ACTTCCTTTG TCCCAAATCT GTGCGGAGCC
          GAAATCTGGG AGGCGCCGCC GCACCCCCTC
5221 TAGCGGGCGC GGGGCGAAGC GGTGCGGCGC
          CGGCAGGAAG GAAATGGGCG GGGAGGGCCT
5281 TCGTGCGTCG CCGCGCCGCC GTCCCCTTCT
          CCCTCTCCAG CCTCGGGGCT GTCCGCGGGG
5341 GGACGGCTGC CTTCGGGGGG GACGGGGCAG
          GGCGGGGTTC GGCTTCTGGC GTGTGACCGG
5401 CGGCTCTAGA GCCTCTGCTA ACCATGTTCA
          TGCCTTCTTC TTTTTCCTAC AGCTCCTGGG
5461 CAACGTGCTG GTTATTGTGC TGTCTCATCA
          TTTTGGCAAA GAATTGATTA ATTCGAGCGA
5521 ACGCGTCGAG TCGCTCGGTA CGATTTAAAT
          TGAATTGGCC TCGAGCGCAA GCTTGAGCTA
5581 GCGTCGACCT TCTGCCATGG CCCTGTGGAT
          GCGCCTCCTG CCCCTGCTGG CGCTGCTGGC
5641 CCTCTGGGGA CCTGACCCAG CCGCAGCCTT
          TGTGAACCAA CACCTGTGCG GCTCAGATCT
5701 GGTGGAAGCT CTCTACCTAG TGTGCGGGGA
          ACGAGGCTTC TTCTACACAC CCAGGACCAA
5761 GCGGGAGGCA GAGGACCTGC AGGTGGGGCA
          GGTGGAGCTG GGCGGGGGCC CTGGTGCAGG
5821 CAGCCTGCAG CCCTTGGCCC TGGAGGGGTC
          GCGACAGAAG CGTGGCATTG TGGAACAATG
5881 CTGTACCAGC ATCTGCTCCC TCTACCAGCT
          GGAGAACTAC TGCAACTAGA CGCAGCCGTC
5941 GACGGTACCC CCGACGCGGC CTAACTGGCC
          TCATGGGCCT TCCGCTCACT GCCCGCTTTC
6001 CAGTCGGGAA ACCTGTCGTG CCAGTCAGGT
          GCAGGCTGCC TATCAGAAGG TGGTGGCTGG
6061 TGTGGCCAAT GCCCTGGCTC ACAAATACCA
          CTGAGATCTT TTTCCCTCTG CCAAAAATTA
6121 TGGGGACATC ATGAAGCCCC TTGAGCATCT
          GACTTCTGGC TAATAAAGGA AATTTATTTT
6181 CATTGCAATA GTGTGTTGGA ATTTTTTGTG
          TCTCTCACTC GGAAGGACAT ATGGGAGGGC
6241 AAATCATTTA AAACATCAGA ATGAGTATTT
          GGTTTAGAGT TTGGCAACAT ATGCCCATAT
6301 GCTGGCTGCC ATGAACAAAG GTTGGCTATA
          AAGAGGTCAT CAGTATATGA AACAGCCCCC
6361 TGCTGTCCAT TCCTTATTCC ATAGAAAAGC
          CTTGACTTGA GGTTAGATTT TTTTTATATT
6421 TTGTTTTGTG TTATTTTTTT CTTTAACATC
          CCTAAAATTT TCCTTACATG TTTTACTAGC
6481 CAGATTTTTC CTCCTCTCCT GACTACTCCC
          AGTCATAGCT GTCCCTCTTC TCTTATGGAG
6541 ATCCCTCGAC CTGCAGCCCA AGCTGTAGAT
          AAGTAGCATG GCGGGTTAAT CATTAACTAC
6601 AAGGAACCCC TAGTGATGGA GTTGGCCACT
          CCCTCTCTGC GCGCTCGCTC GCTCACTGAG
6661 GCCGGGCGAC CAAAGGTCGC CCGACGCCCG
          GGCTTTGCCC GGGCGGCCTC AGTGAGCGAG
6721 CGAGCGCGCA GCTGGCGTAA
AAV2 5′ ITR:3601-3742bp
CAG promoter: 3779-5423 bp
hInsulin Aspartic (hInsAsp): 5590-5936 bp
Rabbit β-globin polyA signal
(3′ UTR and 3′ flanking region
of rabbit beta-globin, including
polyA signal): 6025-6543 bp
AAV2 3′ ITR: 6602-6743 bp
pAAV-CAG-hInsWt (SEQ ID NO: 50)
      1 AGTGAGCGAG CGAGCGCGCA GCTGCATTAA
          TGAATCGGCC AACGCGCGGG GAGAGGCGGT
    61 TTGCGTATTG GGCGCTCTTC CGCTTCCTCG
          CTCACTGACT CGCTGCGCTC GGTCGTTCGG
  121 CTGCGGCGAG CGGTATCAGC TCACTCAAAG
          GCGGTAATAC GGTTATCCAC AGAATCAGGG
  181 GATAACGCAG GAAAGAACAT GTGAGCAAAA
          GGCCAGCAAA AGGCCAGGAA CCGTAAAAAG
  241 GCCGCGTTGC TGGCGTTTTT CCATAGGCTC
          CGCCCCCCTG ACGAGCATCA CAAAAATCGA
  301 CGCTCAAGTC AGAGGTGGCG AAACCCGACA
          GGACTATAAA GATACCAGGC GTTTCCCCCT
  361 GGAAGCTCCC TCGTGCGCTC TCCTGTTCCG
          ACCCTGCCGC TTACCGGATA CCTGTCCGCC
  421 TTTCTCCCTT CGGGAAGCGT GGCGCTTTCT
          CATAGCTCAC GCTGTAGGTA TCTCAGTTCG
  481 GTGTAGGTCG TTCGCTCCAA GCTGGGCTGT
          GTGCACGAAC CCCCCGTTCA GCCCGACCGC
  541 TGCGCCTTAT CCGGTAACTA TCGTCTTGAG
          TCCAACCCGG TAAGACACGA CTTATCGCCA
  601 CTGGCAGCAG CCACTGGTAA CAGGATTAGC
          AGAGCGAGGT ATGTAGGCGG TGCTACAGAG
  661 TTCTTGAAGT GGTGGCCTAA CTACGGCTAC
          ACTAGAAGAA CAGTATTTGG TATCTGCGCT
  721 CTGCTGAAGC CAGTTACCTT CGGAAAAAGA
          GTTGGTAGCT CTTGATCCGG CAAACAAACC
  781 ACCGCTGGTA GCGGTGGTTT TTTTGTTTGC
          AAGCAGCAGA TTACGCGCAG AAAAAAAGGA
  841 TCTCAAGAAG ATCCTTTGAT CTTTTCTACG
          GGGTCTGACG CTCAGTGGAA CGAAAACTCA
  901 CGTTAAGGGA TTTTGGTCAT GAGATTATCA
          AAAAGGATCT TCACCTAGAT CCTTTTAAAT
  961 TAAAAATGAA GTTTTAAATC AATCTAAAGT
          ATATATGAGT AAACTTGGTC TGACAGTTAC
1021 CAATGCTTAA TCAGTGAGGC ACCTATCTCA
          GCGATCTGTC TATTTCGTTC ATCCATAGTT
1081 GCCTGACTCC CCGTCGTGTA GATAACTACG
          ATACGGGAGG GCTTACCATC TGGCCCCAGT
1141 GCTGCAATGA TACCGCGAGA CCCACGCTCA
          CCGGCTCCAG ATTTATCAGC AATAAACCAG
1201 CCAGCCGGAA GGGCCGAGCG CAGAAGTGGT
          CCTGCAACTT TATCCGCCTC CATCCAGTCT
1261 ATTAATTGTT GCCGGGAAGC TAGAGTAAGT
          AGTTCGCCAG TTAATAGTTT GCGCAACGTT
1321 GTTGCCATTG CTACAGGCAT CGTGGTGTCA
          CGCTCGTCGT TTGGTATGGC TTCATTCAGC
1381 TCCGGTTCCC AACGATCAAG GCGAGTTACA
          TGATCCCCCA TGTTGTGCAA AAAAGCGGTT
1441 AGCTCCTTCG GTCCTCCGAT CGTTGTCAGA
          AGTAAGTTGG CCGCAGTGTT ATCACTCATG
1501 GTTATGGCAG CACTGCATAA TTCTCTTACT
          GTCATGCCAT CCGTAAGATG CTTTTCTGTG
1561 ACTGGTGAGT ACTCAACCAA GTCATTCTGA
          GAATAGTGTA TGCGGCGACC GAGTTGCTCT
1621 TGCCCGGCGT CAATACGGGA TAATACCGCG
          CCACATAGCA GAACTTTAAA AGTGCTCATC
1681 ATTGGAAAAC GTTCTTCGGG GCGAAAACTC
          TCAAGGATCT TACCGCTGTT GAGATCCAGT
1741 TCGATGTAAC CCACTCGTGC ACCCAACTGA
          TCTTCAGCAT CTTTTACTTT CACCAGCGTT
1801 TCTGGGTGAG CAAAAACAGG AAGGCAAAAT
          GCCGCAAAAA AGGGAATAAG GGCGACACGG
1861 AAATGTTGAA TACTCATACT CTTCCTTTTT
          CAATATTATT GAAGCATTTA TCAGGGTTAT
1921 TGTCTCATGA GCGGATACAT ATTTGAATGT
          ATTTAGAAAA ATAAACAAAT AGGGGTTCCG
1981 CGCACATTTC CCCGAAAAGT GCCACCTGAC
          GTCTAAGAAA CCATTATTAT CATGACATTA
2041 ACCTATAAAA ATAGGCGTAT CACGAGGCCC
          TTTCGTCTCG CGCGTTTCGG TGATGACGGT
2101 GAAAACCTCT GACACATGCA GCTCCCGGAG
          ACGGTCACAG CTTGTCTGTA AGCGGATGCC
2161 GGGAGCAGAC AAGCCCGTCA GGGCGCGTCA
          GCGGGTGTTG GCGGGTGTCG GGGCTGGCTT
2221 AACTATGCGG CATCAGAGCA GATTGTACTG
          AGAGTGCACC ATATGCGGTG TGAAATACCG
2281 CACAGATGCG TAAGGAGAAA ATACCGCATC
          AGGCGATTCC AACATCCAAT AAATCATACA
2341 GGCAAGGCAA AGAATTAGCA AAATTAAGCA
          ATAAAGCCTC AGAGCATAAA GCTAAATCGG
2401 TTGTACCAAA AACATTATGA CCCTGTAATA
          CTTTTGCGGG AGAAGCCTTT ATTTCAACGC
2461 AAGGATAAAA ATTTTTAGAA CCCTCATATA
          TTTTAAATGC AATGCCTGAG TAATGTGTAG
2521 GTAAAGATTC AAACGGGTGA GAAAGGCCGG
          AGACAGTCAA ATCACCATCA ATATGATATT
2581 CAACCGTTCT AGCTGATAAA TTCATGCCGG
          AGAGGGTAGC TATTTTTGAG AGGTCTCTAC
2641 AAAGGCTATC AGGTCATTGC CTGAGAGTCT
          GGAGCAAACA AGAGAATCGA TGAACGGTAA
2701 TCGTAAAACT AGCATGTCAA TCATATGTAC
          CCCGGTTGAT AATCAGAAAA GCCCCAAAAA
2761 CAGGAAGATT GTATAAGCAA ATATTTAAAT
          TGTAAGCGTT AATATTTTGT TAAAATTCGC
2821 GTTAAATTTT TGTTAAATCA GCTCATTTTT
          TAACCAATAG GCCGAAATCG GCAAAATCCC
2881 TTATAAATCA AAAGAATAGA CCGAGATAGG
          GTTGAGTGTT GTTCCAGTTT GGAACAAGAG
2941 TCCACTATTA AAGAACGTGG ACTCCAACGT
          CAAAGGGCGA AAAACCGTCT ATCAGGGCGA
3001 TGGCCCACTA CGTGAACCAT CACCCTAATC
          AAGTTTTTTG GGGTCGAGGT GCCGTAAAGC
3061 ACTAAATCGG AACCCTAAAG GGAGCCCCCG
          ATTTAGAGCT TGACGGGGAA AGCCGGCGAA
3121 CGTGGCGAGA AAGGAAGGGA AGAAAGCGAA
          AGGAGCGGGC GCTAGGGCGC TGGCAAGTGT
3181 AGCGGTCACG CTGCGCGTAA CCACCACACC
          CGCCGCGCTT AATGCGCCGC TACAGGGCGC
3241 GTACTATGGT TGCTTTGACG AGCACGTATA
          ACGTGCTTTC CTCGTTAGAA TCAGAGCGGG
3301 AGCTAAACAG GAGGCCGATT AAAGGGATTT
          TAGACAGGAA CGGTACGCCA GAATCCTGAG
3361 AAGTGTTTTT ATAATCAGTG AGGCCACCGA
          GTAAAAGAGT CTGTCCATCA CGCAAATTAA
3421 CCGTTGTCGC AATACTTCTT TGATTAGTAA
          TAACATCACT TGCCTGAGTA GAAGAACTCA
3481 AACTATCGGC CTTGCTGGTA ATATCCAGAA
          CAATATTACC GCCAGCCATT GCAACGGAAT
3541 CGCCATTCGC CATTCAGGCT GCGCAACTGT
          TGGGAAGGGC GATCGGTGCG GGCCTCTTCC
3601 ACTGAGGCCC AGCTGCGCGC TCGCTCGCTC
          ACTGAGGCCG CCCGGGCAAA GCCCGGGCGT
3661 CGGGCGACCT TTGGTCGCCC GGCCTCAGTG
          AGCGAGCGAG CGCGCAGAGA GGGAGTGGCC
3721 AACTCCATCA CTAGGGGTTC CTTGTAGTTA
          ATGATTAACC CGCCATGCTA CTTATCTACT
3781 CGACATTGAT TATTGACTAG TTATTAATAG
          TAATCAATTA CGGGGTCATT AGTTCATAGC
3841 CCATATATGG AGTTCCGCGT TACATAACTT
          ACGGTAAATG GCCCGCCTGG CTGACCGCCC
3901 AACGACCCCC GCCCATTGAC GTCAATAATG
          ACGTATGTTC CCATAGTAAC GCCAATAGGG
3961 ACTTTCCATT GACGTCAATG GGTGGAGTAT
          TTACGGTAAA CTGCCCACTT GGCAGTACAT
4021 CAAGTGTATC ATATGCCAAG TACGCCCCCT
          ATTGACGTCA ATGACGGTAA ATGGCCCGCC
4081 TGGCATTATG CCCAGTACAT GACCTTATGG
          GACTTTCCTA CTTGGCAGTA CATCTACGTA
4141 TTAGTCATCG CTATTACCAT GGTCGAGGTG
          AGCCCCACGT TCTGCTTCAC TCTCCCCATC
4201 TCCCCCCCCT CCCCACCCCC AATTTTGTAT
          TTATTTATTT TTTAATTATT TTGTGCAGCG
4261 ATGGGGGCGG GGGGGGGGGG GGGGCGCGCG
          CCAGGCGGGG CGGGGCGGGG CGAGGGGCGG
4321 GGCGGGGCGA GGCGGAGAGG TGCGGCGGCA
          GCCAATCAGA GCGGCGCGCT CCGAAAGTTT
4381 CCTTTTATGG CGAGGCGGCG GCGGCGGCGG
          CCCTATAAAA AGCGAAGCGC GCGGCGGGCG
4441 GGAGTCGCTG CGTTGCCTTC GCCCCGTGCC
          CCGCTCCGCG CCGCCTCGCG CCGCCCGCCC
4501 CGGCTCTGAC TGACCGCGTT ACTCCCACAG
          GTGAGCGGGC GGGACGGCCC TTCTCCTCCG
4561 GGCTGTAATT AGCGCTTGGT TTAATGACGG
          CTTGTTTCTT TTCTGTGGCT GCGTGAAAGC
4621 CTTGAGGGGC TCCGGGAGGG CCCTTTGTGC
          GGGGGGAGCG GCTCGGGGGG TGCGTGCGTG
4681 TGTGTGTGCG TGGGGAGCGC CGCGTGCGGC
          TCCGCGCTGC CCGGCGGCTG TGAGCGCTGC
4741 GGGCGCGGCG CGGGGCTTTG TGCGCTCCGC
          AGTGTGCGCG AGGGGAGCGC GGCCGGGGGC
4801 GGTGCCCCGC GGTGCGGGGG GCTGCGAGGG
          GAACAAAGGC TGCGTGCGGG GTGTGTGCGT
4861 GGGGGGGTGA GCAGGGGGTG TGGGCGCGTC
          GGTCGGGCTG CAACCCCCCC TGCACCCCCC
4921 TCCCCGAGTT GCTGAGCACG GCCCGGCTTC
          GGGTGCGGGG CTCCGTACGG GGCGTGGCGC
4981 GGGGCTCGCC GTGCCGGGCG GGGGGTGGCG
          GCAGGTGGGG GTGCCGGGCG GGGCGGGGCC
5041 GCCTCGGGCC GGGGAGGGCT CGGGGGAGGG
          GCGCGGCGGC CCCCGGAGCG CCGGCGGCTG
5101 TCGAGGCGCG GCGAGCCGCA GCCATTGCCT
          TTTATGGTAA TCGTGCGAGA GGGCGCAGGG
5161 ACTTCCTTTG TCCCAAATCT GTGCGGAGCC
          GAAATCTGGG AGGCGCCGCC GCACCCCCTC
5221 TAGCGGGCGC GGGGCGAAGC GGTGCGGCGC
          CGGCAGGAAG GAAATGGGCG GGGAGGGCCT
5281 TCGTGCGTCG CCGCGCCGCC GTCCCCTTCT
          CCCTCTCCAG CCTCGGGGCT GTCCGCGGGG
5341 GGACGGCTGC CTTCGGGGGG GACGGGGCAG
          GGCGGGGTTC GGCTTCTGGC GTGTGACCGG
5401 CGGCTCTAGA GCCTCTGCTA ACCATGTTCA
          TGCCTTCTTC TTTTTCCTAC AGCTCCTGGG
5461 CAACGTGCTG GTTATTGTGC TGTCTCATCA
          TTTTGGCAAA GAATTGATTA ATTCGAGCGA
5521 ACGCGTCGAG TCGCTCGGTA CGATTTAAAT
          TGAATTGGCC TCGAGCGCAA GCTTGAGCTA
5581 GCGTCGAGGG GTCGACATGG CCCTGTGGAT
          GCGCCTCCTG CCCCTGCTGG CGCTGCTGGC
5641 CCTCTGGGGA CCTGACCCAG CCGCAGCCTT
          TGTGAACCAA CACCTGTGCG GCTCACACCT
5701 GGTGGAAGCT CTCTACCTAG TGTGCGGGGA
          ACGAGGCTTC TTCTACACAC CCAGGACCAA
5761 GCGGGAGGCA GAGGACCTGC AGGTGGGGCA
          GGTGGAGCTG GGCGGGGGCC CTGGTGCAGG
5821 CAGCCTGCAG CCCTTGGCCC TGGAGGGGTC
          GCGACAGAAG CGTGGCATTG TGGAACAATG
5881 CTGTACCAGC ATCTGCTCCC TCTACCAGCT
          GGAGAACTAC TGCAACTAGG TCGACCCCTC
5941 GACGGTACCC CCGACGCGGC CTAACTGGCC
          TCATGGGCCT TCCGCTCACT GCCCGCTTTC
6001 CAGTCGGGAA ACCTGTCGTG CCAGTCAGGT
          GCAGGCTGCC TATCAGAAGG TGGTGGCTGG
6061 TGTGGCCAAT GCCCTGGCTC ACAAATACCA
          CTGAGATCTT TTTCCCTCTG CCAAAAATTA
6121 TGGGGACATC ATGAAGCCCC TTGAGCATCT
          GACTTCTGGC TAATAAAGGA AATTTATTTT
6181 CATTGCAATA GTGTGTTGGA ATTTTTTGTG
          TCTCTCACTC GGAAGGACAT ATGGGAGGGC
6241 AAATCATTTA AAACATCAGA ATGAGTATTT
          GGTTTAGAGT TTGGCAACAT ATGCCCATAT
6301 GCTGGCTGCC ATGAACAAAG GTTGGCTATA
          AAGAGGTCAT CAGTATATGA AACAGCCCCC
6361 TGCTGTCCAT TCCTTATTCC ATAGAAAAGC
          CTTGACTTGA GGTTAGATTT TTTTTATATT
6421 TTGTTTTGTG TTATTTTTTT CTTTAACATC
          CCTAAAATTT TCCTTACATG TTTTACTAGC
6481 CAGATTTTTC CTCCTCTCCT GACTACTCCC
          AGTCATAGCT GTCCCTCTTC TCTTATGGAG
6541 ATCCCTCGAC CTGCAGCCCA AGCTGTAGAT
          AAGTAGCATG GCGGGTTAAT CATTAACTAC
6601 AAGGAACCCC TAGTGATGGA GTTGGCCACT
          CCCTCTCTGC GCGCTCGCTC GCTCACTGAG
6661 GCCGGGCGAC CAAAGGTCGC CCGACGCCCG
          GGCTTTGCCC GGGCGGCCTC AGTGAGCGAG
6721 CGAGCGCGCA GCTGGCGTAA
AAV2 5′ ITR: 3601-3742 bp
CAG promoter: 3779-5423 bp
hInsulin Wild-type (hInsWt): 5597-5929 bp
Rabbit β-globin polyA signal
(3′ UTR and 3′ flanking
region of rabbit beta-globin, including
polyA signal): 6025-6543 bp
AAV2 3′ ITR: 6602-6743 bp
```

1. A method for the treatment and/or prevention of neuroinflammation, neurodegeneration and/or cognitive decline, or a disease or condition associated therewith, the method comprising administering in an subject in need thereof a gene construct comprising a nucleotide sequence encoding insulin.

2. The method of claim 1, wherein the nucleotide sequence encoding insulin is operably linked to a ubiquitous promoter.

3. The method of claim 1, wherein the ubiquitous promoter is selected from the group consisting of a CAG promoter and a CMV promoter, preferably wherein the ubiquitous promoter is a CAG promoter.

4. The method of claim 1, wherein the gene construct comprises at least one target sequence of a microRNA expressed in a tissue where the expression of insulin is wanted to be prevented, preferably wherein the at least one target sequence of a microRNA is selected from those target sequences that bind to microRNAs expressed in heart and/or liver of the mammal.

5. The method of claim 4, wherein the gene construct comprises at least one target sequence of a microRNA expressed in the liver and at least one target sequence of a microRNA expressed in the heart, preferably wherein a target sequence of a microRNA expressed in the heart is selected from SEQ ID NO's: 8 and 16-20 and a target sequence of a microRNA expressed in the liver is selected from SEQ ID NO's: 7 and 9-15, more preferably wherein the gene construct comprises a target sequence of microRNA-122a (SEQ ID NO: 7) and a target sequence of microRNA-1 (SEQ ID NO: 8).

6. The method of claim 1, wherein the nucleotide sequence encoding insulin is selected from the group consisting of:
(a) a nucleotide sequence encoding a polypeptide comprising an amino acid sequence that has at least 60% sequence identity with the amino acid sequence of SEQ ID NO: 1, 2 or 3;
(b) a nucleotide sequence that has at least 60% sequence identity with the nucleotide sequence of SEQ ID NO: 4, 5 or 6; and
(c) a nucleotide sequence the sequence of which differs from the sequence of a nucleotide sequence of (b) due to the degeneracy of the genetic code.

7. The method of claim 1, wherein the gene construct is comprised in an expression vector.

8. The method of claim 7, wherein the expression vector is a viral vector, preferably wherein the expression vector is a viral vector selected from the group consisting of adenoviral vectors, adeno-associated viral vectors, retroviral vectors, and lentiviral vectors, more preferably wherein the expression vector is an adeno-associated viral vector.

9. The method of claim 8, wherein the expression vector is an adeno-associated viral vector of serotype 1, 2, 3, 4, 5, 6, 7, 8, 9, rh10, rh8, Cb4, rh74, DJ, 2/5, 2/1, 1/2 or Anc80, preferably wherein the expression vector is an adeno-associated viral vector of serotype 1, 2 or 9, more preferably wherein the expression vector is an adeno-associated viral vector of serotype 1 or 9.

10. The method of claim 1, wherein the gene construct is comprised in a pharmaceutical composition, together with one or more pharmaceutically acceptable ingredients.

11. The method of claim 1, wherein the disease or condition associated with neuroinflammation, neurodegeneration and/or cognitive disorder is selected from the group consisting of: a cognitive disorder, dementia, Alzheimer's disease, vascular dementia, Lewy body dementia, frontotemporal dementia (FTD), Parkinson's disease, Parkinson-like disease, Parkinsonism, Huntington's disease, traumatic brain injury, prion disease, dementia/neurocognitive issues due to HIV infection, dementia/neurocognitive issues due to aging, tauopathy, multiple sclerosis and other neuroinflammatory/neurodegenerative diseases, preferably Alzheimer's disease, Parkinson's disease and/or Parkinson-like disease, more preferably Alzheimer's disease or Parkinson's disease.

12. The method of claim 1, wherein the gene construct is administered by intra-CSF administration.

13. A gene construct comprising a nucleotide sequence encoding insulin wherein the nucleotide sequence encoding insulin is operably linked to a ubiquitous promoter and wherein the gene construct comprises at least one target sequence of a microRNA expressed in a tissue where the expression of insulin is wanted to be prevented, preferably wherein the at least one target sequence of a microRNA is selected from those target sequences that bind to microRNAs expressed in heart and/or liver of the mammal.

14. The gene construct of claim 13, wherein the gene construct comprises at least one target sequence of a microRNA expressed in the liver and at least one target sequence of a microRNA expressed in the heart, preferably wherein a target sequence of a microRNA expressed in the heart is selected from SEQ ID NO's: 8 and 16-20 and a target sequence of a microRNA expressed in the liver is selected from SEQ ID NO's: 7 and 9-15, more preferably wherein the gene construct comprises a target sequence of microRNA-122a (SEQ ID NO: 7) and a target sequence of microRNA-1 (SEQ ID NO: 8).

15. An expression vector comprising a gene construct as defined in claim 13, preferably wherein the expression vector is a viral vector, more preferably wherein the expression vector is a viral vector selected from the group consisting of adenoviral vectors, adeno-associated viral vectors, retroviral vectors, and lentiviral vectors, most preferably wherein the expression vector is an adeno-associated viral vector.

16. The method of claim 7, wherein the expression vector is comprised in a pharmaceutical composition, together with one or more pharmaceutically acceptable ingredients.


SEQUENCE LISTING (50 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 110 aa, Mus musculus
MALWMRFLPLLALLFLWESHPTQAFVKQHLCGSHLVEALYLVCGERGFFYTPMSRREVEDPQVAQLELGGGPGAGDLQTLALEVAQQKRGIVDQCCTSICSLYQLENYCN

SEQ ID NO: 3 - protein, 110 aa, Canis lupus familiaris
MALWMRLLPLLALLALWAPAPTRAFVNQHLCGSHLVEALYLVCGERGFFYTPKARREVEDLQVRDVELAGAPGEGGLQPLALEGALQKRGIVEQCCTSICSLYQLENYCN

SEQ ID NO: 4 - DNA, 333 bp, Homo sapiens
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 5 - DNA, 333 bp, Mus musculus
atggccctgtggatgcgcttcctgcccctgctggccctgctcttcctctgggagtcccaccccacccaggcttttgtcaagcagcacctttgtggttcccacctggtggaggctctctacctggtgtgtggggagcgtggcttcttctacacacccatgtcccgccgtgaagtggaggacccacaagtggcacaactggagctgggtggaggcccgggagcaggtgaccttcagaccttggcactggaggtggcccagcagaagcgtggcattgtagatcagtgctgcaccagcatctgctccctctaccagctggagaactactgcaactag

SEQ ID NO: 6 - DNA, 333 bp, Canis lupus familiaris
atggccctctggatgcgcctcctgcccctgctggccctgctggccctctgggcgcccgcgcccacccgagccttcgttaaccagcacctgtgtggctcccacctggtagaggctctgtacctggtgtgcggggagcgcggcttcttctacacgcctaaggcccgccgggaggtggaggacctgcaggtgagggacgtggagctggccggggcgcctggcgagggcggcctgcagcccctggccctggagggggccctgcagaagcgaggcatcgtggagcagtgctgcaccagcatctgctccctctaccagctggagaattactgcaactag

SEQ ID NO: 7 - DNA, 22 bp, Artificial sequence
  miRT-122a
caaacaccattgtcacactcca

SEQ ID NO: 8 - DNA, 22 bp, Artificial sequence
  miRT-1
ttacatacttctttacattcca

SEQ ID NO: 9 - DNA, 21 bp, Artificial sequence
  miRT-152
ccaagttctgtcatgcactga

SEQ ID NO: 10 - DNA, 23 bp, Artificial sequence
  miRT-199a-5p
gaacaggtagtctgaacactggg

SEQ ID NO: 11 - DNA, 22 bp, Artificial sequence
  miRT-199a-3p
taaccaatgtgcagactactgt

SEQ ID NO: 12 - DNA, 21 bp, Artificial sequence
  miRT-215
gtctgtcaattcataggtcat

SEQ ID NO: 13 - DNA, 21 bp, Artificial sequence
  miRT-192
ggctgtcaattcataggtcag

SEQ ID NO: 14 - DNA, 22 bp, Artificial sequence
  miRT-148a
acaaagttctgtagtgcactga

SEQ ID NO: 15 - DNA, 22 bp, Artificial sequence
  miRT-194
tccacatggagttgctgttaca

SEQ ID NO: 16 - DNA, 22 bp, Artificial sequence
  miRT-133a
cagctggttgaaggggaccaaa

SEQ ID NO: 17 - DNA, 22 bp, Artificial sequence
  miRT-206
ccacacacttccttacattcca

SEQ ID NO: 18 - DNA, 22 bp, Artificial sequence
  miRT-208a-5p
gtataacccgggccaaaagctc

SEQ ID NO: 19 - DNA, 22 bp, Artificial sequence
  miRT-208a-3p
acaagctttttgctcgtcttat

SEQ ID NO: 20 - DNA, 21 bp, Artificial sequence
  miRT-499-5p
aaacatcactgcaagtcttaa

SEQ ID NO: 21 - DNA, 133 bp, Artificial sequence
  chimeric intron composed of introns from human beta-globin and immunoglobulin heavy chain genes
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 22 - DNA, 1671 bp, Artificial sequence
  CAG promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 23 - DNA, 212 bp, Artificial sequence
  CMV promoter
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 24 - DNA, 380 bp, Artificial sequence
  CMV enhancer
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 25 - DNA, 411 bp, Artificial sequence
  mini-CMV promoter
tatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagctt

SEQ ID NO: 26 - DNA, 1178 bp, Artificial sequence
  EF1alpha promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 27 - DNA, 623 bp, Artificial sequence
  RSV promoter
catgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagct

SEQ ID NO: 28 - DNA, 448 bp, Artificial sequence
  Synapsin 1 promoter
ctgcgctctcaggcacgacacgactcctccgctgcccaccgcagactgaggcagcgctgagtcgccggcgccgcagcgcagatggtcgcgcccgtgcccccctatctcgcgcctcgcgtggtgcggtccggctgggccggcggcggcgcggacgcgaccaaggtggccgggaaggggagtttgcgggggaccggcgagtgacgtcagcgcgccttcagtgctgaggcggcggtggcgcgcgccgccaggcgggggcgaaggcactgtccgcggtgctgaagctggcagtgcgcacgcgcctcgccgcatcctgtttcccctccccctctctgataggggatgcgcaatttggggaatgggggttgggtgcttgtccagtgggtcggggtcggtcgtcaggtaggcacccccaccccgcctcatcctggtcctaaaacccacttgcact

SEQ ID NO: 29 - DNA, 1296 bp, Artificial sequence
  Calcium/calmodulin-dependent protein kinase II (CaMKII) promoter
taacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgggg

SEQ ID NO: 30 - DNA, 706 bp, Artificial sequence
  Glial fibrillary acidic protein (GFAP) promoter
cgcgtgatctaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggtctagagtcga

SEQ ID NO: 31 - DNA, 1153 bp, Artificial sequence
  Nestin promoter
gaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaaagatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggagacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctccgaaacgggccctct

SEQ ID NO: 32 - DNA, 438 bp, Artificial sequence
  Homeobox Protein 9 (HB9) promoter
tgaataaatttaagcaggctaattaatatataaactagctcaatttgtcaagttgatttgtattttagttaattgtgaaagtaattaccacatggtcaaattaacagctttctggaaatgaccaagcctgaggttttatttccttcctgggtgaagaaaattcatttttccaagctcttgatgtgatgaataaaagtcataaatctgggtgattggtgcaggcagagtctaaatggcttcatatttcattttaggtttaatagaaatattcatgctctgttttaatgaaattaaattgaagggggatggggctagagtggttagctgatgaattgacaaaaactaatcagctttattgggaaacaggtttaagggcacggacgtgtcaataacgctcagcctgaccccctcttccattagctaggcaggctgattaga

SEQ ID NO: 33 - DNA, 3015 bp, Artificial sequence
  Tyrosine hydroxylase (TH) promoter
ctgctaggggctgcttcccagctactcctcttggctccgtggcttgccttccagcctgtgtgctgtctggagagcctttaaagcctcacttccaccaactagaagtctctccccaaccctgccctgacctcaagtgcacctcttcaaagtcaggtttagcagctgcagctgggggccctgaatcccacccctgctgtcttccttgaagacagaagtgttgggagctgaggatctgggctagagactggctgtatgatccagagaagtagtgtgcttctgggcctcagatttcccttctgtagaacaggtttgtctgaaatggagaggttggtgctcctctgcagggcctagtgggagtcaccatgagtggttaaaagatccagcttgtcttttggtgagctttgagaggaggtaacagggctgagttctggaagcctgaccaagggcagacttaaggggcctcttggagttgttctcatcaaatggggatgggacacagctaaagtgcccagggcttctctgtgcccacagatgctttagatcttggcacagtgtggtctaccagctgtctctctctgtgtatatatatgtatttcatagacagtgtacagtggcctggtttgtgctatcaggctggatatggacagaggcaagagtttgtggcagcagttatctcccaagagagtccaaagacatcatgttttcaagtttaggccaggtgctacttgagagagctcagacacagacaaaggtctggagagcacatgtcctccacccccacctagcttctgttgcaagcacctccagccgagacaagagaacgaattaaaaagcaatatttgtgtcagtgtaagacatttgccgaaaggttaaatccacattcgtgttgctgcagagcagccccctatgcaggatttgttagatacagctccgtcctaccctgtgccagctgagcaaacgccaggctgggtggggtggaacccagcctgggtttgcctcaccctgcaatccccccagcaccctctaaaggaggaccctgtggtgggcatgcagacctagggactgggcatagataacctttgggtttgggcaacagcccccactcctcaggattgaaggctaaggtgcagccagctctgccttcatggtgggaatgtctccacgtgacccctttctgggctgtggagaacactcagagaagagtcctgggatgccaggcaggccagggatgtgctgggcatgttgagacaggagtgggctaagccagcagagttgctgacccaggaagagttcagaaaggggcatggaacatggggaggggtccatagtgagagagagcaggcagtgcagagtaaatagtccctgagctgggggttatgggatttgcaggagcttgctcagagaaggcagaggagagatgctgcgccaagctgggtatcacagagcctcagactcctggaacaggaactgtgggggtcaggtcagcaggggaggttagggagtgttccctttgtactgacttagcatttatcctgcttctaggggggaaggggggccagtgggggatgcacagcaaggcagtgatgtggcaggcagcctgcgggagctcctggttcctggtgtgaaaaagctgggaaggaagagggctgggtctggtaagtacagcaggcagttggctcctgagagtccaagccctgtctagagggtggagtgagatttcagagggagagctaaacggggtgggggctggggagtccaggcttctggctcctgctaatactcagtgtgctgggtcctcagaacctcagggtggccattttcagggtgagagctctgtcctttggcacttctgcagactccagtatccagaggaataaagatggtactcttcctcagttcccttagtgagaggacacctttctctgaagggcttgggcagttgtcctgaaccattgcctgaaggaaggacttgactccagggacatagaatgggctcagcataagtcccctgtagtagagaaaggtcccctctctggtctccttagagatcctgtttccttggctgaggaagctagggtggatctttgtgtaagtgggtgtggatgctcactggaaatcaaaaggccccttggtgttagaccttggggtgccatgggagagttgatcactgagtgcgcccttacatgggggccagctgagaatggggctgcctctagctcgagaccatgatgcagggagtgagtgggggagttcaggatactcttaactaaagcagaggtctgtccccccagggaggggaggtcagaagaccctagggagatgccaaaggctagggttggcaccatgttgcaggctgtgtcttcaaggagatgataatcagaggaatcgaacctgcaaaagtgggccagtcttagatacactatagaggaataatcttctgaaacattctgtgtctcataggacctgcctgaggacccagccccagtgccagcacatacactggggcagtgagtagatagtatactttgttacatgggctggggggacatggcctgtgccctggaggggacttgaagacatccaaaaagctagtgagagggctcctagatttatttgtctccaagggctatatatagccttcctaacatgaacccttgggtaatccagcatgggcgctcccatatgccctggtttgattagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtccgtcgccctcgctctgtgcccacccccgcctccctcaggcacagcaggcgtggagaggatgcgcaggaggtaggaggtgggggacccagaggggctttgacgtcagcctggcctttaagaggccgcctgcctggcaagggccgtggagacagaactcgggaccaccagcttgcact

SEQ ID NO: 34 - DNA, 1353 bp, Artificial sequence
  Myelin basic protein (MBP) promoter
caccgtggctttaacacttagagaaaatgcatcccctctaatcaataagtcatcgacagtgggtagatggaggaacggcagtgcgtagtaggatgcgtgcaagcatagtctcgtgcatgggtgcatagatcgctgggcaggtggacaaggtgggggtggataaagaagtgggtagatgattgatgttaggtaaatatcactgggtggacagatgggtggtaggtggatggatggttagaatagtcagaagagggatggattgataaggtgaacagatgataaatgggtgatagactggaagggttgtcaaaagaggataagggaagtgtgagctagccgtatttctaaggtcagtaatagagttgggagaagaggttaagttacatccatttaaacctcacacgaagctgagagggaatggacttgctgccgttggtgaggaaagcgttgcatttcccgtgtgcttggttgtgaagtgctcaggtcccacatgaagcagtcaggttactgcggcttacagaggagccagatccaaatgccccgagtaagcacgtccccgagccagaggcctccagcggaatccgggagagggattgctcagtgccctgcttccctggactgtaagctgcagaaagatgtgggaagtcctgttctccactgagaacactaaaagcaccttttgtcaaacgaccgcttcacatctggggcttgtgcactggtggccttttaaaccagagacaacccacaagatacctaacctgcggggctctctggtacagtgagcaactcaggaaatgctttggcttgattgctgtgggctctcaggccatcgccctctggagtggttcttttaatgagaacctgaagattggcccctgagccatgtataccaagcaagctcaatccaggttagctccctctggttggggcaagctaacgtgctccttgggccccgcgcgtaactgtgcgttttataggagacagctagttcaagaccccaggaagaaagcggctttgtccccctctaggcctcgtacaggcccacattcatatctcattgttgttgcaggggaggcagatgcgatccagaacaatgggacctcggctgaggacacggcggtgacagactccaagcacacagcagacccaaagaataactggcaaggcgcccacccagctgacccagggaaccgcccccacttgatccgcctcttttcccgagatgccccgggaagggaggacaacaccttcaaagacaggccctcagagtccgacgagcttcagaccatccaagaagatcccacagcagcttccgaagaattctgcagtcgacggtaccgcgggcccgggatc

SEQ ID NO: 35 - DNA, 128 bp, Artificial sequence
  Truncated AAV2 5′ ITR
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 36 - DNA, 128 bp, Artificial sequence
  Truncated AAV2 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 37 - DNA, 122 bp, Artificial sequence
  SV40 polyadenylation signal
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 38 - DNA, 449 bp, Artificial sequence
  Rabbit beta-globin polyadenylation signal
gatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 39 - DNA, 592 bp, Artificial sequence
  CMV promoter and CMV enhancer sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 40 - DNA, 7008 bp, Artificial sequence
  pAAV-CAG-hIns-dmiRT
agtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttccactgaggcccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctaggaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcggccgctaattctagatcgcgaacaaacaccattgtcacactccagtatacacaaacaccattgtcacactccagatatcacaaacaccattgtcacactccaaggcgaacaaacaccattgtcacactccaaggctattctagatcgcgaattacatacttctttacattccagtatacattacatacttctttacattccagatatcattacatacttctttacattccaaggcgaattacatacttctttacattccaaggctacctgaggcccgggggtacctcttaattaactggcctcatgggccttccgctcactgcccgctttccagtcgggaaacctgtcgtgccagtcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaa

SEQ ID NO: 41 - protein, 110 aa, Artificial sequence
  H. sapiens insulin with furin cleavage sites
MALWMRLLPLLALLALWGPDPAAAFVNQHLCGSHLVEALYLVCGERGFFYTPRTKREAEDLQVGQVELGGGPGAGSLQPLALEGSRQKRGIVEQCCTSICSLYQLENYCN

SEQ ID NO: 42 - protein, 110 aa, Artificial sequence
  H. sapiens insulin mutant His-B10-Asp with furin cleavage sites
MALWMRLLPLLALLALWGPDPAAAFVNQHLCGSDLVEALYLVCGERGFFYTPRTKREAEDLQVGQVELGGGPGAGSLQPLALEGSRQKRGIVEQCCTSICSLYQLENYCN

SEQ ID NO: 43 - protein, 110 aa, Rattus norvegicus
MALWIRFLPLLALLILWEPRPAQAFVKQHLCGSHLVEALYLVCGERGFFYTPMSRREVEDPQVAQLELGGGPGAGDLQTLALEVARQKRGIVDQCCTSICSLYQLENYCN

SEQ ID NO: 44 - protein, 110 aa, Pan troglodytes
MALWMRLLPLLVLLALWGPDPASAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 45 - DNA, 333 bp, Artificial sequence
  H. sapiens insulin with furin cleavage sites
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 46 - DNA, 333 bp, Artificial sequence
  H. sapiens insulin mutant His-B10-Asp with furin cleavage sites
atggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactag

SEQ ID NO: 47 - DNA, 333 bp, Rattus norvegicus
atggccctgtggatccgcttcctgcccctgctggccctgctcatcctctgggagccccgccctgcccaggcttttgtcaaacagcacctttgtggttctcacttggtggaagctctctacctggtgtgtggggagcgtggattcttctacacacccatgtcccgccgcgaagtggaggacccacaagtggcacaactggagctgggtggaggcccgggggcaggtgaccttcagaccttggcactggaggtggcccggcagaagcgcggcatcgtggatcagtgctgcaccagcatctgctctctctaccaactggagaactactgcaactag

SEQ ID NO: 48 - DNA, 333 bp, Pan troglodytes
atggccctgtggatgcgcctcctgcccctgctggtgctgctggccctctggggacctgacccagcctcggcctttgtgaaccaacacctgtgcggctcccacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggtatcgtggaacaatgctgtaccagcatctgctccctctaccagctggagaactactgcaactag

SEQ ID NO: 49 - DNA, 6740 bp, Artificial sequence
  pAAV-CAG-hInsAsp
agtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttccactgaggcccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagcgtcgaccttctgccatggccctgtggatgcgcctcctgcccctgctggcgctgctggccctctggggacctgacccagccgcagcctttgtgaaccaacacctgtgcggctcagatctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactagacgcagccgtcgacggtacccccgacgcggcctaactggcctcatgggccttccgctcactgcccgctttccagtcgggaaacctgtcgtgccagtcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaa

SEQ ID NO: 50 - DNA, 6740 bp, Artificial sequence
  pAAV-CAG-hInsWt
agtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttccactgaggcccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagcgtcgaggggtcgacatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcgcgacagaagcgtggcattgtggaacaatgctgtaccagcatctgctccctctaccagctggagaactactgcaactaggtcgacccctcgacggtacccccgacgcggcctaactggcctcatgggccttccgctcactgcccgctttccagtcgggaaacctgtcgtgccagtcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaa